(19) United States
(12) Patent Application Publication
(10) Pub. No.: US 2025/0281216 A1
(43) Pub. Date: Sep. 11, 2025

(54) DEVICES, SYSTEMS, AND METHODS FOR PULSED ELECTRIC FIELD TREATMENT OF TISSUE

(71) Applicant: Endogenex, Inc., Plymouth, MN (US)

(72) Inventors: Robert Anthony D'AGOSTINO, St. Louis Park, MN (US); Matthew HEIDNER, Maple Grove, MN (US); Zackary BECKER, Shoreview, MN (US); John POCRNICH, Roseville, MN (US); Dennis LARSON, Roseville, MN (US)

(21) Appl. No.: 19/066,736

(22) Filed: Feb. 28, 2025

Related U.S. Application Data
(60) Provisional application No. 63/563,149, filed on Mar. 8, 2024.

(51) Int. Cl. A61B 18/00

(52) U.S. Cl. CPC A61B 18/00; A61B 2018/00214; A61B 2018/00702; A61B 2018/00726; A61B 2018/00761

(57) ABSTRACT

Described here are devices, systems, and methods for applying pulsed or modulated electric fields to tissue. In some variations, a method of treating a target tissue may comprise advancing a pulsed electric field device and a visualization device to the target tissue of a patient. The pulsed electric field device may comprise an elongate body and an expandable member coupled to the elongate body. A suction catheter may be advanced from a lumen of the visualization device. Suction may be applied to the portion of the target tissue through the one or more fluid openings of the expandable member using the suction catheter. A pulsed waveform may be delivered to an electrode array of the pulsed electric field device to generate a pulsed or modulated electric field thereby treating the target tissue.

### RELATED APPLICATIONS

This application claims priority to provisional application No. 63/563,149, filed Mar. 8, 2024 and titled DEVICES, SYSTEMS, AND METHODS FOR PULSED ELECTRIC FIELD TREATMENT OF TISSUE, the entire content of which is incorporated herein by reference.

### TECHNICAL FIELD

Devices, systems, and methods herein relate to applying pulsed electric fields to tissue to treat a chronic disease, including but not limited to diabetes.

### BACKGROUND

Diabetes is a widespread condition, affecting millions worldwide. In the United States alone, over 20 million people are estimated to have the condition. Diabetes accounts for hundreds of billions of dollars annually in direct and indirect medical costs. Depending on the type (Type 1, Type 2, and the like), diabetes may be associated with one or more symptoms such as fatigue, blurred vision, and unexplained weight loss, and may further be associated with one or more complications such as hypoglycemia, hyperglycemia, ketoacidosis, neuropathy, and nephropathy.

The treatment of chronic diseases such as obesity and diabetes through duodenal resurfacing has been proposed. For example, removing the majority of the mucosal cells from the section of the large intestine nearest the stomach may allow a rejuvenated mucosal layer to be regenerated, thereby restoring healthy (non-diabetic) signaling. Conventional treatments that apply thermal energy to the duodenum risk excessively heating and thus damaging more layers of the duodenum (e.g., muscularis) than desired, and/or must compensate for this excessive thermal heating. Conversely, conventional solutions may generate incomplete and/or uneven treatment. As such, additional systems, devices, and methods for treatment of duodenal tissue may be desirable.

### SUMMARY

Described here are devices, systems, and methods for applying pulsed or modulated electric fields to tissue. These systems, devices, and methods may, for example, treat duodenal tissue of a patient to treat diabetes. In some variations, a method of treating a target tissue may comprise advancing a pulsed electric field device and a visualization device to the target tissue of a patient. The pulsed electric field device may comprise an elongate body and an expandable member coupled to the elongate body. The expandable member may comprise an electrode array and one or more fluid openings. The expandable member may be transitioned into an expanded configuration. A suction catheter may be advanced from a lumen of the visualization device. Suction may be applied to the portion of the target tissue through the one or more fluid openings of the expandable member using the suction catheter. A pulsed waveform or pulsed waveforms may be delivered to the electrode array to generate a pulsed or modulated electric fields thereby treating the target tissue.

In some variations, the visualization device may be positioned proximally of the expandable member while a) advancing the pulsed electric field device, b) transitioning the expandable member, c) applying suction, and d) delivering the pulsed waveform. In some variations, the expandable member may be visualized with the visualization device while the visualization device is positioned proximally of the expandable member.

In some variations, the expandable member in the expanded configuration defines an expandable member lumen. The suction catheter may be advanced from the lumen of the visualization device into the expandable member lumen. In some variations, the suction catheter may comprise a wire. In some variations, a proximal end of the wire comprises a loop. In some variations, the suction catheter may comprise a suction lumen and a diameter of the wire may be less than a diameter of the suction lumen. In some variations, a diameter of the loop may be larger than a diameter of the lumen of the visualization device. In some variations, a proximal end of the suction lumen may couple to a distal end of the wire.

In some variations, advancing the suction catheter may include aligning a distal end of the visualization device longitudinally with a proximal end of the expandable member. In some variations, a distal end of the suction catheter may comprise a plurality of apertures spaced around a circumference of the suction catheter. In some variations, the plurality of apertures may comprise opposing pairs of apertures.

In some variations, each aperture of the plurality of apertures may comprise a diameter of up to an inner diameter of the suction catheter. In some variations, the pulsed waveform may be delivered simultaneously with applying suction to the portion of the target tissue. In some variations, transitioning the expandable member into the expanded configuration dilates a portion of the target tissue. In some variations, the suctioned tissue may be received through the one of more fluid openings when applying the suction. In some variations, the target tissue may be duodenal tissue proximal and/or distal to an ampulla of Vater. In some variations, the target tissue may be tissue corresponding to a bulb of the duodenum. In some variations, the pulsed electric field device may be advanced together with the visualization device into the duodenum.

In some variations, the suction may be applied at least radially and longitudinally by the suction catheter. In some variations, treating the target tissue treats one or more of a metabolic disorder, pre-cancer, cancer, proinflammatory processes, immunological processes. In some variations, the metabolic disorder may comprise one or more of obesity, Non-alcoholic fatty liver disease (NAFLD), Nonalcoholic steatohepatitis (NASH), Type I diabetes, and Type II diabetes. In some variations, the target tissue may comprise one or more of a duodenum, a pylorus, an esophagus, a stomach, a small intestine, and a large intestine.

Also described herein is a suction catheter comprising a first elongate body comprising a proximal end, a distal end, and a lumen between the proximal and distal ends. The distal end of the first elongate body may comprise a plurality of apertures spaced around a circumference of the first elongate body. A second elongate body may be coupled to a proximal end of the first elongate body. A diameter of the second elongate body may be less than a diameter of the first elongate body.

In some variations, the second elongate body may comprise one or more of a wire, a cable, a coil, and a braid. In some variations, the plurality of apertures may comprise opposing pairs of apertures, wherein each aperture of the plurality of apertures may comprise a shape including one or more of, without limitation, a circle, ellipse, and polygon. In some variations, a diameter of each aperture of the plurality of apertures may be equal to or less than an inner diameter of the suction catheter.

In some variations, a proximal end of the second elongate body may comprise a loop. In some variations, a diameter of the second elongate body may be less than a diameter of the lumen. In some variations, a diameter of the loop may be larger than a diameter of the lumen. In some variations, the second elongate body may be coupled to an inner surface of the lumen at a proximal end of the first elongate body. In some variations, the suction catheter may be configured to apply suction at least radially and longitudinally through the plurality of apertures.

In some variations, a pulsed electric field device may comprise an elongate body and an expandable member coupled to the elongate body. The expandable member may comprise an electrode array and one or more fluid openings. The suction catheter may be configured to apply suction to tissue through the one or more fluid openings of the expandable member. In some variations, the expandable member may comprise an expandable member lumen in an expanded configuration. The first elongate body may be configured to be received within the expandable member lumen to assist in applying suction through the one or more fluid openings of the expandable member. In some variations, a proximal end of the second elongate body may be configured to restrict translation of the suction catheter through the visualization device.

Also described herein is a device comprising a first elongate body comprising a proximal portion, a distal portion, and a lumen therethrough. An expandable member may comprise an electrode array, an inner end, and an outer end coupled to the first elongate body. The expandable member may be disposed between the proximal portion and the distal portion. A tissue barrier may comprise a first portion and a second portion. The first portion may be coupled to a distal edge of the expandable member and to the distal portion of the elongate body, and the second portion may be coupled to a proximal edge of the expandable member and to the proximal portion of the elongate body.

In some variations, the tissue barrier may be rolled, or folded, about the first elongate body. In some variations, a second elongate body may be at least partially positioned within the lumen of the first elongate body. The expandable member may be rolled about the second elongate body, and the inner end is coupled to the second elongate body.

In some variations, the proximal portion may comprise a proximal dilator and the distal portion comprise a distal dilator. In some variations, the second elongate body may be configured to rotate relative to the first elongate body to transition the tissue barrier between a rolled configuration and an unrolled configuration, or a folded configuration and an unfolded configuration.

In some variations, the tissue barrier may be configured to transition between a rolled configuration and an unrolled configuration, or a folded and an unfolded configuration in some variations. In some variations, the first and second portions of the tissue barrier in the unrolled, or unfolded, configuration may form a pair of right triangles. In some variations, each triangle of the pair of right triangles may comprise an acute angle of between about 20 degrees and about 90, though acute angles less than 20 degrees may also be provided and are within the scope of the present disclosure. In some variations, a shape of the first portion and the second portion may be the same, while in other embodiments, the first and second portions may comprise different shapes. In some variations, the tissue barrier may comprise a plurality of turns about the first elongate body.

In some variations, the tissue barrier may be rolled or folded about a longitudinal axis of the first elongate body. In some variations, the tissue barrier may comprise a durometer between about 10 Shore A and about 100 Shore A, though other durometers may occur to the skilled artisan and are within the scope of the present disclosure. In some variations, a distance between the first portion and the second portion may be between about 3 mm and about 20 mm, though other distances may be provided, all of which are within the scope of the present disclosure. In some variations, the tissue barrier may comprise a thickness of between about 0.125 mm and about 0.8 mm, though other thicknesses may be provided, each of which is within the scope of the present disclosure. In some variations, the tissue barrier may couple to each of the proximal edge and the distal edge for a length of between about 25 mm and about 130 mm, though other coupling lengths may be provided and are within the scope of the present disclosure.

Also described herein is a method of treating a target tissue comprising advancing a pulsed electric field device to the target tissue of a patient. The pulsed electric field device may comprise an elongate body, a tissue barrier, and an expandable member coupled to the elongate body. The expandable member may comprise an electrode array and the elongate body comprises a proximal portion and a distal portion. The expandable member may transition into an expanded configuration to contact a portion of the target tissue and to expand the tissue barrier. A pulsed waveform may be delivered to the electrode array to generate a pulsed or modulated electric field thereby treating the target tissue.

In some variations, the tissue barrier may comprise a first portion and a second portion, the first portion coupled to a proximal edge of the expandable member and to the proximal portion of the elongate body, and the second portion coupled to a distal edge of the expandable member and to the distal portion of the elongate body.

In some variations, transitioning the expandable member may comprise unrolling one or more turns of the expandable member. In some variations, transitioning the expandable member may comprise unrolling one or more turns of the tissue barrier about the first elongate body. In some variations, transitioning the expandable member may comprise rotating the second elongate body relative to the first elongate body. In some variations, the tissue barrier may be configured to transition between a rolled configuration and an unrolled configuration.

In some variations, the tissue barrier in the unrolled configuration may comprise a pair of right triangles. In other variations, the tissue barrier in the unrolled configuration may comprise a pair of triangles, wherein one or both are selected from the group consisting of a right triangle, an equilateral triangle, an isosceles triangle, a scalene triangle, an acute triangle and an obtuse triangle.

Also described herein is a device comprising a first elongate body comprising a lumen, an expandable member comprising an inner end, an outer end coupled to the first elongate body, and an electrode array. The expandable member may be disposed between a proximal portion and a distal portion of the first elongate body. A cover may be coupled between the outer end of the expandable member and the first elongate body. The cover may be configured to overlap a portion of the expandable member.

In some variations, the device may include a second elongate body at least partially positioned within the lumen. The expandable member may be rolled about the second elongate body, and the inner end is coupled to the second elongate body.

In some variations, the cover may overlap the portion of the expandable member as the expandable member rolls about the first elongate body. In some variations, the cover may comprise a concave shape having a radius of curvature between about 6 mm and about 16 mm, though other radii may be provided all of which are within the scope of the present disclosure. In some variations, the cover may comprise a width of between about 23 mm and about 38 mm, though other widths may be provided, all of which are within the scope of the present disclosure. In some variations, a length of the cover may be greater than or equal to a length of the expandable member. In some variations, the cover may comprise one or more atraumatic edges.

In some variations, the cover may be configured to reduce contact between tissue and the second elongate body. In some variations, the cover may comprise a slit configured to slidably receive the expandable member therethrough. In some variations, the cover may comprise a first portion facing an outer side of the expandable member and a second portion facing an inner side of the expandable member. In some variations, the second portion may be coupled to the first elongate body. In some variations, the slit may be parallel to a longitudinal axis of the first elongate body. In some variations, rolling and unrolling the expandable member may translate the expandable member through the slit.

Also described herein is a system for treating tissue comprising a pulsed electric field device configured and comprising an elongate body and an expandable member coupled to the elongate body. The expandable member may comprise an electrode array having a plurality of sections. A signal, or pulse, generator may be coupled to the electrode array. The signal generator may be configured to deliver a pulsed electric field waveform to two or more non-proximate sections of the plurality of sections in a predetermined sequence, with a predetermined pulse width, and a predetermined magnitude. The predetermined sequence may comprise an inter-section delay between delivery of a first pulsed electric field waveform to a first section of the plurality of sections and a second pulsed electric field waveform to a second section of the plurality of sections.

In some variations, the inter-section delay is between about 10 ms and about 4000 ms, though shorter or longer delays may be provided, all of which are within the scope of the present disclosure. In some variations, the first and second sections are non-proximate, or non-adjacent, sections. In some variations, the predetermined sequence may further comprise an intra-section delay between delivery of the first pulsed electric field waveform to the first section and delivery of a second pulsed electric field waveform to the first section. In some variations, the intra-section delay may be between about 1 seconds and about 10 seconds, though shorter or longer delays may be provided, each of which is within the scope of the present disclosure.

In some variations, the first and second pulsed electric field waveforms may comprise a series of between about 10 bipolar pulses and about 500 bipolar pulses, though other pulse numbers in a series may be provided, each of which is within the scope of the present invention. Further, a plurality of series of pulses may be provided in some variations. In some of these variations, the number of pulses in each series may be equal, while in other variations, the number of pulses in one or more of the series may be different than the number of pulses in at least one other series. For example and without limitation, in some variations, the first and second pulsed electric field waveforms may comprise the same number of bipolar pulses. In some variations, the first and second pulsed electric field waveforms may comprise a different number of bipolar pulses. In some variations, each of the bipolar pulses may comprise a pulse width between about 1 μs and about 10 μs, though larger or smaller pulse widths may be provided and are within the scope of the present disclosure.

In some variations, the electrode array may be configured to deliver between about 0.05 J per bipolar pulse and about 0.5 J per bipolar pulse. In some variations, the electrode array may be configured to deliver an instantaneous power between about 26,000 W per bipolar pulse and about 70,000 W per bipolar pulse.

In some variations, the signal generator may be configured to repeat the predetermined sequence between about 5 and about 15 times, though a larger or small number of cycles of the predetermined sequence may be provided, each of which is within the scope of the present disclosure. In some variations, the signal generator may be configured to activate the plurality of sections for a cumulative activation time between about 0.1 ms and about 10 ms over a treatment time between about 30 seconds and about 35 seconds, though additional cumulative activation times and total treatment times may be provided, each of which is within the scope of the present disclosure. In some variations, the predetermined sequence may comprise a duty cycle of between about 0.001% and about 0.05%, though other duty cycles may be provided, each of which is within the scope of the present disclosure.

In some variations, the plurality of electrode sections may comprise up to about ten electrode sections. In other variations, more than 10 electrode sections may be provided. In some variations, the electrode array may comprise a surface area between about 4 square centimeters and about 42 square centimeters, though greater or smaller surface areas may be provided each of which is within the scope of the present disclosure. In some variations, each electrode section of the plurality of electrode sections may comprise a plurality of electrodes. In some variations, each electrode section of the plurality of electrode sections may comprise between about 8 electrodes and about 18 electrodes, though the number of electrodes in each electrode section may be less than 8 electrodes or greater than 18 electrodes. In some variations, the numbers of electrodes in each electrode section are equal. In other variations, the numbers of electrodes in each electrode section are not equal.

Also described herein is a method of treating tissue comprising advancing a pulsed electric field device to a target tissue of a patient. The pulsed electric field device may comprise an elongate body and an expandable member coupled to the elongate body. The expandable member may comprise an electrode array having at least a first section and a second section coupled to a signal generator. The first section may be non-proximate to the second section. Tissue may be treated by providing a pulsed electric field waveform from the signal generator to each of the first and second sections in a predetermined sequence to activate the first section followed by the second section after an inter-section delay. Activation of each of the first and second sections may generate a therapeutic electric field.

In some variations, the delay may be between about 10 ms and about 4000 ms, though other delay times may be provided, each of which is within the scope of the present disclosure. In some variations, the first section may be re-activated after an intra-section delay relative to a previous activation of the first section. In some variations, the intra-section delay may be between about 1 seconds and about 10 seconds, though other intra-section delay times may be provided each of which is within the scope of the present invention. In some variations, the pulsed electric field waveform may comprise a series of between about 10 bipolar pulses and about 500 bipolar pulses. Other variations may comprise one or more series of pulses that are less than 10 and/or greater than 500 pulses. In some variations, each of the bipolar pulses may comprise a pulse width between about 1 μs and about 10 μs, though other pulse widths are possible, each of which is within the scope of the present invention. In some variations, activating each of the first and second sections may deliver between about 0.05 J per bipolar pulse and about 0.5 J per bipolar pulse. Other variations may deliver less than 0.05 J per pulse and/or greater than about 05 J per pulse.

In some variations, activating each of the first and second sections may deliver an instantaneous power between about 26,000 W per bipolar pulse and about 70,000 W per bipolar pulse, though other power magnitudes are possible and within the scope of the present disclosure. In some variations, the pulsed electric field waveform delivered to the first electrode section may comprise a different number of bipolar pulses than the pulsed electric field waveform delivered to the second electrode section. In some variations, the pulsed electric field waveform delivered to the first electrode section may comprise an equivalent number of bipolar pulses as the pulsed electric field waveform delivered to the second section. In some variations, the first and second electrode sections may be non-proximate or non-adjacent to each other.

In some variations, the electrode array may further comprise one or more of a third electrode section, a fourth electrode section, and a fifth electrode section. In some variations, more than five electrode sections may be provided. In some variations, the predetermined sequence of individual series of voltage pulses may further comprise activating the one or more of the third section, fourth section, and fifth section with an inter-section delay between activation of successive electrode sections in the sequence. In some variations, the first and second electrode sections may be activated for a cumulative activation time between about 0.1 ms and about 10 ms over a treatment time between about 30 and about 35 seconds. In some variations, the predetermined sequence of pulses may comprise a duty cycle between about 0.001% and about 0.05%. In some variations, the predetermined sequence may be repeated between about 5 and about 15 times. Variations of each of the foregoing parameters may be outside of the provided ranges while still within the scope of the present disclosure.

Also described herein is a system of treating tissue comprising a pulsed electric field device comprising an elongate body and an expandable member coupled to the elongate body. The expandable member may comprise an electrode array having a plurality of sections. A signal generator may be coupled to the electrode array. The signal generator may be configured to deliver a series of bipolar pulses to two or more non-proximate sections of the plurality of sections in a predetermined sequence for a cumulative activation time of between about 1 ms and about 2 ms over a treatment period between about 30 seconds and about 35 seconds. Each bipolar pulse may comprise a pulse width between about 2.5 μs and about 3 μs. The electrode array may be configured to deliver between about 0.1 J per bipolar pulse and about 0.2 J per bipolar pulse and an instantaneous power between about 38,800 W per bipolar pulse and about 41,250 W per bipolar pulse. Variations of each of the foregoing parameters may be outside of the provided ranges while still within the scope of the present disclosure.

In some variations, each of the bipolar pulses may provide a waveform comprise a positively-charged portion and a negatively-charged portion each having a pulse width between about 1.3 μs and about 1.5 μs. In some variations, each of the bipolar pulses may comprise a time interval between the positively-charged and negatively-charged portions. In some variations, the time interval may be between about 0.05 μs and about 0.1 μs. Variations of each of the foregoing parameters may be outside of the provided ranges while still within the scope of the present disclosure.

In some variations, the plurality of electrode sections may comprise about ten electrode sections. The electrode array may comprise a surface area between about 4 square centimeters and about 5 square centimeters. In some variations, the signal generator may be configured to deliver a pulse waveform to each of the plurality of sections in a predetermined sequence comprising an inter-section delay between delivering the pulse waveform to successive sections. In some variations, the inter-section delay may be between about 500 ms and about 1000 ms. In some variations, the pulse waveform may comprise a series of between 40 and 60 bipolar pulses. In some variations, the predetermined sequence may be repeated between about 5 and about 15 times according to a predetermined sequence. Variations of each of the foregoing parameters may be outside of the provided ranges while still within the scope of the present disclosure.

### BRIEF DESCRIPTION OF THE DRAWINGS

FIG. 1A is a cross-sectional representation of a gastrointestinal tract showing various anatomical structures.

FIG. 1B is a cross-sectional representation of a duodenum.

FIG. 2A is a cross-sectional schematic view of a portion of the small intestine.

FIG. 2B is a cross-sectional schematic view of a portion of the small intestine.

FIG. 2C is a cross-sectional schematic view of a portion of the small intestine.

FIG. 3A is a cross-sectional image of a duodenum.

FIG. 3B is a detailed cross-sectional image of duodenal tissue.

FIG. 3C is a detailed cross-sectional image of duodenal tissue.

FIG. 3D is a detailed cross-sectional image of duodenal tissue.

FIG. 3E is a detailed cross-sectional image of duodenal tissue.

FIG. 3F is a detailed cross-sectional image of duodenal tissue.

FIG. 4 is a block diagram of an illustrative variation of a pulsed electric field system.

FIG. 5A is a plan view of an illustrative variation of a pulsed electric field device in a rolled or unexpanded configuration.

FIG. 5B is a plan view of an illustrative variation of a pulsed electric field device in an unrolled or expanded configuration.

FIG. 5C is a bottom view of an illustrative variation of a pulsed electric field device in a rolled or unexpanded configuration.

FIG. 5D is a bottom view of an illustrative variation of a pulsed electric field device in an unrolled or expanded configuration.

FIG. 5E is a left side view of an illustrative variation of a pulsed electric field device in a rolled or unexpanded configuration.

FIG. 5F is a left side view of an illustrative variation of a pulsed electric field device in an unrolled or expanded configuration.

FIG. 5G is a right side view of an illustrative variation of a pulsed electric field device in a rolled or unexpanded configuration.

FIG. 5H is a right side view of an illustrative variation of a pulsed electric field device in a rolled or unexpanded configuration.

FIG. 5I is a right side view of an illustrative variation of a pulsed electric field device in an unrolled or expanded configuration.

FIG. 5J is a right side view of an illustrative variation of a pulsed electric field device in an unrolled or expanded configuration.

FIG. 5K is a right side view of an illustrative variation of a pulsed electric field device in an unrolled or expanded configuration.

FIG. 5L is a perspective top view of an illustrative variation of a pulsed electric field device in a rolled or unexpanded configuration.

FIG. 5M is a bottom view of an illustrative variation of a pulsed electric field device in a rolled or unexpanded configuration.

FIG. 5N is a perspective view of an illustrative variation of a pulsed electric field device in an expanded configuration.

FIG. 5O is a perspective view of an illustrative variation of a pulsed electric field device in an expanded configuration.

FIG. 5P is a perspective view of an illustrative variation of a pulsed electric field device in an expanded configuration.

FIG. 5Q is a cross-sectional view of an illustrative variation of a pulsed electric field device in a rolled or unexpanded configuration.

FIG. 5R is a cross-sectional view of an illustrative variation of a pulsed electric field device in an unrolled or expanded configuration.

FIG. 6A is a schematic view of illustrative variations of a tissue barrier of a pulsed electric field device.

FIG. 6B is a schematic view of illustrative variations of a tissue barrier of a pulsed electric field device.

FIG. 6C is a schematic view of illustrative variations of a tissue barrier of a pulsed electric field device.

FIG. 6D is a schematic view of illustrative variations of a tissue barrier of a pulsed electric field device.

FIG. 6E is a schematic view of illustrative variations of a tissue barrier of a pulsed electric field device.

FIG. 6F is a schematic view of illustrative variations of a tissue barrier of a pulsed electric field device.

FIG. 6G is a schematic view of illustrative variations of a tissue barrier of a pulsed electric field device.

FIG. 7A is a schematic view of illustrative variations of a cover of a pulsed electric field device.

FIG. 7B is a schematic view of illustrative variations of a cover of a pulsed electric field device.

FIG. 7C is a schematic view of illustrative variations of a cover of a pulsed electric field device.

FIG. 7D is a schematic view of illustrative variations of a cover of a pulsed electric field device.

FIG. 8 is a perspective view of an illustrative variation of an expandable member comprising an electrode array in an unrolled configuration.

FIG. 9A is a perspective view of an illustrative variation of an expandable member in a rolled or unexpanded configuration.

FIG. 9B is a perspective view of an illustrative variation of an expandable member in an unrolled or expanded configuration.

FIG. 10A is a perspective view of an illustrative variation of a distal portion of a pulsed electric field device.

FIG. 10B is a cross-sectional side view of the pulsed electric field device shown in FIG. 10A.

FIG. 10C is a detailed cutaway perspective view of the pulsed electric field device shown in FIG. 10A.

FIG. 10D is a detailed side view of an illustrative variation of a dilator of a pulsed electric field device.

FIG. 11A is a perspective view of an illustrative variation of an expandable member.

FIG. 11B is a plan view of the expandable member shown in FIG. 11A in an unrolled configuration.

FIG. 11C is a cross-sectional view of an illustrative variation of an expandable member in a rolled configuration and gear.

FIG. 12A is a side view of an illustrative variation of a suction catheter.

FIG. 12B is an image of an illustrative variation of a suction catheter and visualization device.

FIG. 13 is a flowchart describing an illustrative variation of a method of treating tissue.

FIG. 14A is a schematic view of an illustrative variation of a method of treating tissue using a pulsed electric field device, a suction catheter, and a visualization device.

FIG. 14B is a schematic view of an illustrative variation of a method of treating tissue using a pulsed electric field device, a suction catheter, and a visualization device.

FIG. 14C is a schematic view of an illustrative variation of a method of treating tissue using a pulsed electric field device, a suction catheter, and a visualization device.

FIG. 14D is a schematic view of an illustrative variation of a method of treating tissue using a pulsed electric field device, a suction catheter, and a visualization device.

FIG. 14E is a schematic view of an illustrative variation of a method of treating tissue using a pulsed electric field device, a suction catheter, and a visualization device.

FIG. 14F is a schematic view of an illustrative variation of a method of treating tissue using a pulsed electric field device, a suction catheter, and a visualization device.

FIG. 14G is a schematic view of an illustrative variation of a method of treating tissue using a pulsed electric field device, a suction catheter, and a visualization device.

FIG. 14H is a schematic view of an illustrative variation of a method of treating tissue using a pulsed electric field device, a suction catheter, and a visualization device.

FIG. 14I is a schematic view of an illustrative variation of a method of treating tissue using a pulsed electric field device, a suction catheter, and a visualization device.

FIG. 14J is a schematic view of an illustrative variation of a method of treating tissue using a pulsed electric field device, a suction catheter, and a visualization device.

FIG. 14K is a schematic view of an illustrative variation of a method of treating tissue using a pulsed electric field device, a suction catheter, and a visualization device.

FIG. 14L is a schematic view of an illustrative variation of a method of treating tissue using a pulsed electric field device, a suction catheter, and a visualization device.

FIG. 15A is an image of an illustrative variation of a method of treating tissue using a pulsed electric field device, a suction catheter, and a visualization device.

FIG. 15B is an image of an illustrative variation of a method of treating tissue using a pulsed electric field device, a suction catheter, and a visualization device

FIG. 15C is an image of an illustrative variation of a method of treating tissue using a pulsed electric field device, a suction catheter, and a visualization device

FIG. 15D is an image of an illustrative variation of a method of treating tissue using a pulsed electric field device, a suction catheter, and a visualization device

FIG. 15E is an image of an illustrative variation of a method of treating tissue using a pulsed electric field device, a suction catheter, and a visualization device

FIG. 15F is an image of an illustrative variation of a method of treating tissue using a pulsed electric field device, a suction catheter, and a visualization device

FIG. 15G is an image of an illustrative variation of a method of treating tissue using a pulsed electric field device, a suction catheter, and a visualization device

FIG. 15H is an image of an illustrative variation of a method of treating tissue using a pulsed electric field device, a suction catheter, and a visualization device

FIG. 15I is an image of an illustrative variation of a method of treating tissue using a pulsed electric field device, a suction catheter, and a visualization device

FIG. 15J is an image of an illustrative variation of a method of treating tissue using a pulsed electric field device, a suction catheter, and a visualization device

FIG. 15K and is an image of an illustrative variation of an expandable member of a pulse electric field device.

FIG. 15L is an image of an illustrative variation of an expandable member of a pulse electric field device.

FIG. 16 is a flowchart describing an illustrative variation of a method of treating tissue.

FIG. 17A is a schematic diagram of an illustrative variation of a pulse waveform for treating tissue.

FIG. 17B is a schematic diagram of an illustrative variation of a pulse waveform for treating tissue.

FIG. 18A is a schematic diagram of an illustrative variation of a method of treating tissue.

FIG. 18B is a schematic diagram of an illustrative variation of a method of treating tissue.

FIG. 18C is a schematic diagram of an illustrative variation of a method of treating tissue.

FIG. 19A is a schematic diagram of an illustrative variation of a method of treating tissue.

FIG. 19B is a schematic diagram of an illustrative variation of a method of treating tissue.

FIG. 19C is a schematic diagram of an illustrative variation of a method of treating tissue.

FIG. 19D is a schematic diagram of an illustrative variation of a method of treating tissue.

FIG. 19E is a schematic diagram of an illustrative variation of a method of treating tissue.

FIG. 19F is a schematic diagram of an illustrative variation of a method of treating tissue.

FIG. 20 is a block diagram of an illustrative variation of a signal generator.

FIG. 21A is a plot of dose-based changes in HbA1c in response to a duodenal mucosal resurfacing procedure for insulin naïve Type 2 diabetes patients.

FIG. 21B is a plot of time-in-range-based changes in response to a duodenal mucosal resurfacing procedure for insulin naïve Type 2 diabetes patients.

FIG. 21C is a plot of weight-based changes in response to a duodenal mucosal resurfacing procedure for insulin naïve Type 2 diabetes patients.

### DETAILED DESCRIPTION

Described herein are devices, systems, and methods for treating tissue to address a chronic disease. For example, a pulsed electric field (PEF) system may be configured to generate a therapeutic pulsed electric field having predetermined bipolar, high current, short duration, electric pulses and applied to any body cavity or lumen (e.g., organ, vasculature, vessel) of a patient. The PEF treatment described herein may increase cell permeability and induce a targeted, non-thermal cellular necrosis while preserving Extracellular Matrix (ECM) tissue scaffold, promoting rapid epithelial layer replacement that reestablishes a neuroendocrine cell population with minimal inflammation. In this manner, a depth of penetration may be controlled and smooth muscle cells may be preserved, thereby leaving surrounding tissue undamaged.

In some variations, devices, systems, and methods may include those for treating diabetes by treating tissue within the gastrointestinal tract (e.g., duodenal tissue) of a patient. In some variations, treatment of the duodenum may comprise treating at least about 30% of the mucosal lining of the duodenum with minimal trauma, damage or scarring to the submucosa, vasculature, and muscles. For example, a mucosa layer of the duodenum may be treated using a pulsed electric field (PEF) system configured to generate a therapeutic pulsed electric field. The application of a pulsed electric field to duodenal tissue may affect individual parts or mechanisms within a cell (e.g., depth of tissue treated), that can be specifically targeted based on electrode geometry and the frequency, intensity including the voltage magnitude and duration or pulse width of the pulses. In some variations, an expandable member to which the electrode array is attached comprises an outward radial compression force that also enables reasonable determination of the expanded location of the electrodes relative to the submucosa and/or the muscularis layers.

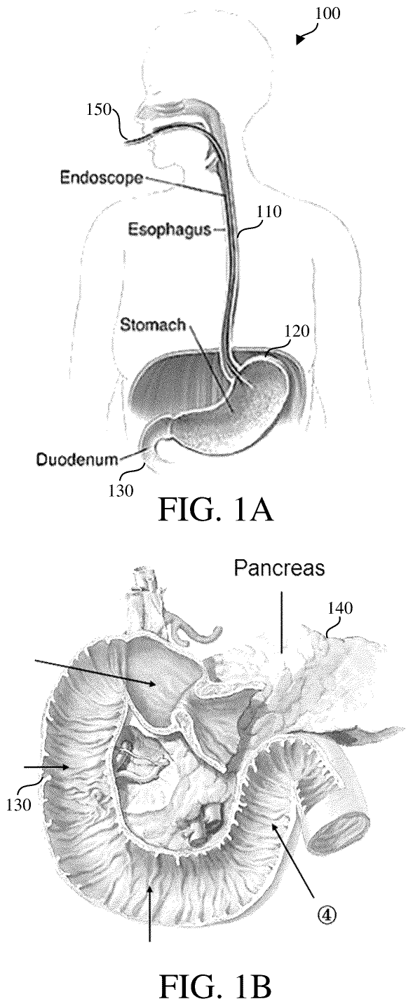

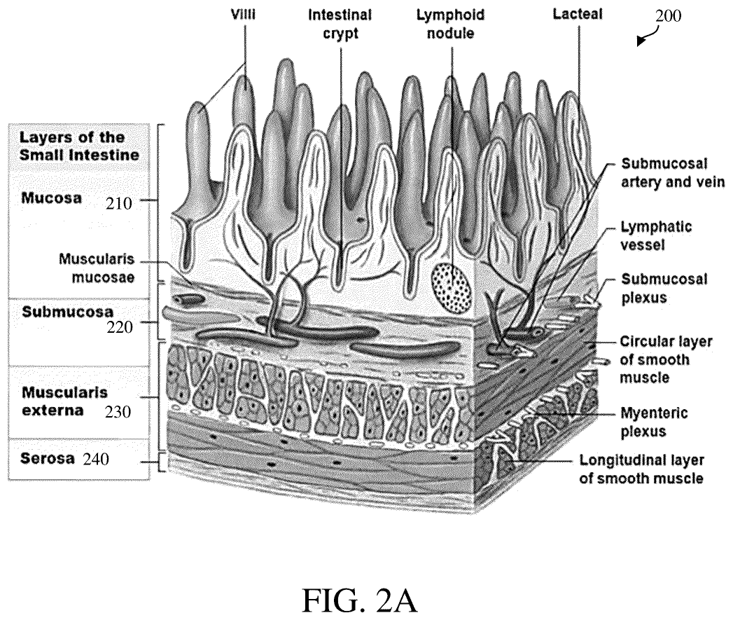

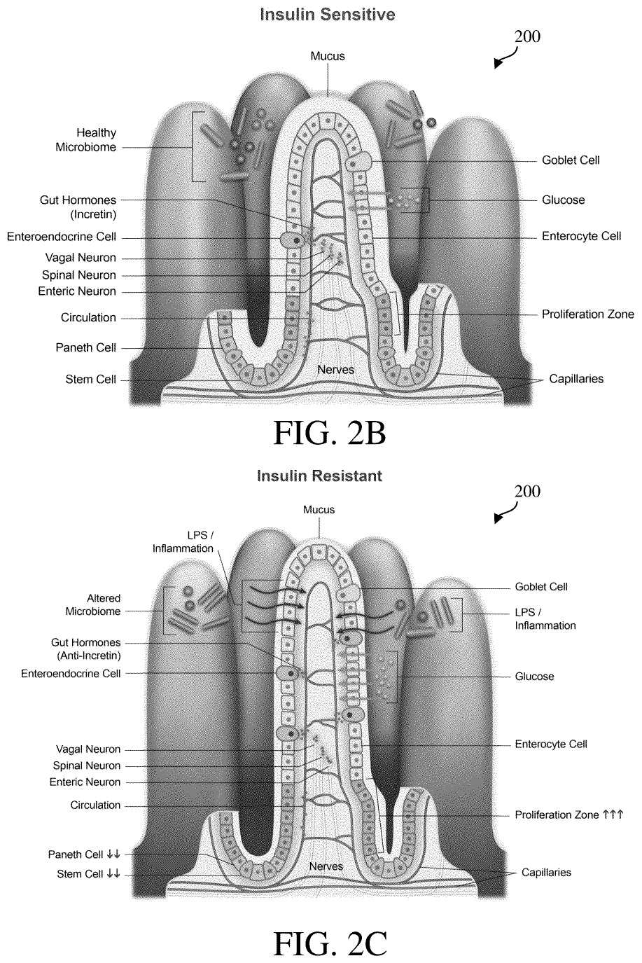

It may be helpful to briefly identify and describe the relevant small intestine anatomy. FIG. 1A is a cross-sectional view of the gastrointestinal tract of a patient (100). Shown there is a visualization device (150) (e.g., endoscope) advanced into the stomach (120) through the esophagus (110). The stomach (120) is connected to the duodenum (130). FIG. 1B is a detailed cross-sectional view of the duodenum (130), which surrounds the head of the pancreas (140). The duodenum is a “C” shaped hollow jointed tube structure that is typically between about 20 cm and about 35 cm in length and between about 20 mm and about 45 mm in diameter. FIGS. 2A-2C are cross-sectional schematic views of the layers of the small intestine (200) including the mucosa (210), submucosa (220), muscularis externa (230), and serosa (240). Treatment of the duodenum may comprise resurfacing the mucosa (210) as described herein. Access to the gastrointestinal tract (e.g., duodenum, stomach, large intestine) may be performed by advancing the systems and devices described herein through one or more of the esophagus, stomach, pylorus, lower esophageal junction, crackle pharyngeal junction, and several acute small radius bends throughout the length of the digestive tract.

It may further be helpful to briefly discuss electroporation and the role of ohmic heating. Electroporation is the application of an electric field to living cells to cause ions of opposite charge to accumulate on opposite sides of cell membranes. Generally, electroporation requires a potential difference across the cell membrane on the order of about 0.5 to about 1 volt and for a cumulative duration on the order of about 1 to about 2 milliseconds. Electroporation necessarily generates ohmic heating but there is considerable confusion in the literature about this, including a significant number of references that incorrectly assert the existence of non-thermal electroporation. For example, an external uniform electric field of magnitude E applied to an intracellular fluid with ionic conductivity σ_{ic} will generate a current density Eσ_{ic} and dissipate a thermal power density E²σ_{ic}. If the medium has a heat capacity C, and density p, the resulting rate of temperature rise is given by equation (1):

$$\frac{dT}{dt} = \frac{{E}^{2} {σ}_{ic}}{{C}_{p} ρ} \qquad equation ( 1 )$$

For example, a 1 KV/cm electric field acting on tissue with a conductivity of about 0.3 S/m, a heat capacity of about 3.7 joule/(gm° C.), and a density of about 1 gm/cc will heat the tissue at a rate of about 800° C./second. Note that, without current passing through the tissue, there is no electric field in the tissue since the tissue is an ionic conductor. The initial time after an external field is abruptly applied to the membrane to accumulate charge may be on the order of about 30 nanoseconds, which suggests that, during an initial membrane-charging phase, the average temperature rise may be in the tens of microdegrees. When an external electric field is applied, and ionic currents have charged the membrane surfaces to collapse the field into the lipid bilayers, leakage current may still flow, though the heating may be confined to the membranes for sub-microsecond timescales. For example, using a lipid layer conductivity of σₗᵢ=0.002 S/m, a 1-volt potential across an 8 nm layer may locally heat at an instantaneous rate of about 8° C./microsecond. This heating rate drops with time from the application of the external electric field, as the heat may diffuse further from the membrane.

If the ionic currents are confined to pores in the cell membranes, current crowding will cause the heating rate in the pores to be correspondingly higher. Since the pore area might be 1% or less of the membrane area, the current density in the pores may be one hundred times higher than in the bulk tissue. This gives a ten thousand times increase in heating rate, leading to local heating rates on the order of 10° C./microsecond.

Local temperature rise is a contributing mechanism to the transition from electroporation to irreversible electroporation. Thermal diffusion lowers the local temperature excursions. For example, assuming a tissue thermal diffusivity κ of 0.13 mm²/s, the thermal diffusion length at 10 μsec is \sqrt{(10 μs)(0.13 mm²/s)} or 1.1 micron, which is much larger than a typical pore. At 1 millisecond, the thermal diffusion length is on the order of the cell size, so the localized heating effects may be ignored.

The bulk tissue remains a good ionic conductor during the electroporation treatment, heating at a rate on an order of magnitude of about 800° C./s while the external field is being applied. If the external field is removed, the cell membranes may discharge on the order of about 30 nanoseconds, obliging the continued application of external voltage and current to induce pore formation and growth. As the maximum tolerable temperature rise of the bulk tissue may be on the order of about 13° C., the maximum duration that the external field may be applied, even in a bipolar configuration, may be within an order of magnitude of about 10 milliseconds. As this heat is generated to a treatment depth in the tissue of about several millimeters, the required time to cool the tissue by conduction may be about 70 seconds (e.g., (3 mm²)/(0.13 mm²/sec)). Blood convection likely dominates the observed cooling times that are on the order of about 10 seconds. Electroporation may also increase with the temperature of the bulk tissue due to the phase transition of the lipid cell membrane, which for some cells on the duodenum is 41° C. The phase transition temperature may be the temperature required to induce a change in the lipid physical state from the ordered gel phase to the liquid crystalline phase.

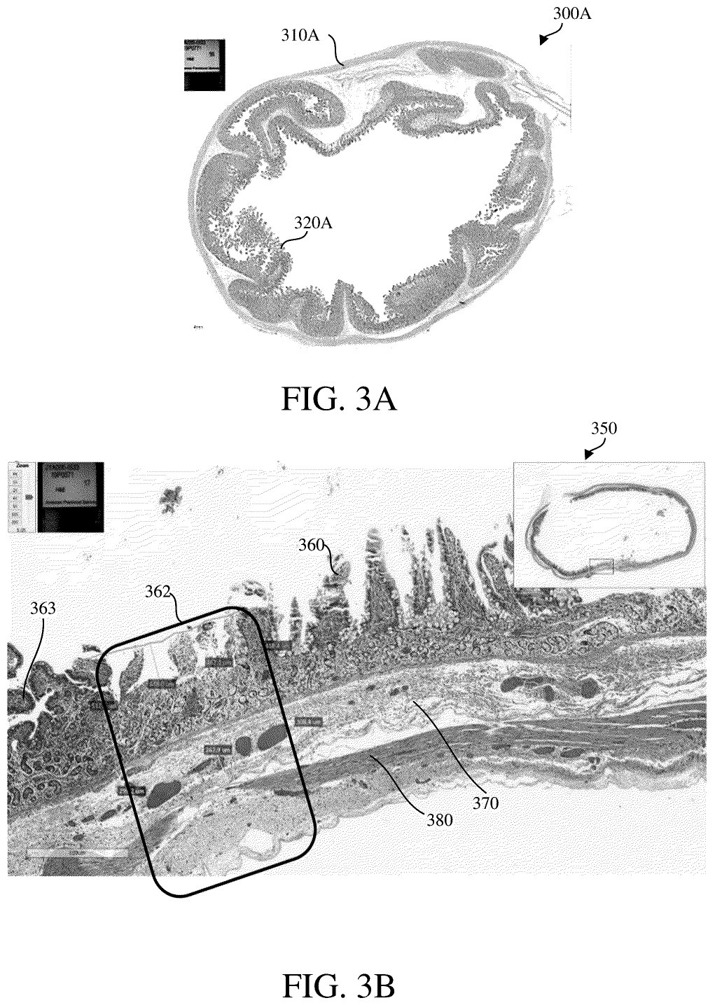

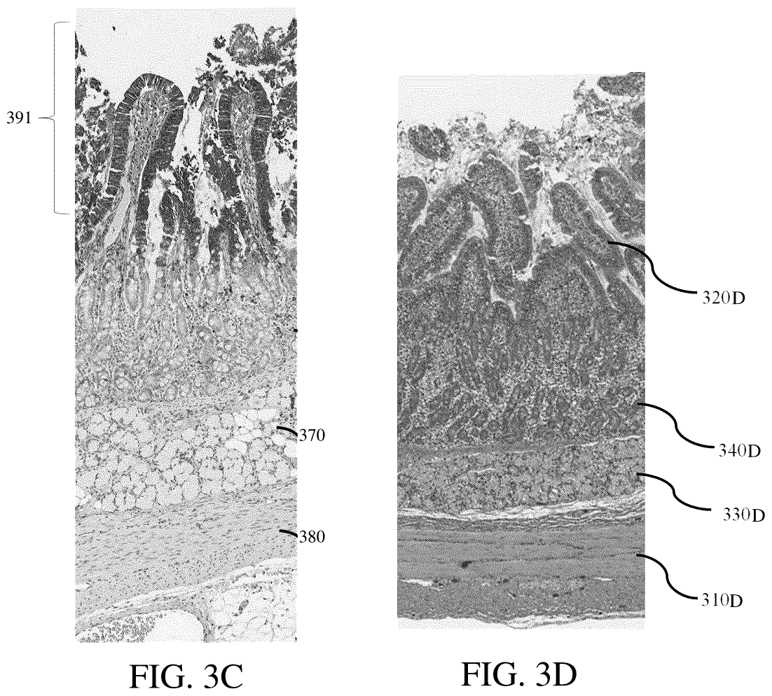

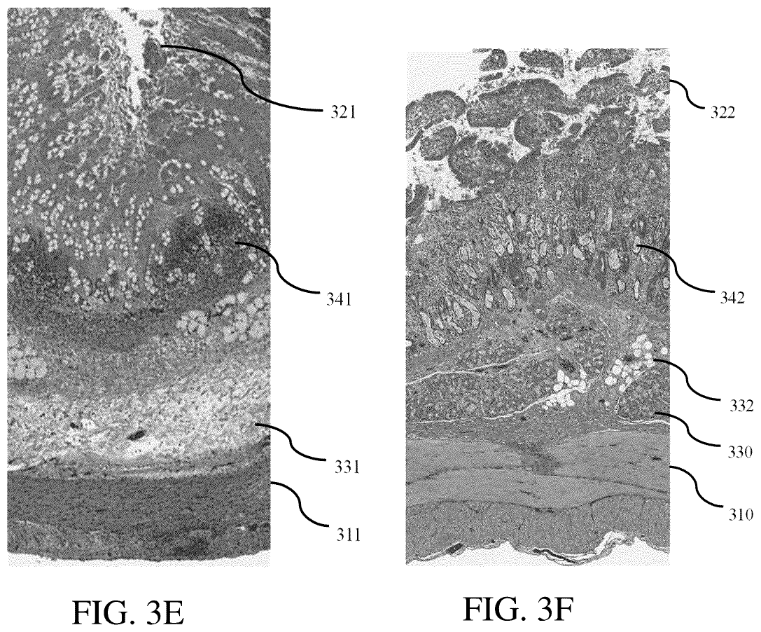

Electroporation parameters may be varied to produce different effects on tissue. FIG. 3A is a cross-sectional image of an untreated duodenum (300A) including a muscular layer (310A) and villi (320A). FIG. 3D is an image of an illustrative variation of duodenal tissue in its native untreated state including a muscularis layer (310D), submucosa (330D), villus crypts (340D) and villi (320D). As described in more detail herein, FIG. 3E depicts duodenal tissue that has undergone majority thermal heat treatment and FIG. 3F depicts duodenal tissue that has undergone majority pulsed or modulated electric field treatment. The treatments described herein (e.g., FIG. 3F), which primarily treat the mucosa layer with preserved tissue architecture appearing similar to the native tissue, reduces trauma to tissue relative to the thermal treatment shown in FIG. 3E.

The application of a pulsed electric field to tissue results in non-thermal tissue changes. For example, FIG. 3D is an image of normal untreated (e.g., native tissue) porcine duodenal mucosa. FIG. 3F is an image of the initial mucosal histologic appearance with evolving epithelial loss and lamina propria structural/architectural preservation. For example, FIG. 3F depicts the histologic evolution with complete native epithelial loss and early crypt regeneration within the preserved lamina propria. The glandular layer across FIGS. 3A-3D and 3F demonstrates the structural preservation of the lamina propria following treatment. For example, histopathology confirms that the PEF treatment as described herein applied at a depth of about 1 mm in duodenal tissue will treat the mucosal layer without the pulsed electric field energy affecting the muscularous propria at a therapeutic level.

In some variations, a pulsed electric field (PEF) treatment may be combined with localized thermal treatment. For example, thermal treatment may be applied to surface tissue or near-surface tissue while PEF treatment may be applied to relatively deeper tissue. As described in more detail herein, the depth of tissue treatment received by one or more layers may be adjusted based on one or more of electrode design, the applied voltage magnitude, time or duration of energy delivery, frequency of applied energy, and tissue configuration. In addition, an expandable member which comprises the electrode array may comprise an outward radial compression force that allows reasonable determination of the location of the electrodes relative to the submucosa and/or muscularis layers.

An example of such control is thermal treatment applied up to a tissue depth of about 0.1 mm and a PEF treatment applied to a tissue depth of up to about 1 mm. The ratio and depth of thermal treatment to PEF treatment may be based on a desired clinical outcome (e.g., effect). In some variations, thermal treatment may be applied up to a tissue depth of about 3 mm, and PEF treatment may be applied up to a tissue depth of about 5 mm. Therefore, in some variations, more thermal treatment than PEF treatment may be applied to tissue. Based on a depth or type of tissue, different healing cascades maybe optimal. In some variations, the villas mucosa at up to about 1 mm may be thermally treated to allow substantially the entire tissue architecture to be replaced, while the submucosa may be PEF treated to preserve the tissue architecture and promote rapid healing of that layer. Furthermore, neither the thermal treatment nor PEF treatment may affect the deeper muscularis propria layer.

FIG. 3B is an image of an illustrative variation of duodenal tissue that has undergone different treatments. In particular, the tissue (360) was treated with pulsed or modulated electric field energy and first mucosa region (362) was further subjected to radiofrequency (RF) ablation energy. The ablated villi of the first mucosa region (362) have broken cellular membranes and destroyed cell structures such that those cells are no longer viable or functioning. By contrast, a second mucosa region (360) has cells that have undergone cell lysis where the cellular membranes remain intact but the cells are no longer viable and functioning. That is, cell lysis corresponds to functional cell death with intact cellular structures while ablation refers to loss of both cell structure and function. The submucosa (370) and muscularis (380) remain healthy (e.g., viable and fully functioning with cell integrity). In FIG. 3B, villi in the first mucosa region (362) are thermally ablated while the cell lysis in the second mucosa region (360) is generated by a pulsed or modulated electric field. A third mucosa region (363) adjacent to the thermal lesion of the first mucosa region (362) is not treated at all and comprises viable tissue.

FIG. 3C illustrates a histological slide of the duodenum from tissue about 24 hours after treatment with heat and pulsed electric field, showing a partial treatment of the mucosa down to the crypt layer, with injured cells. A fourth mucosa region (391) corresponds to thermal/heat fixed tissue of the villi, including the villi-associated enteroendocrine cells. The fourth mucosa region (391) demonstrates architectural and cytological preservation with cellular detail with hyperchromatic nuclear and hypereosinophilic cytoplasmic staining. Overall, interstitial hemorrhage and infiltrating post-treatment-associated inflammatory cells are not identified. The heat fixed tissue may be expected to slough off, followed by surface re-epithelialization and villous structural healing with crypt cell repopulation. The crypt tissues are partially affected by a combination of heat and pulsed electric field effects. The tissue healing timeline is expected to be longer than that of a pulsed electric field treatment without thermal effect. The submucosa (370) and muscularis (380) are histologically unaffected. FIG. 3E is an image of an illustrative variation of 24-hour porcine duodenal histology following an isolated hyperthermic tissue treatment (i.e., no concomitant pulsed electrical field exposure) which destroys the lamina propria in that tissue scaffolding is burned and destroyed and will be sloughed off and removed during healing. This demonstrates the histologic features of a thermal tissue dose, consistent with thermal/heat-induced coagulative necrosis without thermal/heat fixation. In this region, the glandular epithelium and neuroendocrine cells (321) show a loss of cytologic detail, consistent with cellular “ghost images.” Interstitial hemorrhage and reactive inflammatory cells of the mucosal layer (341) are present at the region's edge. The submucosa (331) and muscularis (311) also show injury related changes. This region may be anticipated to heal similar to an ischemic type coagulative necrosis with resorption and remodeling with mucosal regeneration. The thermal lesion destroyed the lamina propria. Scaffolding is burned and destroyed and will be sloughed off and removed during healing. The tissue healing time frame for this region should be longer than that expected for a pulsed electric field treatment.

FIG. 3F is an image of an illustrative variation of duodenal tissue that has undergone treatment with pulsed or modulated electric field energy to a controlled depth not including the muscularis, untreated muscularis propria layer (310), submucosa (330), treated submucosa (332), treated villus crypts, with partial cell lysis and maintained tissue scaffolding (342), and treated villi with villas sloughing (322). The treated submucosa (332) also maintains tissue scaffolding. These treated tissues illustrate cells that have undergone a cell death where the cellular membranes remain intact but the cells are no longer viable and functioning. The healing cascade will replace these cells without infiltration of large number of inflammatory cells, and the surface will re-epithelialize and with villous structural healing and crypt cell repopulation. The muscularis (310) remains healthy (e.g., viable and fully functioning with cell integrity) without therapeutic effect from the pulsed electric field energy. That is, with pulsed or modulated electric field energy cell death corresponds to functional cell death with intact cellular structures while ablation refers to loss of both cell structure and function and an aggressive necrotic inflammatory response healing cascade.

In some variations, a target depth of treatment includes the mucosal layer but excludes treatment of the muscularous propria. Human tissue data assessed through histopathology supports about a 1 mm target depth for PEF tissue treatment where the pulsed electric field does not penetrate through to the muscularous propria at a therapeutic level. Based on the devices, systems and methods described herein, the healing response may be essentially completed in about thirty days. Moreover, the systems, devices, and methods described herein may provide uniform treatment coverage throughout a circumference and length of the duodenum.

Some methods for treating diabetes may include treating the submucosa layer of the duodenum without treating the muscularis. Conventional solutions do not consistently treat the submucosa layer without negatively impacting the muscularis. Instead, conventional solutions may add complicated mitigating steps such as lifts with saline injection in an attempt to protect the muscularis. For reference, the mucosal layer typically has a thickness between about 0.5 mm to about 1 mm, the submucosa layer typically has a thickness of about 0.5 mm and about 1 mm, and the muscularis typically has a thickness of about 0.5 mm. Inducing injury to the muscularis may result in adverse clinical outcomes. Furthermore, the anatomical structure along a circumference of the duodenum is not uniform, thus complicating efforts to treat just the submucosa and not the muscularis.

The methods described herein may selectively change tissue viability without losing the integrity of the majority of the treated tissue by applying a predetermined pulsed or modulated electric field and, optionally, without other treatment of the tissue to mitigate the pulsed or modulated electric field to a portion of tissue. By contrast, RF based energy treatment may predominantly generate heat-induced cell lysis (e.g., cell death) or ablation that may indiscriminately damage tissue and destroy cellular structure, and which may be difficult to modulate, thus negatively impacting treatment outcomes. In some variations, the methods described here may comprise applying a pulsed or modulated electric field to thermally-induce local necrotic cell death (e.g., local ablation) for tissue immediately adjacent to an electrode array and to induce cell lysis (e.g., functional cell death) within a predetermined range of tissue depths of (e.g., up to about 1 mm, between about 0.5 mm and 0.9 mm) while minimizing the physiological impact to tissue greater than the selected depth.

FIG. 3F is an image of an illustrative variation of duodenal tissue that has undergone treatment with pulsed or modulated electric field energy to a controlled depth. In FIG. 3F, the muscularis layer (310) and a portion of the submucosa (330) are untreated (i.e., energy delivered to tissue does not affect the tissue) and the villus crypts (342), villi (322) and a different portion of the submucosa (332) have been treated. Thus, the treatment applied to the duodenal tissue shown in FIG. 3F results in a more superficial (e.g., closer to the tissue surface) treated submucosa (332) and a deeper, untreated muscularis layer (310). The treated tissues contain cells that have undergone cell lysis where the tissue scaffolding remain intact but the cells are no longer viable and functioning. A mild healing cascade will replace these cells. The muscularis (310) adjacent to the treated submucosa (332) remains healthy (e.g., viable and fully functioning with cell integrity).

The pulsed or modulated electric fields near an electrode array may generate some thermal heating of tissue leading to tissue ablation that destroys both cell structure and function. However, cell lysis in tissue resulting from the pulsed or modulated electric fields applied herein are at least 50% pore-induced and less than 50% heat-induced such that a majority of cell death comprises functional cell death with intact cellular structures. For example, the thermal heating generated by a pulsed or modulated electric field is generally localized to a relatively small radius from each electrode of an electrode array and does not affect deeper layers of tissue such as the muscularis.

The systems, devices, and methods described herein may deliver energy to provide treatment characteristics optimized for each tissue layer to improve treatment outcomes. Near the surface of the tissue (e.g., less than about 0.5 mm, between about 0.1 mm and about 0.5 mm), thermal heating may generate local necrotic cell death of tissue that may slough off after treatment. At a tissue depth of between about 0.5 mm and about 1.3 mm (e.g., mucosa of duodenum), cell lysis may be generated by the pulsed or modulated electric field while thermal heating is limited (e.g., to less than about a 13° C. increase or 6° C. increase). For example, an electric field strength at about 1.0 mm may be about 2.5 kV/cm. At tissue depths beyond 1.0 mm, the energy delivered to tissue generates reversible electroporation with even less thermal heating such that deeper tissue may be substantially untreated. Thus, thermal heating may be limited to a surface tissue layer (e.g., less than about 0.5 mm, between about 0.1 mm and about 0.5 mm) while still delivering pulsed or modulated electric field energy for cell lysis of the mucosa.

For example, FIG. 3C is an image of an illustrative variation of duodenal tissue that has undergone a method of treating duodenal tissue described herein where villi (391) has been treated by a combination of thermal heating (e.g., more than 50%) and pore-induced cell death (e.g., less than 50%). The pulsed or modulated electric field applied to the villus crypts and submucosa (370) has treated the tissue to a majority (e.g., more than 50%) of pore-induced cell death with a lesser contribution (e.g., less than 50%) of cell death due to thermal heating. The muscularis (380) is substantially untreated by the pulsed or modulated electric field or other methods. For example, the submucosa in FIG. 3C is not subject to saline injection. The depth of treatment may be controlled such that a predetermined portion of the mucosal layer such as the villus crypts may remain untreated if desired. The configuration and geometry of the electrode arrays as described herein may enable the tissue treatment characteristics described herein.

By contrast, conventional solutions that apply other forms of thermal energy (e.g., steam, radiofrequency, laser, heated liquid) to the duodenum thermally ablate through multiple layers of the tissue (e.g., inducing more than 50% heat-induced necrotic cell death and less than 50% pore-induced cell death), thereby destroying the cellular structure of the mucosa at similar depths and which may detrimentally thermally damage the muscularis. In an attempt to mitigate the risk of unintentional thermal damage during application of thermal energy to deeper layers (e.g., muscularis) of the duodenum, saline may be injected into portions of duodenal tissue (e.g., the submucosa (330)). This additional step further complicates the procedure and is not always sufficient to prevent unwanted thermal tissue damage. The pulsed or modulated electric field based methods described here eliminate this additional step and provide greater protection against unwanted tissue damage by improving the energy delivery characteristics generated by a pulsed electric field device.

In some variations, pulsed electric field treatment may be applied while monitoring and/or minimizing tissue temperature increases. For example, a predetermined rise in tissue temperature (e.g., about 1° C., about 2° C., about 3° C.) may be followed by a pause (e.g., of a predetermined time interval) in energy delivery to allow the tissue to cool. In this manner, the total energy delivered may increase the tissue temperature below a predetermined threshold (e.g., below a safety limit). In some variations, the predetermined threshold may be up to about 3° C., about 6° C., about 10° C., about 13° C., including all ranges and sub-values in-between. In further variations, the predetermined threshold may be between about 1° C. to about 20° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C. In some variations, the pulsed electric field treatment applied to tissue increases a tissue temperature by no more than about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C.

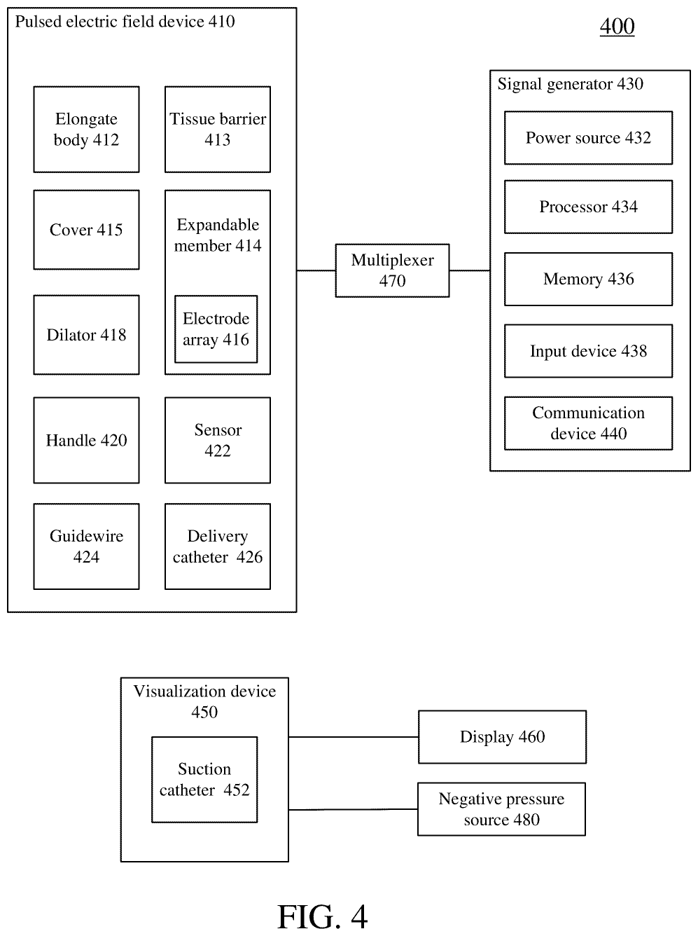

With general reference to FIG. 4, some variations may comprise sensor (422) as a temperature sensor in some variations that is in operative communication with the multiplexor (470) and/or the processor (434) and/or memory (436) of the signal generator (430). The temperature sensor may be configured to sense the temperature of the target tissue. A predetermined upper threshold temperature value may be stored within the memory (436) and/or preprogrammed instructions of the processor (434). If a sensed temperature is obtained that is over the predetermined upper threshold temperature value, some variations may comprise the multiplexor (470) and/or the processor (434) to halt treatment until a sensed temperature is obtained that is below the predetermined upper threshold temperature value.

Moreover, the difficulty faced by conventional solutions in controlling unwanted thermal tissue damage would lead one of ordinary skill away from using the pulsed or modulated electric field energy levels and methods described herein. In some variations, the tissue power densities generated by a pulsed or modulated electric field may be several orders of magnitude higher than the tissue power densities generated by radiofrequency ablation. For example, a power density ratio of an analogous design for radio frequency ablation may be about 576 where a radiofrequency device is driven at about 25 Vᵣₘₛ and a pulsed electric field device is driven at about 600 Vᵣₘₛ. Thus, it would be unexpected for the pulsed or modulated electric field methods described here to not only treat tissue, but to do so without excess thermal tissue damage requiring mitigation procedures. Furthermore, the increased power densities may require additional insulation and protection of the pulsed electric field device, as well as a signal generator capable of generating such peak power levels. Generally, a duty cycle for PEF treatment may be several orders of magnitude lower than radio frequency ablation in order to keep a bulk tissue temperature rise below the predetermined threshold. For example, radio frequency ablation energy may generally be delivered continuously for several seconds. Accordingly, the duty cycle for PEF treatment may be between about 0.0000001 to about 0.001, about 0.000001 to about 0.001, about 0.00001 to about 0.001, about 0.00002 to about 0.001, about 0.00003 to about 0.001, about 0.00003 to about 0.0005, about 0.00003 to about 0.0004, or about 0.000035 to about 0.0004, including about 0.0000001, about 0.000001, about 0.00001, and about 0.00002. For example, in some variations, PEF treatment may collectively accumulate about 5 milliseconds of ON time over about 10 seconds, for a net duty cycle of about 0.0005.

Generally, the devices described here may comprise an elongate body coupled to an electrode array, which may be disposed in a bodily lumen (e.g., a lumen of a duodenum). In some variations, the devices may further comprise an expandable member configured to releasably engage to a portion of the bodily lumen. The expandable member may, when expanded to an expanded configuration produce an outward radial compression force against the target tissue. In addition, the expandable member may comprise or be coupled to an electrode array configured to generate a pulsed or modulated electric field. The electrodes and/or electrode sections of the electrode array may have predetermined dimensions and spacing configured to generate a pulsed or modulated electric field having predetermined uniformity for treating desired tissue while limiting damage to other tissue. In some variations, the expandable member may expand and compress as necessary to engage an inner diameter of the bodily lumen as a result of a force produced by the expandable member that may be directed in a radial direction. In some variations, the contact produced between the electrodes and the target tissue as a result of the outward radial compression force generated by the expanded expandable member may be sufficient. In some variations, additional force may be applied to achieve sufficient electrical contact, e.g., and without limitation, such as may be produced by an exemplary suction device. In some variations, a system comprising the devices described herein may further comprise a signal generator configured to generate a pulse waveform for delivery to the electrode array to thereby treat the engaged tissue.

In some variations, the devices described herein may further comprise a tissue barrier configured to reduce tissue catching with respect to device components such as the elongate body and expandable member, thereby aiding a treatment procedure and preventing undesired tissue damage. For example, the tissue barrier may be coupled between an edge of the expandable member and a corresponding portion of the elongate body. Similarly, the devices described herein may comprise a cover (e.g., tissue shield) configured to reduce tissue catching with respect to the expandable member, thereby facilitating transition of the expandable member between unexpanded (e.g., rolled) and expanded (e.g., unrolled configurations). For example, the cover may be configured to overlap a portion of the expandable member, thus preventing tissue from catching on the portion of the expandable member that extends or retracts radially when transitioning between unexpanded and expanded configurations. By contrast, conventional devices that expand and/or unexpand within a body cavity or lumen of a patient may catch (e.g., trap) tissue at one or more pinch points in a manner that may damage tissue and/or hinder translation of the device within the body cavity.

In some variations, the systems described here may further comprise a suction catheter configured to efficiently provide suction to an expandable member in conjunction with visualization of a treatment procedure. For example, effectively utilizing negative pressure during a procedure may facilitate better contact between the target tissue and the expandable member, but depending on the methods used to apply the negative pressure, may hinder visualization by blocking the field of view and/or requiring visualization device positioning that is sub-optimal or ineffective for the treatment. The systems and devices described herein may provide for improved application of suction or negative pressure, while facilitating visualization of the procedure to ensure safety. As discussed above, some variations may not require a suction catheter to ensure sufficient contact between the electrodes and the target tissue. Instead, these variations may find sufficient contact between the electrodes on an expanded expandable member and the target tissue as a consequence of an outward radial compression force produced by the expanding expandable member.

Also described herein are methods. In some variations, a method of treating tissue to treat a chronic condition, such as, for example, diabetes, may include advancing a pulsed electric field device and a visualization device toward a target tissue. The pulsed electric field device may comprise an expandable member comprising an electrode array. The expandable member may be transitioned from a compressed (e.g., rolled, unexpanded) configuration into an uncompressed (e.g., unrolled, expanded) configuration bringing the expandable member (and the electrode array) closer to or in contact with the surface of the target tissue. The expandable member may comprise a flexibility to apply force against and conform to the target tissue, which, in some instances, may have a non-uniform surface and/or size. For example, the expandable member may comprise a flexibility to apply force against and conform to an inner circumference of the duodenum, which may itself comprise a range of diameters. In some variations, a visualization device may be configured to visualize one or more of the pulsed electric field device and all or a portion of the target tissue and/or surrounding tissue. A suction catheter may be advanced from a lumen of the visualization device toward a lumen of the expandable member in an expanded configuration. Suction may be applied to the target tissue through the expandable member using the suction catheter.

A first pulsed electric field waveform may be delivered to the electrode array to generate a first pulsed or modulated electric field, which may treat a first portion of target tissue. In some variations, the electrode array may have a plurality of sections. A first pulsed electric field waveform may be delivered to two or more non-proximate (e.g., non-adjacent, not immediately next to each other) sections of the plurality of sections in a predetermined sequence, which may increase safety and/or reduce unintended damage to the tissue by reducing a temperature increase in tissue. In some variations, the pulsed electric field device may be moved (e.g., advanced or retracted) toward a second portion of the target tissue (which may be distal or proximal to the first portion of the target tissue), and a second pulsed electric field waveform may be delivered to the electrode array to generate a second pulsed or modulated electric field thereby treating the tissue in the second portion. For example, in some variations, a signal generator may generate a drive voltage (e.g., voltage measured at an electrode array) of between about 400 V and about 1500 V that may correspond to an electric field strength of about 400 V/cm and about 7000 V/cm at the treatment portions of the duodenum. The expandable member may be in a compressed configuration, semi-expanded configuration, or an expanded configuration during movement of the pulsed electric field device. In some variations, sensor measurements (e.g., temperature, impedance) may be used to monitor and/or control pulse waveform delivery. In some variations, current and voltage measurements may be used to monitor and/or control pulse waveform delivery.

### I. System

Systems described here may include one or more of the components used to treat tissue, such as, for example, a pulsed electric field device and a visualization device. Suitable examples of such systems and devices are described in International Application Serial No. PCT/US2022/025630, filed on Apr. 20, 2022, the disclosure of which is hereby incorporated by reference in its entirety. FIG. 4 is a block diagram of a variation of a pulsed electric field system (400) comprising one or more of a pulsed electric field device (410), a pulse or signal generator (430), multiplexer (470), a visualization device (450), and a display (460).

In some variations, the pulsed electric field device (410) may comprise one or more (e.g., a first and a second) elongate bodies (412) sized and shaped to be placed in one or more body cavities or lumens of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. In some variations, the pulsed electric field device (410) may further comprise one or more tissue barriers (413), one or more expandable members (414), one or more covers (415), one or more electrode arrays (416), one or more dilators (418), a handle (420), one or more sensors (422), a guidewire (424), and a delivery catheter (426). A distal end of the pulsed electric field device (410) may comprise the dilator (418), and the guidewire (424) may extend from a lumen of the dilator (418). The expandable member (414) may comprise the electrode array (416). For example, as will be described in more detail herein, in some variations the electrode array (416) may be coupled to a surface (e.g., outer surface) of the expandable member (416), while in other variations, the electrode array itself may form the expandable member and/or the electrode array may be integral with the expandable member. In some variations, the electrode array may have a plurality of sections that may be energized individually (e.g., concurrently, consecutively) to treat tissue in a predetermined sequence as described in more detail herein.

In some variations, the expandable member (414) and/or the electrode array (416) may be disposed adjacent to one or more dilators, for example, between at least a pair of dilators (418). In some variations, the tissue barrier (413) and the cover (415) may each be configured to reduce tissue catching with the pulsed electric field device (410) as further described herein. In some variations, the pulsed electric field system (400) may optionally comprise a delivery catheter (426) configured to advance over the pulsed electric field device (410). Additionally or alternatively, the pulsed electric field device (410) may comprise one or more sensors (422) configured to measure one or more predetermined characteristics such as temperature, pressure, impedance and the like.

As mentioned above, the pulsed electric field system (400) may comprise a visualization or imaging device (450). In some variations, the visualization device (450) may be configured to visualize one or more steps of a treatment procedure. The visualization device (450) may aid one or more of advancement of the pulsed electric field device (410), positioning of the pulsed electric field device and/or components thereof (e.g., the electrode array (416)), and confirmation of the treatment procedure. For example, the visualization device (450) may be configured to generate an image signal that is transmitted to a display (460) or output device. In some variations, the visualization device (450) may be advanced separately from and alongside the pulsed electric field device (410) during the treatment procedure. For example, an expandable member (414) of the pulsed electric field device (410) may be configured to hold the visualization device (450) such that the pulsed electric field device (410) translates together with the visualization device (450) as they are moved through the body. The expandable member (414) may expand to release the visualization device (450), thus allowing freedom of movement for the visualization device (450). In other variations, the visualization device (450) may be integrated with the pulsed electric field device (450). For example, the dilator (418) may comprise the visualization device (450).

The visualization or imaging device (450) may be any device (internal or external to the body) that assists a user in visualizing a treatment procedure. In some variations, the visualization device (450) may comprise one or more of an endoscope (e.g., chip-on-the-tip camera endoscope, three camera endoscope), image sensor (e.g., CMOS or CCD array with or without a color filter array and associated processing circuitry), camera, fiberscope, external light source, and ultrasonic catheter. In some variations, an external light source (e.g., laser, LED, lamp, or the like) may generate light that may be carried by fiber optic cables. Additionally or alternatively, the visualization device (450) may comprise one or more LEDs to provide illumination. For example, the visualization device (450) may comprise a bundle of flexible optical fibers (e.g., a fiberscope). The bundle of fiber optic cables or fiberscope may be configured to receive and propagate light from an external light source. The fiberscope may comprise an image sensor configured to receive reflected light from the tissue and the pulsed electric field device. It should be appreciated that the visualization or imaging device (450) may comprise any device or devices that allows for or facilitates visualization of any portion of the pulsed electric field device and/or of the internal structures of the body. For example, the visualization device may comprise a capacitive sensor array and/or a fluoroscopic technique for real-time X-ray imaging.

In some variations, the system may comprise a suction catheter (452) configured to apply suction to the expandable member (414) and tissue. For example, the suction catheter (452) may be slidably positioning within, and advanced from, a lumen of the visualization device (450). In some variations, the suction catheter (452) may be advanced from the lumen of the visualization device (450) to or into a lumen of the expandable member (414) in an expanded configuration while the visualization device (450) is positioned proximally of the expandable member (414). In some variations, the suction catheter (452) may be fluidically coupled to a negative pressure source (480).

Generally, a voltage pulse or signal generator (430) may be configured to provide energy (e.g., energy waveforms, pulse waveforms) to the pulsed electric field device (410) to treat predetermined portions of tissue, such as, for example, duodenal tissue. In some variations, a PEF system as described herein may include a voltage pulse or signal generator (430) that comprises an energy source and a processor. The signal generator (430) may be configured to deliver a bipolar waveform to an electrode array (416), which may deliver energy to the target tissue (e.g., duodenal tissue). The delivered energy may aid in resurfacing or otherwise treating the target tissue at a desired treatment depth while minimizing damage to surrounding tissue. In variations in which the desired tissue is duodenal tissue, the delivered energy may aid in resurfacing the mucosa of the duodenum while minimizing damage to surrounding tissue (e.g., muscularis tissue). In some variations, the signal generator (430) may generate one or more bipolar waveforms.

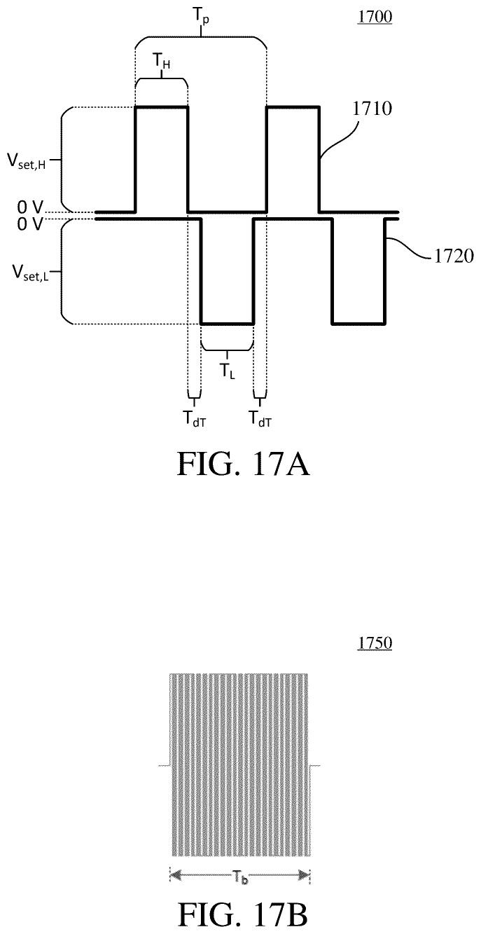

In some variations, in order to limit nerve stimulation, a pulse waveform may, on average, comprise a net current of about zero (e.g., generally balanced positive and negative current) as illustrated in the variation of FIG. 17A. In some variations, a slight net current imbalance may be present (either positive or negative) while still not stimulating nerves. In some variations, a pulse waveform may have a non-zero time or pulse width of less than about 2 μsec or less than about 5 μsec, though other non-zero times or pulse widths are certainly possible and within the scope of the present disclosure. In some variations, a pulse waveform may comprise a square or rectangular waveform. For example, the pulse waveform may comprise a square or rectangular shape in voltage drive and in current drive, or the pulse waveform may comprise a square or rectangular shape in voltage drive and a sawtooth shape in current drive. In some variations, one or more pulses may comprise a half sine wave for both current and voltage. In some variations, one or more pulses may comprise two exponentials with different rise and fall times. In some variations, one or more pulses may comprise bipolar pulse at a first voltage potential followed by one or more pulses at a second potential less than the first potential. In some variations, the generated pulse waveforms may be substantially identical, while in other variations, one or more of the generated pulse waveforms may be different from the remaining pulse waveforms.

As shown in FIG. 4, the voltage pulse or signal generator (430) may comprise a power source (432) operatively configured to power the signal generator (430), a processor (434) configured to execute preprogrammed or instructions entered via an input device (438), a memory (436) in operative communication with the processor (434) and configured to store preprogrammed instructions and other data, including but not limited to any sensed data obtained during the subject procedure, and a communication device 440 in operative communication with at least the processor (434).

In some variations, a multiplexer (470) may be operatively coupled to the pulsed electric field device (410). For example, the multiplexer (470) may be coupled between the signal generator (430) and the pulsed electric field device (410), or the signal generator (430) may comprise the multiplexer (470). The multiplexer (470) and/or preprogrammed instructions within the processor (434) may be configured to operative select a subset of electrodes or electrode sections of an electrode array (416) receiving a pulse waveform generated by the signal generator (430) according to a predetermined sequence. For example, in some variations, the electrode array (416) may comprise one or more electrode sections that correspond to a subset of electrodes. The electrode array (416) may comprise between 1 and 10 electrode sections, including 1, 2, 3, 4, 5, 6, 7, 8, 9, 10, or more sections. Each electrode section may comprise the same number of electrodes and/or the same surface area as every other section, but need not. The predetermined sequence may be optimized to treat tissue at a given treatment site. Additionally or alternatively, the multiplexer (470) may be coupled to a plurality of signal generators and may be configured to select between a waveform generated by one of the plurality of signal generators (430) for a selected subset of electrodes. In the variations described above, the electrode sections of the electrode array (416) may be electrically insulated from each other so that the multiplexer (470) drives the delivery of pulsed electric field waveforms to each individual electrode section.

In other variations, two or more of the electrode sections of the electrode array (416) may be electrically and operatively connected such that a generated pulse waveform received by the electrode array results in a pattern of pulsed electric field generation that is predetermined based on the electrical interconnection pattern between the two or more electrode sections.

In other variations, a combination of the multiplexer (470) and an electrical interconnection between two or more electrode sections of the electrode array (416) may be provided. In these variations, the multiplexer (470) may operatively deliver a pulsed electric field waveform to electrically interconnected electrodes sections.

In some variations, the multiplexer (470) and the signal generator (430) may be configured to deliver a pulsed electric field waveform to two or more non-proximate electrode sections (e.g., first section, second section) of the plurality of electrode sections in a predetermined sequence. For example, the predetermined sequence may comprise activating a first electrode section followed by a second electrode section after a predetermined and/or preprogrammed inter-section delay where activation of each of the first and second electrode sections generates a therapeutic electric field, and where the first and second electrode sections are not adjacent (i.e., directly next to) one another. Stated differently, the predetermined sequence may comprise activating a first electrode section followed by a second section, where at least a third section is positioned between the first and second electrode sections. Other variations may comprise activating at least some electrode sections that are adjacent. In some variations, the electrode sections may be activated in a “sweeping” pattern, wherein adjacent electrode sections are successively activated, interposed by a predetermined inter-section delay, such that the generated therapeutic field effectively sweeps around the target tissue.

As another example, and without limitation, the signal generator (430) may be configured to deliver a series of bipolar pulses to two or more non-proximate sections of the plurality of sections in a predetermined sequence for a cumulative activation time of between about 0.1 ms and about 10 ms over a treatment period between about 30 seconds and about 35 seconds. Each bipolar pulse may comprise a pulse width between about 1 μs and about 10 μs, and the electrode array may be configured to deliver between about 0.05 J per bipolar pulse and about 0.5 J per bipolar pulse and an instantaneous power between about 26,000 W per bipolar pulse and about 70,000 W per bipolar pulse. In some variations, the predetermined sequence may comprise a duty cycle between about 0.003% and about 0.004%, including all ranges and sub-values in-between. In some variations, the signal generator may be configured to control waveform generation and delivery in response to received sensor data. For example, energy delivery may be modulated (e.g., inhibited) based on one or more of a measured temperature and impedance.

In some variations, the amount of energy delivered by the electrode sections may be about the same throughout execution of the predetermined sequence of pulses and resultant waveforms. In other variations, the amount of energy delivered by the electrode sections may vary throughout the predetermined sequence. In some variations, the pulse waveform may be about the same throughout the execution of the predetermined sequence of pulses and resultant waveforms. For example, and without limitation, pulse widths may be the same in some variations while pulse widths may vary in other variations across the predetermined sequence of pulses and resultant waveforms. Moreover, in some variations, the pulses and resultant waveforms generated by a predetermined sequence of pulses may be substantially the same. In other variations, one or more of the pulses and resultant waveforms generated by the predetermined sequence of pulses may be different than one or more of the remaining pulses and resultant waveforms.

### Pulsed Electric Field Device

Generally, and with continued reference to FIG. 4, the pulsed electric field devices described herein may comprise an elongate body and an expandable member comprising an electrode array. The pulsed electric field devices may be configured to facilitate deployment in, and treatment of target tissue such as tissue within a body cavity or lumen such as the duodenum. For example, the pulsed electric field device may be configured to apply pulsed or modulated electric field energy to an inner surface or circumference of the body cavity or lumen. The devices, systems and methods described herein may be used to treat only a particular, pre-specified portion of a body cavity or lumen (e.g., duodenum), and/or an entirety of the body cavity or lumen (e.g., the entire length of the duodenum).

Additionally or alternatively, the devices, systems and methods described herein may be used to treat one or more of a duodenum, a pylorus, an esophagus, a stomach, a small intestine, and a large intestine (e.g., cecum, colon, rectum, anal canal), as well as any body cavity or lumen of the patient such as vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. The treated tissue may treat one or more of a metabolic disorder, pre-cancer, cancer, proinflammatory processes, immunological processes, Alzheimer's disease, and neurological disorders. For example, the metabolic disorder may comprise one or more of obesity, non-alcoholic fatty liver disease (NAFLD), nonalcoholic steatohepatitis (NASH), Type I diabetes, and Type II diabetes.

In some variations, an electrode array of the pulsed electric field device may generate an electric field strength of from about 400 V/cm to about 1500 V/cm, from about 1500 V/cm to about 4500 V/cm, including all values and sub-ranges in-between, at a treatment depth of from about 0.5 mm to about 1.5 mm from an inner surface of the duodenum, for example, at about 1 mm. For example, in some variations, the pulsed electric field may comprise an electric field strength (i.e., magnitude) between about 2000 V/cm to about 4500 V/cm, about 3000 V/cm to about 4500 V/cm, about 3500 V/cm to about 4500 V/cm, about 3750 V/cm to about 4250 V/cm, or about 3900 V/cm to about 4100 V/cm, including about 2000 V/cm, about 3000 V/cm, about 3500 V/cm, about 3750 V/cm, about 3900 V/cm, about 4000 V/cm, about 4100 V/cm, about 4250 V/cm, and about 4500 V/cm.

In some variations, the electric field may decay such that the electric field strength is less than about 400 V/cm at about 3 mm from the inner surface of the duodenum. In some variations, a predetermined bipolar current and voltage sequence may be applied to an electrode array of the pulsed electric field device to generate the pulsed or modulated electric field. The generated pulsed or modulated electric field may be substantially uniform to robustly induce cell lysis in a predetermined portion of duodenal tissue. For example, a generated pulsed or modulated electric field may spatially vary up to about 20% at a predetermined depth of tissue, between about 5% and about 20%, between about 10% and 20%, and between about 5% and about 15%, including all ranges and sub-values in-between. Furthermore, the pulsed electric field device may be biocompatible and resistant to stomach acids and intestinal fluids.

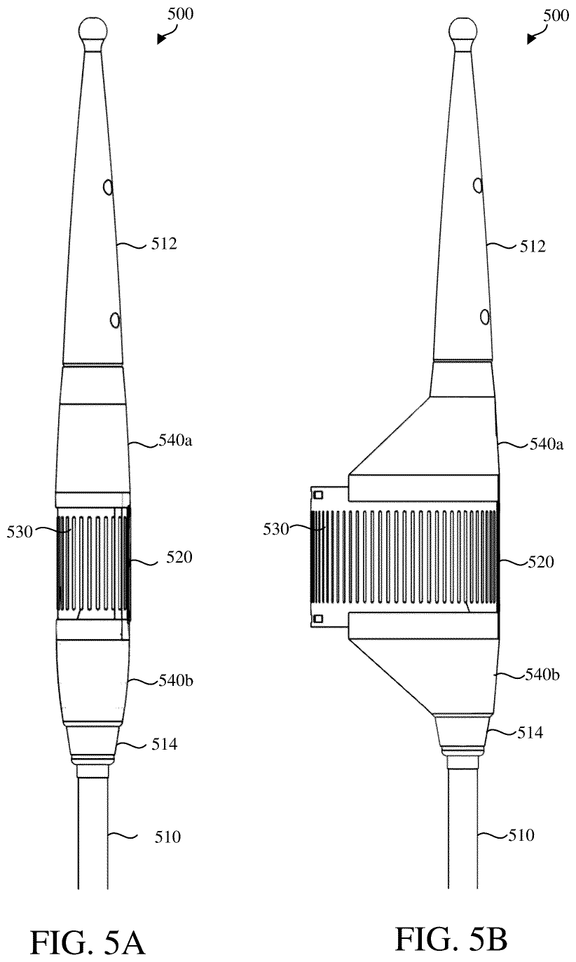

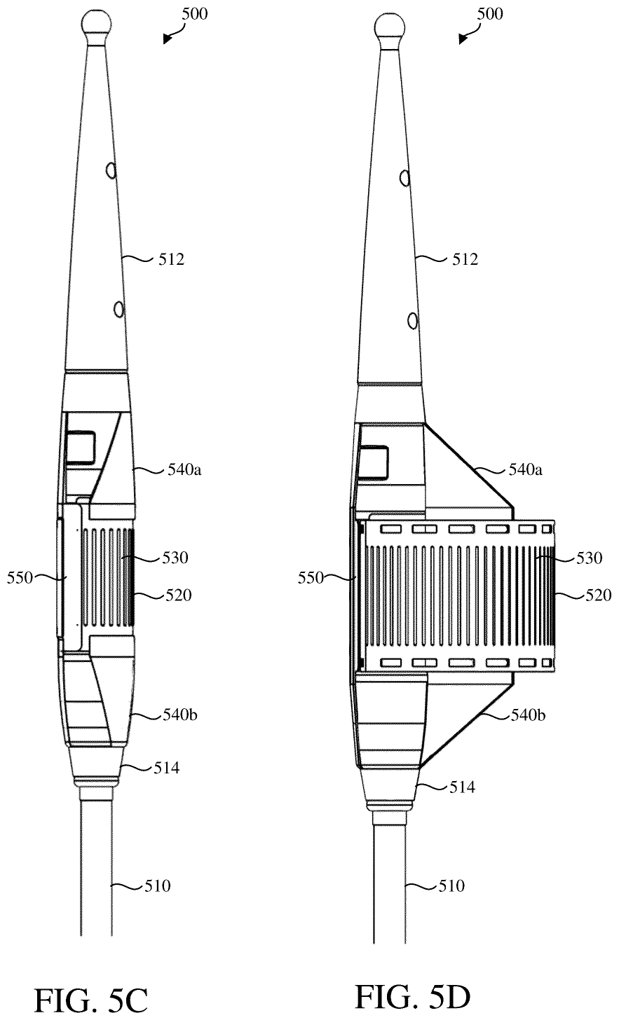

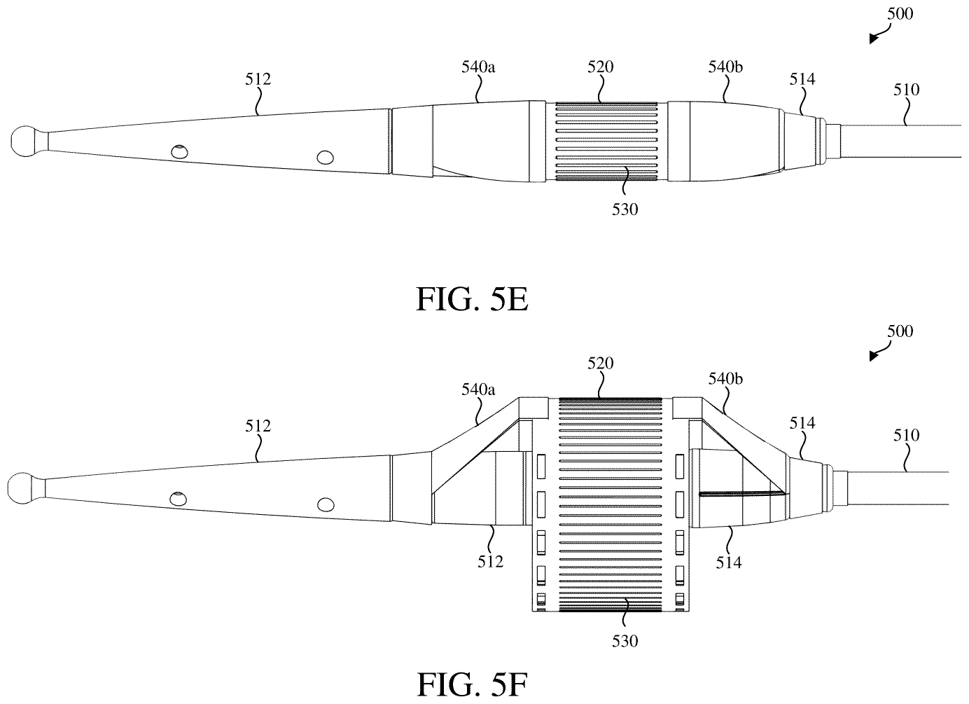

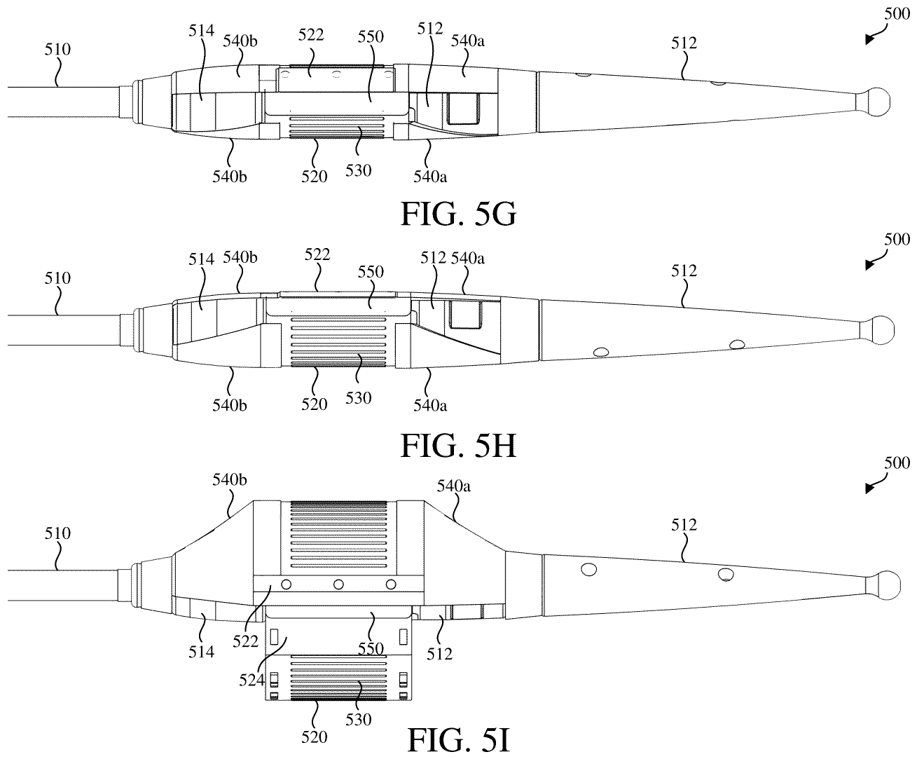

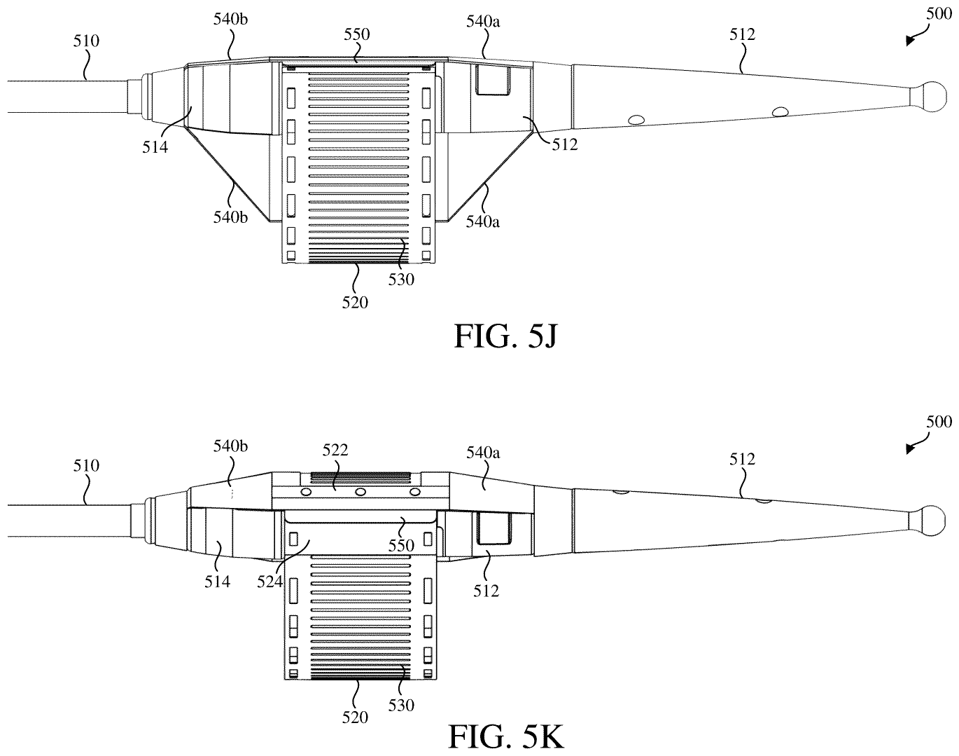

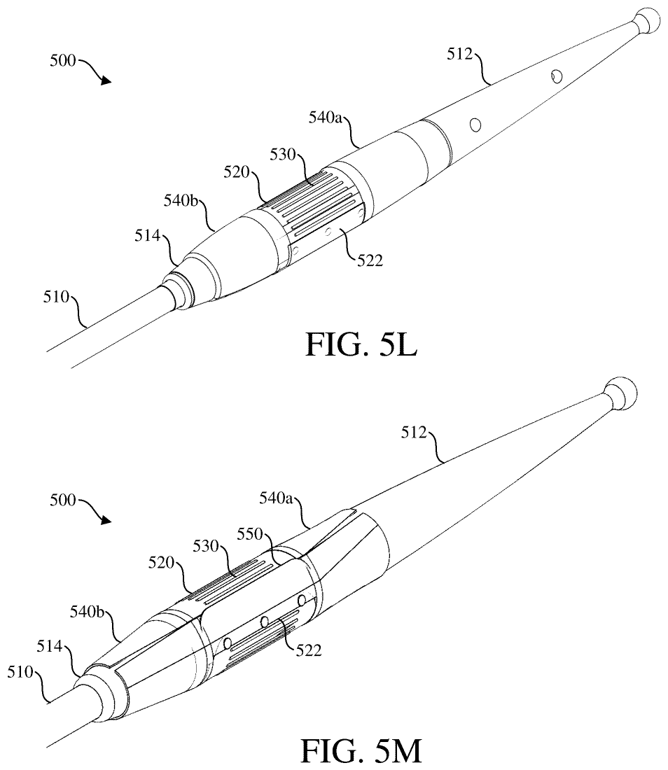

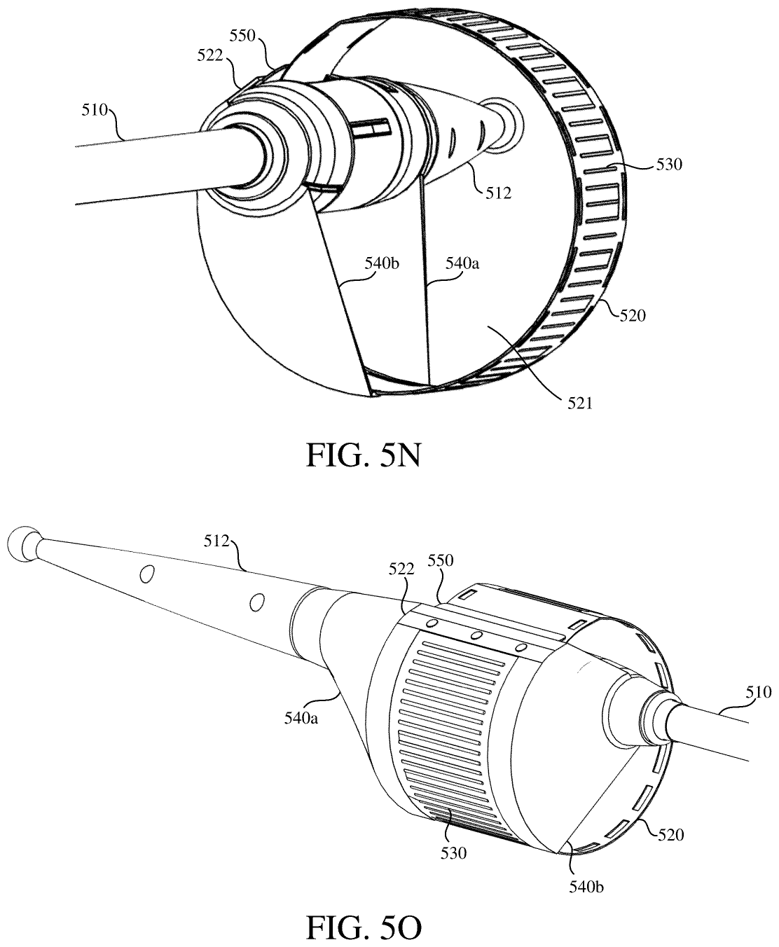

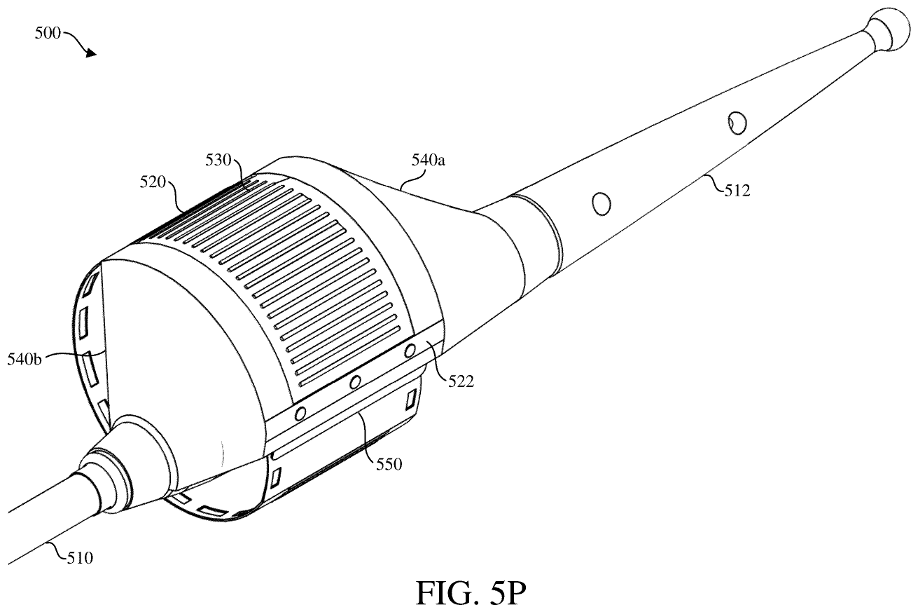

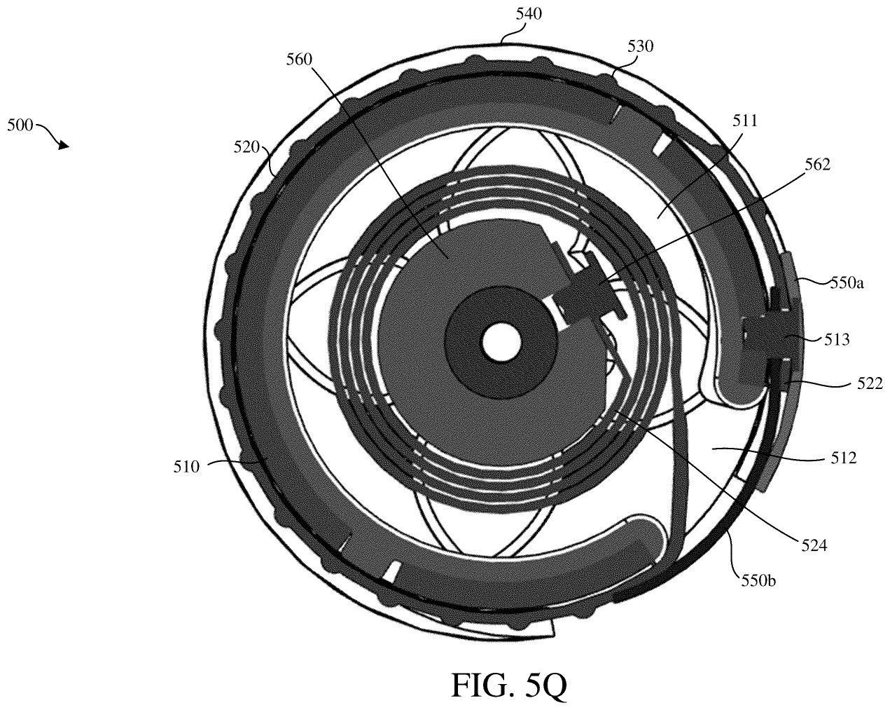

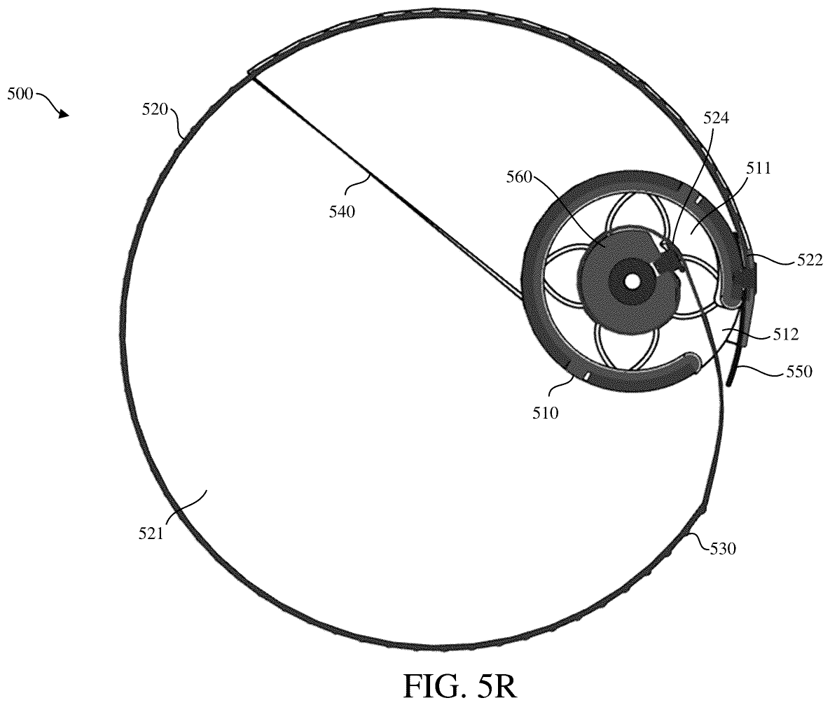

FIGS. 5A-5R are various schematic views of an illustrative variation of a pulsed electric field device (500) in respective unexpanded and expanded configurations. As depicted there, the pulsed electric field device (500) may comprise a first elongate body (510) comprising a lumen (511) therethrough and an expandable member (520).

FIG. 5A shows the expandable member (520) in an unexpanded (e.g., rolled, compressed) configuration and FIG. 5B shows the expandable member (520) in an expanded (e.g., unrolled, uncompressed) configuration. In some variations, the first elongate body (510) may comprise a distal portion (512) and a proximal portion (514). The expandable member (520) may comprise an electrode array (520), and inner end (524) coupled to a second elongate body (560), and an outer end (522) coupled to the first elongate body (510). The expandable member (520) may be disposed between the proximal portion (514) and the distal portion (512). In some variations, the proximal portion (514) may comprise a proximal dilator and the distal portion (512) may comprise a distal dilator.

The pulsed electric field device (500) may further comprise a tissue barrier (540) configured to provide a smoother surface along a longitudinal axis of the device (500) and adjacent to the expandable member 520 to prevent undesirable tissue engagement (e.g., catching) with the device (500) that would pinch tissue and/or potentially hold the device (500) in place relative to the tissue. For example, the tissue barrier (540) may be configured to prevent tissue from becoming trapped between the expandable member (520) and a distal edge of the proximal portion (514) of the first elongate body (510) and/or a proximal edge of the distal portion (512) of the first elongate body (510). In some variations, the tissue barrier (540) may comprise a first portion (540a) (e.g., distal portion) and a second portion (540b) (e.g., proximal portion). In some variations, the first portion (540a) may be coupled to a distal edge of the expandable member (520) and to the distal portion (512) of the first elongate body (510). The second portion (540b) may be coupled to the proximal edge of the expandable member (520) and to the proximal portion (514) of the first elongate body (510).

In some variations, the first and second portions (540a, 540b) of the tissue barrier (540) may comprise a compressed, or rolled or folded, configuration and may achieve an expanded, or unrolled of folded, configuration. The tissue barrier (540) in some variations is coupled to the distal and proximal edges of the expandable member (520) and may comprise a compressed or expanded configuration to accommodate compression or collapsing as well as expansion of the expandable member (520).

The pulsed electric field device (500) may further comprise a cover (550) configured to reduce tissue catching between the expandable member (520) and the first elongate body (510). In some variations, the cover (550) may be coupled between the outer end (522) of the expandable member (520) and the first elongate body (510). The cover (550) may be configured to overlap a portion of the expandable member (520). For example, FIGS. 5Q and 5R illustrate an opening 512 of the first elongate body (510) through which the expandable member (520) transitions between unexpanded and expanded configurations. When the expandable member (520) transitions from the expanded configuration to the unexpanded configuration, the expandable member (520) retracts into a lumen (511) of the first elongate body (510) and rolls about the second elongate body (560). Any tissue engaged with the expandable member (520) during this transition may also be drawn into the lumen (511) and may be caught between the expandable member (520) and the first elongate body (510). However, the cover (550) may be configured to separate tissue from the expandable member (520) before being caught between the expandable member (520) and the first elongate body (510), thereby facilitating transition of the expandable member (520) between rolled and unrolled configurations.

### Expandable Member

Generally, the expandable member variations described herein may be configured to change configurations to aid in positioning of the electrode array relative to target tissue during a treatment procedure. For example, the expandable member (e.g., circuit substrate, flex circuit) may expand to contact tissue to hold the pulsed electric field device in place (e.g., elongate body, electrode array, sensor) relative to the tissue. The expandable member may also partially expand to hold a visualization device in place relative to the pulsed electric field device. The expandable member may comprise a compressed configuration and an expanded configuration. As will be discussed in more detail herein, in some instances, the compressed configuration may be a rolled configuration and the expanded configuration may be an unrolled configuration. Moreover, in some variations, the expandable member may comprise a semi-expanded (or partially unrolled) configuration between the compressed configuration and the expanded configuration. Placing the expandable member in the compressed configuration may allow the pulsed electric field device to be compact in size, which may allow for easier advancement through one or more body cavities. Once appropriately positioned, the expandable member may be transitioned to the expanded configuration, which may allow an electrode array of the expandable member to contact or better contact a tissue surface, such as, for example, all or a portion of an inner circumference of the duodenum or other body lumen. In some variations, the semi-expanded configuration may allow the expandable member to hold another device (e.g., visualization device) within a lumen of the expandable member. Additionally or alternatively, a lumen having one or more openings, apertures, holes, slots, combinations thereof, and the like.

In some variations, the expandable member (520) may be rolled around (e.g., wound about, spooled) and/or in contact with a second elongate body (560) about a longitudinal axis thereof. For example, as shown in the cross-sectional views of FIGS. 5Q and 5R, a second elongate body (560) may be at least partially positioned within a lumen (511) of the first elongate body (510). Furthermore, the expandable member (520) may comprise a plurality of turns such that the expandable member (520) may be rolled about the second elongate body (560) where the expandable member (520) forms a plurality (e.g., two, three, four, five, or more) layers wrapped around or rolled about the second elongate body (560). That is, the expandable member (520) may be in mechanical contact with the second elongate body (560). FIG. 5Q shows the expandable member (520) in an unexpanded or rolled configuration and FIG. 5R shows the expandable member (520) in an expanded or unrolled configuration.

In some variations, the expandable member (520) may comprise an electrode array (530), which may comprise any of the electrode arrays described herein. For example, in some variations, the expandable member (520) may be a flex circuit, while in other variations, the expandable member (520) may comprise a base layer and a flex circuit comprising an electrode array may be coupled to the base layer. The electrode array (530) may be disposed on an outer surface of the expandable member (520) facing away from the second elongate body (560).

FIGS. 5A, 5C, 5E, 5G, 5H, 5L, 5M, and 5Q depict a pulsed electric field device (500) with the expandable member (520) in an unexpanded or rolled configuration configured for advancement through one or more body cavities. When in the unexpanded or rolled configuration, the expandable member (520) may have a generally cylindrical shape (e.g., conforming to the shape of the first elongate body (510)) with a first inner diameter (e.g., lumen diameter) and a first outer diameter. FIGS. 5B, 5D, 5F, 5I-5K, 5N, 5O, and 5R depict the pulsed electric field device (500) with the expandable member (520) in an expanded or unrolled configuration configured for engagement with tissue, such as an inner surface of a duodenum (not shown for the sake of clarity). In some variations, the first elongate body (510) may have a diameter of between about 5 mm and about 10 mm, an inner diameter of up to about 1.3 mm, and a length between about 1500 and about 1900 mm, and between about 1800 and about 1900 mm, including all ranges and sub-values in-between. When in the expanded or unrolled configuration, the expandable member (520) may have a generally elliptic or cylindrical shape with a second inner diameter and a second outer diameter having a predetermined diameter larger than a respective first inner diameter and first outer diameter. The expandable member (520) in the expanded configuration may have a predetermined flexibility configured to conform to a shape of the tissue to which it is engaged.

In some variations, the first and second elongate bodies (510, 560) may be configured to axially rotate relative to one another to transition the expandable member (520) between the unexpanded configuration, the expanded configuration, and a semi-expanded configuration therebetween. For example, the second elongate body (560) (e.g., inner torsion member, rotatable member) may be rotatably positioned within a lumen of the first elongate body (510), such that rotation of the second elongate body (560) relative to the first elongate body (510) (or rotation of the first elongate body (510) relative to the second elongate body (560)) may transition the expandable member (520) between a rolled configuration and an unrolled configuration. As described in more detail herein, a suction catheter, such as a suction catheter advanced from a visualization device (not shown), may be disposed within the lumen (521) of the expandable member (520) to aid in tissue engagement. For example, the suction catheter may be configured to apply suction through the lumen (521) of the expandable member (520). It should be appreciated that the pulsed electric field device (500) may be advanced next to a visualization device and/or over a guidewire. In some variations, a visualization device may be used to guide advancement and to visualize a treatment procedure such that a guidewire and/or other visualization modalities (e.g., fluoroscopy) are not needed.

In some variations, the expandable member (520) may be configured to transition to a configuration between the unexpanded and expanded configurations. For example, the expandable member (520) may transition to a partially or semi-expanded configuration (between the compressed configuration and expanded configuration) that may allow a visualization device (e.g., endoscope) to be disposed within a lumen of the expandable member (520). In some variations, an inner surface of the expandable member (520) may engage and hold a visualization device in a semi-expanded configuration.

In some variations, the second elongate body (560) may be in operative rotatable communication with the handle (420), wherein rotation of at least a portion of the handle (420) also rotates the second elongate body (560) relative to the first elongate body (510). Rotating the second elongate body (560) in a first rotational direction may result in an expansion, e.g., unrolling, of the expandable member (520) and rotating the second elongate body (560) in a second rotational direction that is opposite of the first rotational direction may result in compressing, e.g., rolling, the expandable member around the second elongate body (560).

As shown in the cross-sectional views of FIGS. 5Q and 5R, the expandable member (520) may comprise an inner end (524) (e.g., innermost portion of roll) and an outer end (522) (e.g., outermost portion of roll). FIG. 5Q depicts the expandable member (520) in the unexpanded configuration where the expandable member (520) is rolled about both the first and second elongate bodies (510, 560). For example, the expandable member (520) rolls about the second elongate body (560) for a plurality of turns and at least an outer end (522) of the expandable member (520) engages with the outer surface of the first elongate body (510) so as to minimize a diameter of the pulsed electric field device (500) and facilitate translation of the device (500) through one or more body cavities of the patient. In some variations, the inner end (524) of the expandable member (520) may be coupled to the second elongate body (560) through the second connector (562). Optionally, one or more electrical leads may be coupled to the electrode array (530) through the second elongate body (560) and second connector (562) and configured to conduct electrical pulse energy generated by the pulse or signal generator 430 discussed supra.

FIG. 5R depicts the expandable member (520) in the expanded configuration where the expandable member (520) is unrolled from the second elongate body (560) such that the expandable member defines a lumen (521). The expandable member (520) in the expanded configuration may have a predetermined flexibility configured to conform to a shape of the tissue to which it is engaged.

In some variations, the inner end (524) may be coupled to the second elongate body (e.g., attached to an external surface thereof) (560) and the outer end (522) may be coupled to the first elongate body (510) (e.g., an external surface thereof). Coupling the ends of the expandable member (520) to the first and second elongate bodies (510, 560) in this way allows for better control over the size and shape of the expandable member (520). For example, the inner end (524) may be attached to an outer surface of the second elongate body (560) such that the inner end (524) rotates with the rotation of the second elongate body (560). A direction of the rotation (e.g., clockwise, counter-clockwise) of the second elongate body (560) may determine the configuration (e.g., rolled, unrolled) of the expandable member (520). For example, rotating the second elongate body (560) in a clockwise direction relative to the first elongate body (510) may expand or unroll the expandable member (520), while rotating the second elongate body (560) in a counter-clockwise direction relative to the first elongate body (510) may compress or roll the expandable member, or vice versa. A portion of the first elongate body (510) may comprise an opening (e.g., slit) configured to facilitate transition of the expandable member (520) between unexpanded and expanded configurations.

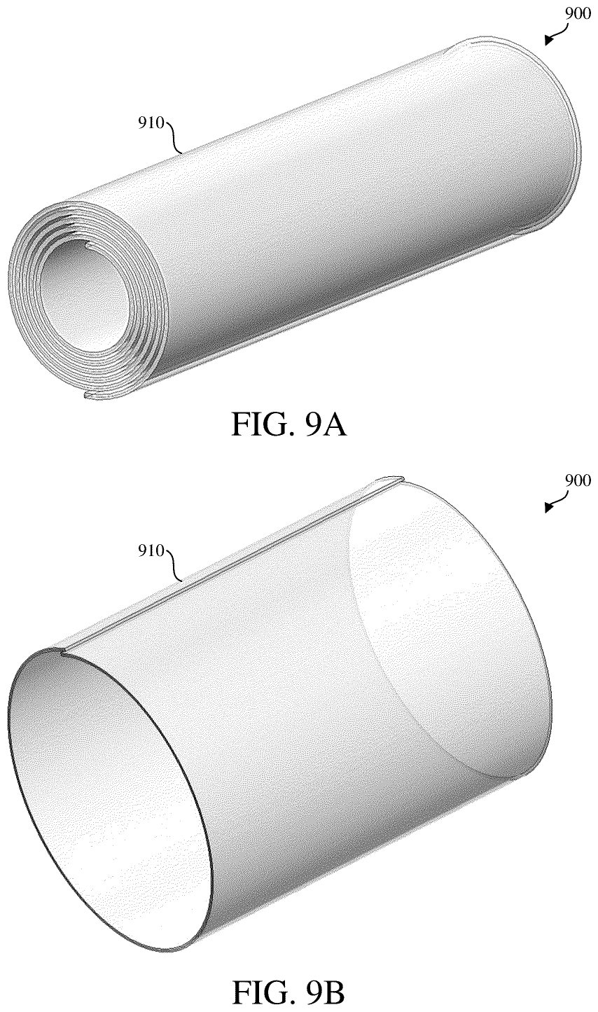

FIG. 9A is a perspective view of a variation of an expandable member (900) in a rolled configuration and FIG. 9B is a perspective view of the expandable member (900) in an unrolled configuration. In some variations, the expandable member (900) may comprise a substrate (910) such as a flex circuit. Furthermore, the expandable member (900) may comprise or be coupled to an electrode array (not shown). In some variations, the expandable member (900) may be composed of a self-expanding material biased to expand to a predetermined shape and/or diameter to assist in achieving sufficient apposition between the electrodes of the expandable member and the target tissue. For example, the expandable member (900) may comprise one or more of a flexible polymeric material (e.g., polyamide, PET), nitinol, stainless steel, copper, gold, other metals, adhesives, combinations thereof, and the like. In some variations, the expansion and compression of an expandable member (900) may be caused by respective retraction and advancement of a sheath (e.g., delivery catheter) over the expandable member (900). The expandable member in the rolled configuration may comprise one or more turns. In some variations, the expandable member (900) in the rolled configuration may have a diameter between about 6 mm and about 25 mm, between about 10 mm and about 25 mm, and between about 15 mm and about 20 mm, including all ranges and sub-values in-between. In some variations, the expandable member (900) in the expanded configuration may have a diameter between about 10 mm and about 60 mm, between about 20 mm and about 50 mm, between about 30 mm and about 50 mm, and between about 40 mm and about 50 mm, including all ranges and sub-values in-between. In some variations, the expandable member (900) may have a width of at least 10 mm, between about 10 mm and about 60 mm, between about 10 mm and about 50 mm, between about 10 mm and about 30 mm, and between about 20 mm and about 40 mm, including all ranges and sub-values in-between.

In some variations, the expandable member may comprise one or more sensors configured to determine a configuration of the expandable member. For example, the sensor may comprise one or more inductive coils configured to measure proximity between the expandable member and the second elongate body such that a length of the unrolled expandable member may be determined. For example, one or more sensors may be disposed along a length of the expandable member. In some variations, a configuration of the expandable member may be confirmed via visual confirmation and via the sensor data.

Additionally or alternatively, the pulsed electric field device (500) may comprise a second expandable member (e.g., inflatable member, balloon, support, basket, frame, cage) (not shown) disposed distal to the expandable member (520). For example, the balloon can be inflated during use with any inert fluid, such as saline, contrast fluid, air, combinations thereof and the like.

### Electrode Array

Generally, the electrodes and electrode arrays described herein may be configured to treat tissue, such as the duodenal tissue, of a patient. In some variations, the electrode array may engage the tissue and be energized to treat a predetermined portion of tissue to resurface the or otherwise treat the tissue. For example, tissue may undergo cell lysis using PEF energy during a treatment procedure. PEF energy tissue treatment may be uniformly delivered at a predetermined depth (e.g., about 1 mm) to quickly and precisely treat tissue without significant damage to surrounding (e.g., deeper) tissue.

In some variations, tissue treatment characteristics may be controlled by the size, shape, spacing, composition, and/or geometry of the electrode array. For example, the electrode array may be flexible to conform to non-planar tissue surfaces. In some variations, the electrode array may be embossed or reflowed to form a non-planar electrode surface. In some variations, the electrode array may comprise a tissue contact layer. The tissue contact layer may function as a salt bridge between the electrodes and tissue. In some variations, the electrode array may comprise a hydrophilic coating. Additionally or alternatively, the electrode array may be electrically divided into sub-arrays to reduce drive current requirements. In some variations, the sub-arrays may correspond to the plurality of sections described herein.

In some variations, raised and/or rounded (e.g., semi-ellipsoid) electrodes may generally promote more reliable contact with tissue than flat electrodes and therefore a more uniform electrical field and improved treatment outcomes. For example, tissue contact (e.g., apposition) with the electrodes completes an electrical circuit during energy delivery and therefore provides the resistance in the circuit for a uniform electric field distribution. The raised and/or rounded (e.g., semi-ellipsoid) electrodes may reduce sharp edges to reduce arcing. The spaced-apart electrodes of the electrode array may further reduce ion concentration and associated electrolysis. The electrode array configurations (e.g., geometry, spacing, shape, size) shown and described herein provide uniform and spaced-apart electrodes that also allow a corresponding expandable member to repeatedly expand and compress.

In some variations, one or more of the electrodes (e.g., a plurality of the electrodes, a portion of the electrodes in an array, all of the electrodes in an array) may comprise one or more biocompatible metals such as gold, titanium, stainless steel, nitinol, palladium, silver, platinum, combinations thereof, and the like. In some variations, one or more electrodes (e.g., a plurality of the electrodes, a portion of the electrodes in an array, all of the electrodes in an array) may comprise an atraumatic (e.g., blunt, rounded) shape such that the electrode does not puncture tissue when pressed against tissue. For example, the electrode array may engage an inner circumference of the duodenum.

In some variations, the electrode array may be connected by one or more leads (e.g., conductive wire) to a signal generator. For example, a lead may extend through an elongate body (e.g., outer catheter, outer elongate body) to the electrode array. One or more portions of the lead may be insulated (e.g., PTFE, ePTFE, PET, polyolefin, parylene, FEP, silicone, nylon, PEEK, polyimide). The lead may be configured to sustain a predetermined voltage potential without dielectric breakdown of its corresponding insulation.

In some variations, an electrode array may comprise a plurality of elongate electrodes in a substantially parallel or interdigitated configuration. The shape and configuration of the electrode arrays described herein may generate an electric field of predetermined strength (e.g., between about 400 V/cm and about 7,500 V/cm) at a predetermined tissue depth (e.g., about 0.7 mm, about 1 mm) without excess heat, breakdown, steam generation, and the like. By contrast, some electrode configurations comprise a geometry (e.g., radius of curvature) where the electric fields generated decreases too quickly without application of very high voltages (e.g., thousands of volts) that may lead to the aforementioned excess heat, breakdown, and steam generation.

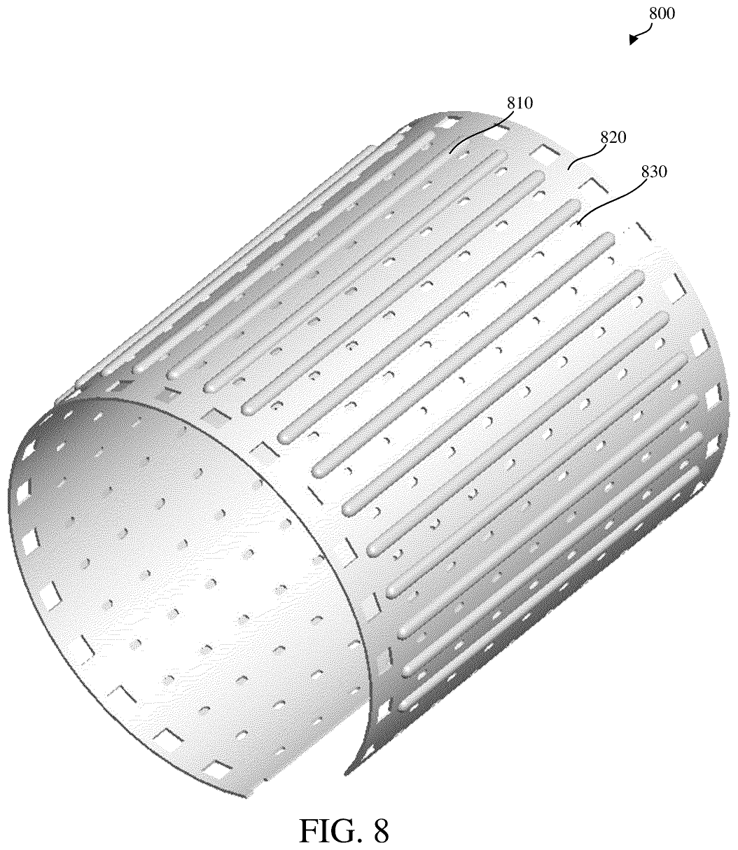

As described in detail herein, a pulsed electric field device may comprise an expandable member having a compressed (e.g., rolled) configuration and an expanded (e.g., unrolled) configuration. In some variations, the expandable member may comprise or may otherwise be formed from an electrode array (e.g., a plurality of electrodes). In some variations, the expandable member may comprise a flex circuit comprising a plurality of electrodes (e.g., electrode array). FIG. 8 is a perspective view of a variation of an electrode array (800) comprising a plurality of elongate electrodes (810) on a substrate (820) and a plurality of apertures (830). In some variations, the electrode array (800) may be in the form of a flex circuit. The flex circuit may comprise an electrode array (800) or a plurality of electrodes, for example, a plurality of elongate, parallel electrodes. In some variations, the substrate (820) of the electrode array (800) may define one or more apertures (830) (e.g., fluid openings) configured to generate suction (e.g., negative pressure) and/or output fluid (e.g., saline) between adjacent electrodes (810). The use of suction or negative pressure applied through the openings may draw tissue toward the electrode array (800) and may facilitate contact between the tissue and the electrode array (e.g., may increase a contact area between the surface of the tissue and the electrode surface). For example, the electrode array (800) may be engaged to the tissue via suction through the one or more apertures (830) that may promote more reliable (e.g., consistent) electrical contact between the pulsed electric field device and tissue, and therefore a more uniform electric field and an improvement to treatment outcomes. Furthermore, the applied suction may be configured to secure tissue apposition to the electrode array in a uniform manner. In some variations, a plurality of apertures (830) (e.g., row of openings) may be disposed between each pair of proximate (e.g., immediately adjacent) electrodes (810) with a predetermined spacing. For example, the apertures (830) may be spaced apart along a length of an electrode (820). In some variations, the aperture (830) may be disposed closer to one of the electrodes to promote contact between the tissue and at least one of the electrodes (810). Additionally or alternatively, the apertures (830) may be disposed equally between proximate electrodes (810) and/or through one or more electrodes (810).

In some variations, the electrode array may comprise a surface area between about 4 square centimeters and about 42 square centimeters, between about 6 square centimeters and about 10 square centimeters, between about 4 square centimeters and about 8 square centimeters, between about 20 square centimeters and about 42 square centimeters, between about 30 square centimeters and about 42 square centimeters, between about 10 square centimeters and about 30 square centimeters, and between about 8 square centimeters and about 42 square centimeters, including all ranges and sub-values in-between. In some variations, the expandable member in the unexpanded configuration may have an outer diameter between about 15 mm and about 20 mm, including all ranges and sub-values in-between.

Additionally or alternatively, the apertures (830) may be configured for fluid (e.g., gas, fluid) irrigation. The electrode array (800) may be in fluid communication with (e.g., fluidically coupled to) a fluid source (e.g., fluid source of saline, negative pressure source) for fluid irrigation and/or fluid cooling. For example, fluid may be removed from (e.g., suctioned out of) a body cavity or lumen after applying the pulsed or modulated electric field using the electrodes (810). In some variations, removal of the fluid may facilitate apposition and/or contact between the tissue and the electrode array (800).

In some variations, at least one of the electrodes (810) may comprise a semi-elliptical cross-sectional shape. In some instances, all of the electrodes (810) in the electrode array (800) may comprise a semi-elliptical cross-sectional shape. Generally, electric fields are intense near points and edges of electrodes due to the high concentration of surface charges there. Sharp-edged electrodes and high electric fields may generate one or more of electric discharge (e.g., arcing), high heat rates (e.g., boiling), high current density (e.g., electrolysis), and bubbles. The semi-elliptical cross-sectional shapes described herein may reduce one or more of these effects relative to sharp-edged electrodes. In some variations, a major axis of the electrode (810) is twice the electrode width and the minor axis of the electrode is equal to the electrode height in the middle of the electrode.

The electrode arrays described herein may be formed using any suitable manufacturing technique. The electrode arrays described herein may be manufactured using any suitable technique including, but not limited to, deposition of solder or other metal, dimpling of the substrate, plating of a metal (e.g., gold), and lamination. In some variations, additional layers and/or coatings may be applied to the electrode.

In some variations, a drive voltage applied to the electrode array may depend at least on the spacing between electrodes of the electrode array as well as electrode dimensions. For example, relatively wide elongate electrodes may reduce the effect of strong electric field intensities at sharply curved edges.

Additionally or alternatively, the plurality of elongate electrodes may comprise an interdigitated configuration. For example, the plurality of elongate electrodes may comprise a curved shape (e.g., S-shape, W-shape). The electrode array (810) may be configured to modify a flexural stiffness of the expandable member (800) to facilitate consistent expansion and compression of the expandable member (800). In some variations, the electrode array (810) may comprise a plurality of electrodes configured to protrude and/or recess relative to a surface of the substrate (820).

In some variations, a more uniform treatment of tissue (e.g., in areas where the electrode groups intersect) may be obtained by reducing the widths of the end-most electrodes of each section and reducing the distance between those electrodes. In some variations, a more uniform treatment of tissue (e.g., in areas where the electrode sections intersect) may be enabled by interdigitating the end-most electrodes of each group to overlap the treatment areas.

In some variations, an electrode array may comprise a plurality of electrode sections (e.g., zones), including 1, 2, 3, 4, 5, 6, 7, 8, 9, and 10 or more electrode sections. In some variations, each section of the plurality of sections may comprise a plurality of electrodes. For example, each section of the plurality of sections may comprises between 10 and 18 electrodes. In some variations, an electrode section of an electrode array may have a surface area of between about 250 mm² and about 1000 mm², between about 250 mm² and about 750 mm², between about 500 mm² and about 1000 mm², between 400 mm² and about 500 mm² and between 400 mm² and about 600 mm², including all ranges and sub-values in-between.

In some variations, the electrode array may be configured to generate a substantially uniform electric field at a predetermined tissue treatment depth across its entire surface. For example, a predetermined tissue depth may be configured to receive a voltage field of about 2,500 V/cm. A voltage of about 600 V with a current of about 50 A and a frequency of about 350 kHz may be applied at the electrodes. This may improve the consistency of energy delivery and treatment outcomes.

In some variations, a tissue treatment depth (e.g., 1 mm) receiving about a 2,500 V/cm voltage field may depend on an electrode configuration and the voltage applied to the electrode array. The current may depend on tissue conductivity and electrode configuration. Assuming a constant voltage, an electric field penetration is also constant. The tissue treatment ratio may depend on the state of the tissue during treatment (e.g., stretched, compressed, in-contact with the electrodes). The tissue treatment depth may depend on one or more of a tissue treatment ratio, current, effective voltage, and tissue type.

### Tissue Barrier

Generally, the tissue barriers described herein may be configured to protect the pulsed electric field device from catching (e.g., sticking, holding) against tissue that may otherwise restrict movement and/or operation of the pulsed electric field device. For example, the tissue barrier may reduce the likelihood of tissue becoming trapped with respect to one or more of an expandable member, first elongate body, and second elongate body of the pulsed electric field device during one or more steps of a treatment procedure, such as when suction is applied through a lumen of the expandable member. The positioning and geometry of the tissue barrier may deflect, separate, or ‘wipe off’ tissue from portions of the device prone to tissue catching. In some variations, the tissue barrier may be coupled between an edge of the expandable member and a corresponding portion of the elongate body.

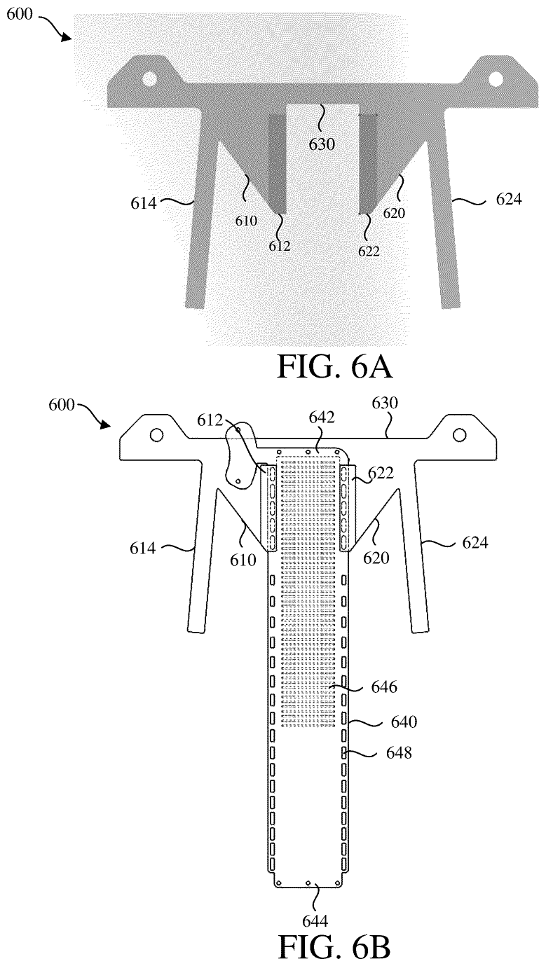

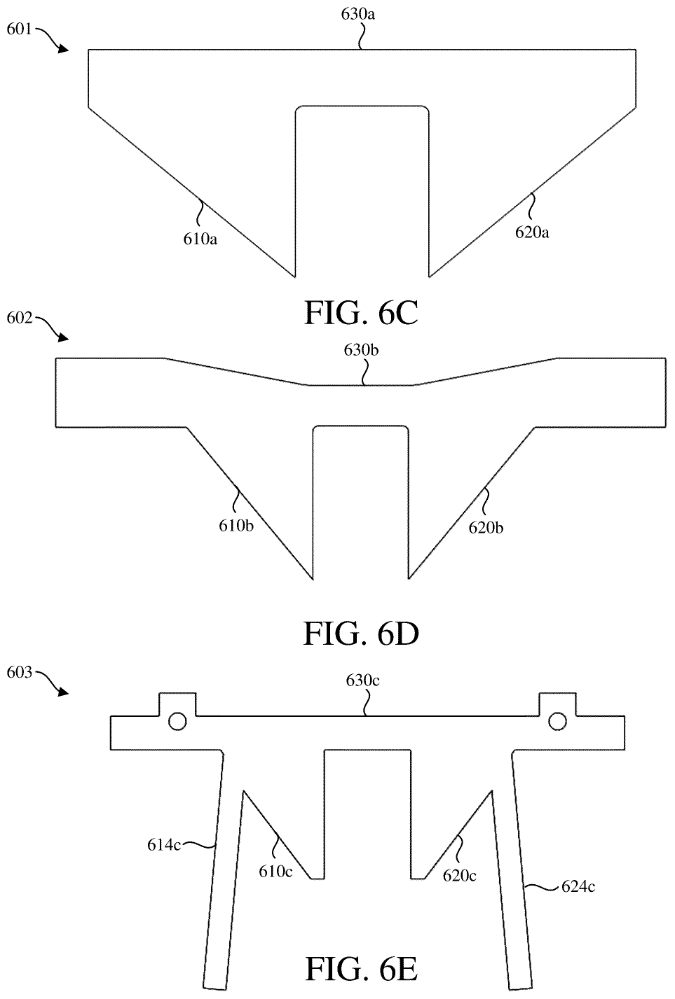

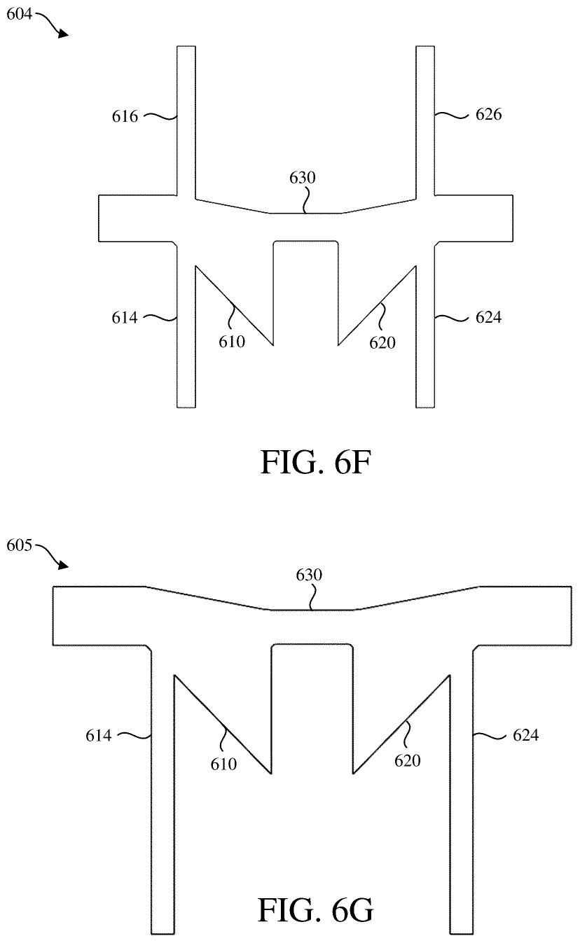

FIGS. 6A-6G are schematic diagrams of variations of a tissue barrier (600-605) of a pulsed electric field device. FIG. 6A depicts a tissue barrier (600) comprising a first portion (610), a second portion (620), and a third portion (630) coupled therebetween. In some variations, the first portion (610) may comprise a first expandable member coupling portion (612) and a first dilator coupling portion (614). In some variations, the second portion (620) may comprise a second expandable member coupling portion (622) and a second dilator coupling portion (624).

In some variations, the first and second expandable member coupling portions (612, 622) may be configured to couple to respective lengthwise edges of an expandable member (640). For example, FIG. 6B depicts the tissue barrier (600) of FIG. 6A coupled to an expandable member (640). In some variations, the first and second expandable member coupling portions (612, 622) may be heat sealed through the tracks (648) of the expandable member (640). The expandable member (640) may be similar to the expandable member (520) of FIGS. 5A-5R and may comprise an outer end (642), an inner end (644), an electrode array (646), and a pair of tracks (648). Each of the electrode array (646) and the tracks (648) may be configured to extend from the outer end (642) to the inner end (644). In some variations, the first and second dilator coupling portions (614, 624) may be angled with respect to a longitudinal axis (e.g., extending horizontally along FIG. 6A) of the tissue barrier (600).

In some variations, the first and second dilator coupling portions (614, 624) may be configured to couple to a respective first and second dilator (e.g., first and second portions (512, 514)) of an elongate body (e.g., first elongate body (510). For example, the first and second dilator coupling portions (614, 624) may be configured to wrap around the first and second dilators to attach the tissue barrier (600) to the first elongate body (not shown for the sake of clarity). In some variations, the first and second dilator coupling portions (614, 624) may be configured to wrap around at least an entire circumference of a respective first and second dilator. The first and second dilator coupling portions (614, 624) may have a generally rectangular shape that forms linear strips that may be wound around a respective dilator. In some variations, the first and second dilator coupling portions (614, 624) may comprise an adhesive such as a UV adhesive for bonding to the first and second dilators.

In some variations, the first and second portions (610, 620) (e.g., wings) of the tissue barrier (600) in the unrolled configuration may form a pair of right triangles. Each triangle of the pair of right triangles may comprise an acute angle of between about 20 degrees and about 90 degrees. In some variations, the first portion (610) and the second portion (620) may be bilaterally symmetric. For example, a shape of the first portion (610) and the second portion (620) may be the same. In some variations, the tissue barrier does not enter into an opening (512) of the elongate body (510). In some variations, the tissue barrier 600 does not overlap with a cover (550).

The material, properties, and dimensions of the tissue barrier may ensure that the tissue barrier is strong enough to deflect tissue from catching against one or more portion of the pulsed electric field device while also having sufficient flexibility to be reproducibly roll and unroll without becoming entangled with itself or tissue. In some variations, the tissue barrier may comprise one or more of a thermoplastic polyurethane (TPU), a ultra-high molecular weight polyethylene (UHMWPE), a polyether block amide, a polyimide, and combinations thereof. In some variations, the tissue barrier may be transparent to aid visualization of the treatment procedure. In some variations, the tissue barrier may comprise a durometer between about 10 Shore A and about 100 Shore A, between about 10 Shore A and about 50 Shore A, between about 50 Shore A and about 100 Shore A, between about 25 Shore A and about 75 Shore A, including all ranges and sub-values in-between.

In some variations, a distance between the first portion (610) and the second portion (620) may be between about 3 mm and about 20 mm, between about 3 mm and about 10 mm, between about 10 mm and about 20 mm, between about 5 mm and about 10, and between about 3 mm and about 5 mm, including all ranges and sub-values in-between. In some variations, the tissue barrier may comprise a thickness of between about 0.125 mm and about 0.8 mm, between about 0.125 mm and about 0.5 mm, and between about 0.5 mm and about 0.8 mm, including all ranges and sub-values in-between. In some variations, the tissue barrier may couple to each of the proximal edge and the distal edge for a length of between about 25 mm and about 130 mm, between about 25 mm and about 100 mm, between about 50 mm and about 100 mm, between about 100 mm and about 130 mm, between about 25 mm and about 50 mm, and between about 25 mm and about 75 mm, including all ranges and sub-values in-between.

FIG. 6C is a schematic diagram of a tissue barrier (601) comprising a first portion (610a), a second portion (620a), and a third portion (630a) positioned therebetween. The first and second portions (610a, 620a) of the tissue barrier (601a) in the unrolled configuration form a pair of right triangles (610a, 620a) having a surface area of the tissue barrier (601) of up to about 90%, of up to about 80%, of up to about 70%, of up to about 60%, including all ranges and sub-values in-between. Furthermore, the triangles are configured to directly roll and unroll about the first and second dilators, respectively. FIG. 6D is a schematic diagram of a tissue barrier (602) comprising a first portion (610b), a second portion (620b), and a third portion (630b) positioned therebetween. The third portion (630b) has a tapered shape, and the first and second portions (610b, 620b) of the tissue barrier (602) in the unrolled configuration form a pair of right triangles having a larger surface area than that of the tissue barrier of FIG. 6A (600). The tissue barriers described herein need not include a dilator coupling portion which are not included in, for example, tissue barriers (601, 602) of FIGS. 6C and 6D.

FIG. 6E is a schematic diagram of a tissue barrier (603) similar to the tissue barrier (601) of FIG. 6C and comprising a first portion (610c), a second portion (620c), and a third portion (630c) positioned therebetween. Similarly to FIG. 6C, the triangles (610c, 620c) of FIG. 6E are configured to directly roll and unroll about the first and second dilators, respectively. The triangles (610c, 620c) are further configured to couple to the first and second dilators via first and second dilator coupling portions (614c, 624c). FIG. 6F is a schematic diagram of a tissue barrier (604) comprising a first portion (610), a second portion (620), and a third portion (630) positioned therebetween. The third portion (630) has a tapered shape beginning from about an inner edge of the triangles (610b, 620b) to aid manufacturability. The first and second dilator coupling portions (614, 624) may be perpendicular with respect to a longitudinal axis (e.g., extending horizontally along FIG. 6F) of the tissue barrier (604). Furthermore, the first portion (610) further comprises a third dilator coupling portion (616), and the second portion (620) further comprises a fourth dilator coupling portion (626).

FIG. 6G is a schematic diagram of a tissue barrier (605) comprising a first portion (610), a second portion (620), and a third portion (630) positioned therebetween. The third portion (630) has a tapered shape, and the first and second dilator coupling portions (614, 624) may be perpendicular with respect to a longitudinal axis (e.g., extending horizontally along FIG. 6G) of the tissue barrier (605). Furthermore, the first and second dilator coupling portions (614, 624) may have a longer length relative to the corresponding dilator coupling portions of the tissue barrier (600). For example, a ratio of a dilator portion length to a triangle length (e.g., expandable member coupling portion length) may be between 3:1 to 1:1, between 2:1 to 1:1, and between 1.5:1 to 1:1, including all ranges and sub-values in-between. The tissue barriers (600-605) may be formed as a single-piece construction or in a plurality of pieces and separately coupled. In some variations, as shown in FIG. 6A, the third portion (630) may extend longitudinally beyond the ends of the first portion (610) and second portion (620) to aid one or more of coupling to the device, production, and manufacturability. The dilator coupling portions (614, 625) may extend from the triangles (610, 620).

As best understood with respect to FIGS. 5A, 5C, 5E, 5G, 5H, and 5M, in some variations, the first and second portions of the tissue barrier (540a, 540b) may be rolled about a longitudinal axis of the first elongate body (510). For example, the tissue barrier (540a, 540b) in the rolled configuration may comprise a plurality of turns about the first elongate body (510). The tissue barrier (540a, 540b) may be configured to transition between a rolled configuration and an unrolled configuration. For example, FIGS. 5B, 5D, 5F, 5I, 5J, 5K, 5N, 5O, 5P, and 5R depict the tissue barrier (540a, 540b) in an unrolled configuration. In some variations, the second elongate body (560) may be configured to rotate relative to the first elongate body (510) to transition the tissue barrier (540a, 540b) between a rolled configuration and an unrolled configuration.

The tissue barrier in the expanded configuration may have a three-dimensional shape as shown in the first and second portions (540a, 540b) of FIGS. 5F, 5J, 5N, and 5O where the triangles (610, 620) are unrolled. For example, the first and second portions (540a, 540b) form a partial tent or open conical-like shape that protects a lumen (521) and the expandable member (520) from tissue. The dilator coupling portions (614, 624) remain coupled to respective dilators to maintain attachment of the tissue barrier to the dilators. In the unexpanded configuration, the first and second portions (540a, 540b) roll around an outer surface of a dilator to generally conform to the shape of their respective dilators (512, 514). An edge of the first and second portions (540a, 540b) may form a curved surface as shown in, for example, FIG. 5H where the first and second portions (540a, 540b) may not roll completely around the dilators such that a portion of the dilators (512, 514) may be exposed in the unexpanded configuration.

In some variations, the tissue barrier may comprise a tensile strength of up to about 5 lb, between about 3 lb and about 5 lb, and between about 3 lb and about 4 lb, including all ranges and sub-values in-between. In some variations, the tissue barrier in an unrolled configuration may withstand a force of up to about 14 ft/lb, between about 5 ft/lb and about 14 ft/lb, and between about 10 ft/lb and about 14 ft/lb, including all ranges and sub-values in-between.

### Cover

Generally, the covers described here may be configured to protect the pulsed electric field device from catching (e.g., sticking, holding) against tissue so as to restrict movement and/or operation of the pulsed electric field device. For example, the cover may prevent tissue from being caught between the expandable member and an opening of the first elongate body, as well as caught in the opening of the first elongate body.

The positioning and geometry of the cover may be configured to reduce a size of an opening of the first elongate body for tissue to enter and may further be configured to ‘wipe off’ or deflect tissue drawn towards the opening of the first elongate body from portions of the device prone to tissue catching, thereby facilitating transition of the expandable member between rolled and unrolled configurations. For example, the cover may be configured to overlap a portion (e.g., opening) of the first elongate body where the expandable member projects outward from, thus preventing tissue from catching between the first elongate body and expandable member when transitioning between rolled and unrolled configurations.

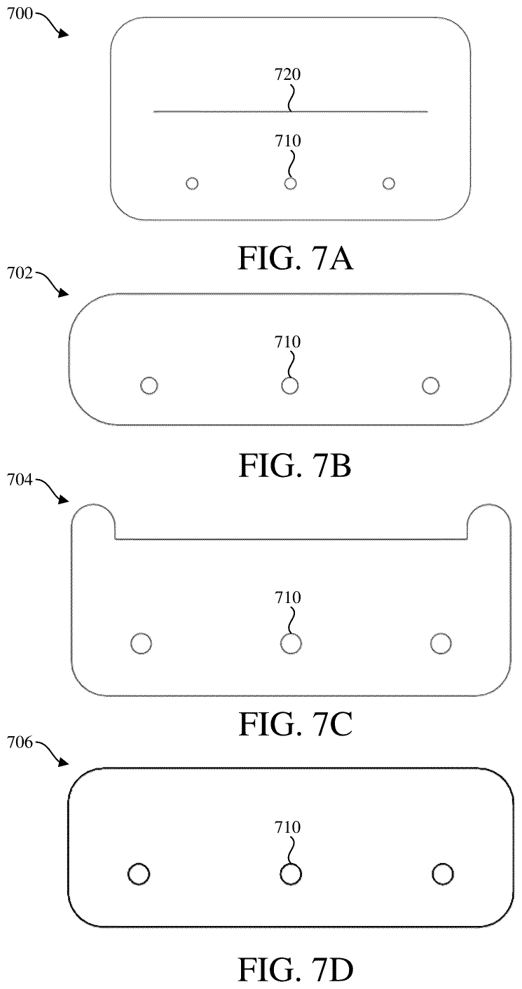

FIGS. 7A-7D are schematic views of variations of a cover (700-706) of a pulsed electric field device. In some variations, the cover (700-706) may have an atraumatic shape so as to facilitate atraumatic separation of tissue (e.g., tissue wiping) from the pulsed electric field device that minimizes tissue damage (e.g., perforation). FIG. 7A depicts a cover (700) comprising a hole connector (710) and a slit (720). The slit (720) may be configured to receive an expandable member (e.g., expandable member (520)) therethrough. In some variations, the slit (720) may be parallel to a longitudinal axis of the first elongate body. Rolling and unrolling the expandable member may translate the expandable member through the slit (720).

In some variations, the hole connector (710) of the cover (700-706) may comprise a plurality of holes (e.g., openings, apertures) configured to receive a connector (e.g., first connector (513)) for coupling to a first elongate body (e.g., first elongate body (510)). However, the cover (700-706) need not comprise a hole connector (710) and may be coupled to a pulsed electric device in any suitable manner.

The spatial relationship between the cover, first elongate body, and expandable member in the unexpanded and expanded configurations is shown in FIGS. 5Q and 5R. With respect to FIG. 5Q, the outer end (522) of the expandable member (520) may be coupled to the first elongate body (510) through a first connector (513). In some variations, the cover (550) may be coupled to one or more of the expandable member (520) and the first elongate body (510) through the first connector (513). For example, the cover (550) may be coupled between the expandable member (520) and the first elongate body (510) and/or the expandable member (520) may be coupled between the cover (550) and the first elongate body (510). Optionally, one or more leads may be coupled to the electrode array (530) through the first elongate body (510) and the first connector (513).

In some variations, the cover (550) may overlap a portion of the expandable member (520) as the expandable member (520) moves relative to (e.g., rolls) about the first elongate body (510). In some variations, the cover (550) may extend across (e.g., overlap, cover) an entire opening (512) of the first elongate body (510). For example, the cover (550) may extend across the entire opening (512) of the first elongate body (510) through which the expandable member (520) is positioned to facilitate expanding the expandable member (520).

The cover may have an atraumatic curve to reduce injury to tissue, follow a curve of the first elongate body, and to apply a downward force (e.g., sandwich) to the expandable member between the cover and the first elongate body. In some variations, the cover (550) may comprise a concave shape having a radius of curvature between about 6 mm and about 16 mm, between about 6 mm and about 10 mm, between about 10 mm and about 16 mm, and between about 8 mm and about 12 mm, including all ranges and sub-values in-between. In some variations, the cover (550) may comprise a width of between about 23 mm and about 38 mm, between about 23 mm and about 30 mm, between about 30 mm and about 38 mm, and between about 25 mm and about 35 mm, including all ranges and sub-values in-between. As shown in FIG. 5G, for example, a length of the cover (550) may be greater than or equal to a width of the expandable member (520). In some variations, a length of the cover (550) may be between about 10 mm and about 50 mm, between about 10 mm and about 40 mm, between about 10 mm and about 30 mm, and between about 15 mm and about 25 mm, including all ranges and sub-values in-between. In some variations, the cover (550) may comprise one or more atraumatic edges. For example, the cover (550) may comprise radiused corners. In some variations, the cover (550) may be configured to reduce contact between tissue and the second elongate body (560). In some variations, the cover (550) may comprise a hardness of between about 40D and about 75D, including all ranges and sub-values in-between.

In some variations, the cover (550) may sandwich a portion of the expandable member (520) between the cover (550) and the first elongate body (510). For example, as shown in FIG. 5Q, the cover may comprise a first portion (550a) facing an outer side of the expandable member (520) and a second portion (550b) facing an inner side of the expandable member. In some variations, the second portion (550b) may be coupled to the first elongate body (510).

In some variations, the cover may comprise one or more of a thermoplastic polyurethane (TPU), a ultra-high molecular weight polyethylene (UHMWPE), a polyether block amide, a polyimide, and combinations thereof. The cover may optionally have a different color than the expandable member so as to aid orientation of the pulsed electric field device relative to tissue.

### Actuator

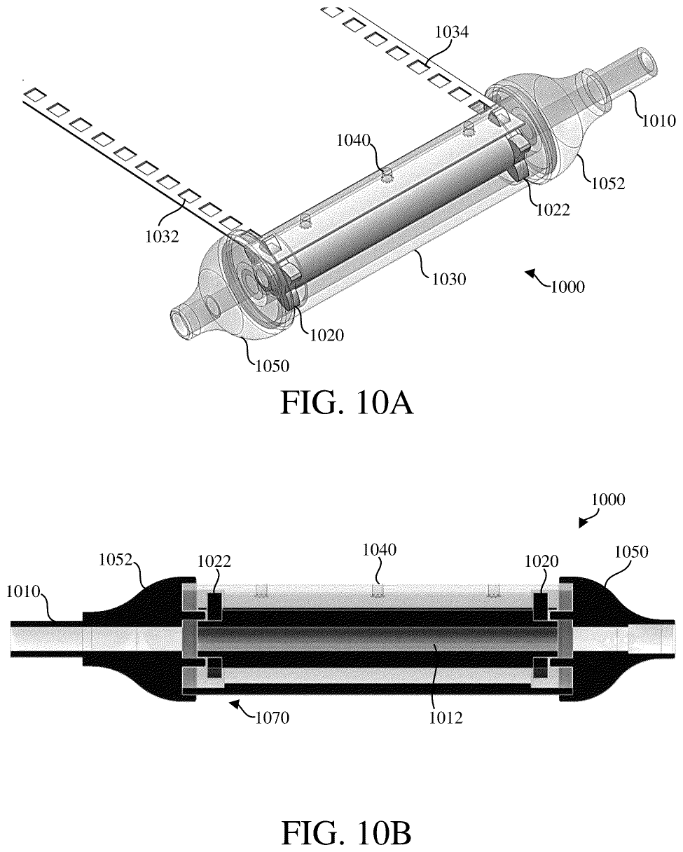

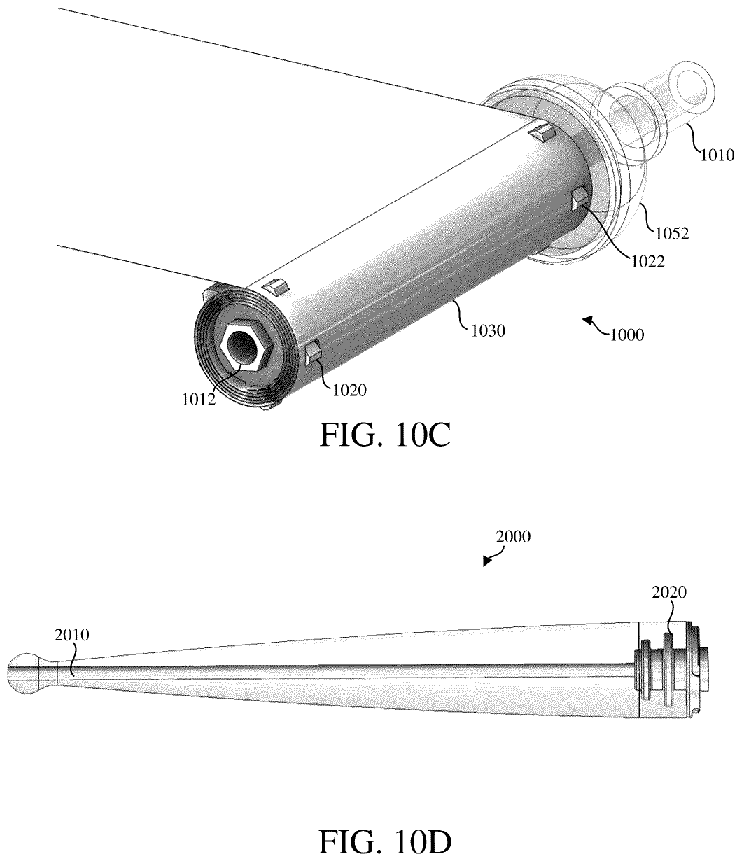

In some variations, an expandable member of a pulsed electric field device may transition configurations by using an actuator that allows improved control over the expansion and/or compression of the expandable member. For example, in variations in which a rolled expandable member is used, the actuator may comprise a set of gears and/or friction rollers (e.g., knurled friction rollers), and tracks configured for consistent transmission of rotational torque from the rotating elongate body to the expandable member. FIG. 10A is a perspective view and FIG. 10B is a cross-sectional side view of a variation of a pulsed electric field device (1000) comprising an actuator (1070). As shown there, the pulsed electric field device (1000) may comprise a first elongate body (1010) comprising a lumen therethrough and a second elongate body (1012) at least partially positioned within the lumen of the first elongate body (1010), and one or more actuators (1070). The pulsed electric field device (1000) may further comprise an expandable member (1030) rolled about the second elongate body (1012), as described in more detail herein, and operably coupled to the actuator (1070). In some variations, the pulsed electric field device (1000) may further comprise one or more dilators, for example, a distal dilator (1050) (e.g., corresponding to a distal portion (512)) and a proximal dilator (1052) (e.g., corresponding to a proximal portion (514)), coupled to one of the first elongate body (1010) and the second elongate body (1012). In some variations, one or more of the dilators (1050, 1052) may have a sigmoidal shape. The actuator (1070) may be disposed between the distal dilator (1050) and the proximal dilator (1052). The expandable member (1030) may be disposed between the distal dilator (1050) and the proximal dilator (1052).

As mentioned above, the pulsed electric field device (1000) may comprise an actuator operably coupled to the expandable member (1030) and configured to assist in expanding (e.g., unrolling) and compressing (e.g., rolling) the expandable member (1030). In some variations, the actuator may comprise one or more gears, which may interface with the expandable member (1030), such as, for example, via one or more tracks formed in the expandable member (1030). For example, in the variation depicted in FIGS. 10A-10C, the actuator (1070) may comprise a first gear (1020) and a second gear (1022), each of which may be coupled to the second elongate body (1012). The expandable member (1030) may further comprise a first track (1032) on a first side thereof and a second track (1034) on a second side thereof. The first track (1032) may be operably coupled to the first gear (1020) and the second track (1034) may be operably coupled to the second gear (1022). In some of these variations, the first and/or second tracks (1032, 1034) may comprise a plurality of spaced apart openings in the expandable member (1030) configured to receive the teeth of the respective gears (1020, 1022). The expandable member (1030) may be coupled to the second elongate body (1012) via the gears (1020, 1022). FIG. 10C is a detailed cutaway perspective view of the pulsed electric field device (1000) depicting engagement of the teeth of the gears (1020, 1022) with the respective tracks (1032, 1034) of the expandable member (1030). Additionally or alternatively, the actuator may comprise a metal roller comprising a plurality of teeth textures configured to directly press against the expandable member (1030). The metal roller may be configured to operate with a drum plotter or a film canister type of mechanism. Similar to the pulsed electric field device (500) of FIGS. 5A-5R, the expandable member (1030) may comprise an inner end (e.g., innermost portion of roll) and an outer end (e.g., outermost portion of roll) where the inner end is coupled to the second elongate body (1012) and the outer end is coupled to the first elongate body (1010). A direction of the rotation (e.g., clockwise, counter-clockwise) of the second elongate body (1012) may determine the expansion or compression of the expandable member (1030). In some variations, a connector (1040) may couple the second elongate body (1012) to the inner end of the expandable member (1030). An outer end of the expandable member (1030) may be coupled to one or more of the dilators (1020, 1022) and the first elongate body (1010). However, FIG. 10A shows an unattached outer end of the expandable member (1030) for the sake of illustration. In some variations, the expandable member (1030) in the rolled configuration may have a diameter between about 6 mm and about 15 mm, including all ranges and sub-values in-between. The expandable member (1030) in the rolled configuration may comprise one or more turns. In some variations, the expandable member (1030) in the expanded configuration may have a diameter between about 10 mm and about 50 mm, including all ranges and sub-values in-between.

In some variations, the electrode array may be electrically coupled to the second elongate body (1012) through the connector (1040). For example, one or more leads may couple to the electrode array through the second elongate body (1012) and connector (1040). Additionally or alternatively, one or more leads may couple to the electrode array through the first elongate body (1010). In some variations, a cover such as cover 700-706 (not shown for the sake of clarity) may be coupled to one or more of the expandable member (1030) and the first elongate body (1010) through the connector (1040). For example, the cover may be coupled between the expandable member (1030) and the first elongate body (1010) and/or the expandable member (1030) may be coupled between the cover and the first elongate body (1010).

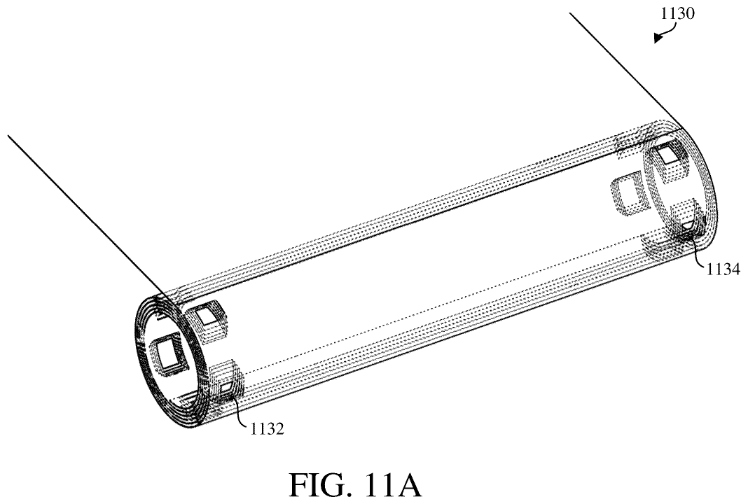

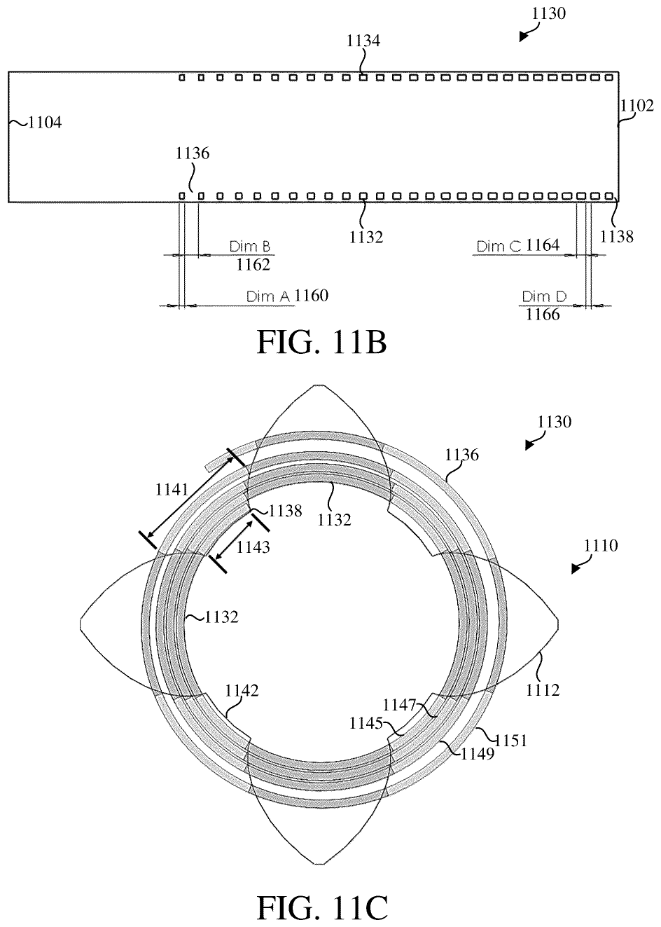

FIG. 11A is a perspective view of a variation of an expandable member (1130) of the pulsed electric field device (1100) depicting the expandable member (1130) in the compressed configuration and corresponding alignment of the openings of the tracks (1132, 1134). The openings of the tracks (1132, 1134) may be sized and positioned to substantially overlap with each other when the expandable member (1130) is in the compressed configuration such that the teeth of the gears (e.g., gears (1020, 1022)) may pass through and be positioned within a plurality of the openings in a track (1132, 1134), as will be described in more detail herein. In some variations, the size and spacing of the tracks (1132, 1134) may change along a length of the expandable member (1130) to aid smooth rolling and unrolling.

FIG. 11B is a plan view of the expandable member (1130) and the tracks (1132, 1134) in an unrolled configuration. In some variations, a distance between adjacent openings (e.g., tracks) (1162, 1166) may change along a length of the expandable member (1130). In particular, a distance (1162, 1166) between adjacent openings may increase along a longitudinal axis of the expandable member (1130) from a first end (1102) of the expandable member to a second end (1104) of the expandable member. For example, Dim D (1166) adjacent to or near the first end (1102), or in a first portion of the expandable member (1130) at the first end (first end portion), may be smaller than Dim B (1162) adjacent to or near the second end (1104), or in a second portion of the expandable member (1130) at the second end (second end portion). Conversely, a length of each opening (1160, 1164) may decrease along a longitudinal axis of the expandable member (1130) from the first end (1102) to the second end (1104). For example, a length of Dim C (1164) adjacent to or near the first end (1102) or in the first end portion may be greater than a length of Dim A (1160) adjacent to or near the second end (1104) or in the second end portion. This spacing and opening geometry may allow the expandable member to form a more precise and compact shape about a gear in the rolled configuration, as shown in FIG. 11C described in more detail below.

An expandable member (1130) comprising variable length openings and distances between openings may allow for a more compact rolled configuration around a gear comprising a gear body (1142) and curved or angled teeth extending therefrom, as shown FIG. 11C. FIG. 11C is an illustrative variation of an expandable member (1130) (such as the expandable member shown in FIG. 11B) in a rolled configuration. The expandable member (1130) is depicted rolled around a gear (1110) comprising one or more teeth (1112). While depicted in FIG. 11C as a cylindrical gear (e.g., having a cylindrical body), the gear (1110) need not be and the gear body (1142) may have any suitable cross-sectional shape, such as, for example, elliptical, square, rectangular, and the like. Each tooth (1112) may comprise a predetermined tapered (e.g., sloped, curved) shape configured to facilitate equal load transfer between openings of the tracks (1132, 1134). The variable spacing and opening geometry of the expandable member (1130) may facilitate precise rolling of the expandable member about the gear (1110). In the rolled configuration shown in FIG. 11C, the expandable member (1130) may comprise one or more overlapping layers (e.g., turns). For example, in a radial outward direction from a radial center of the rolled expandable member (1130), the expandable member (1130) may comprise a first layer (1145) (inner most layer), a second layer (1147), third layer (1149), and a fourth layer (1151) (outer most layer). A number of layers of the expandable member (1130) in a rolled configuration may be based at least on a length and thickness of the expandable member, a diameter of a gear, a number of teeth, and the like. A distance (1141, 1143) (e.g., spiral pitch) between adjacent openings (e.g., tracks) may increase from the first layer (1145) to the fourth layer (1151) (e.g., in a radial outward direction). A length (1141) of an opening (1132) may decrease from the first layer (1145) to the fourth layer (1151) (e.g., in a radial outward direction). This may allow the expandable member (1130) to be rolled around the gear (1110) with minimal spacing between layers. Therefore, the openings the tracks (1132, 1134) may fit smoothly onto and/or around the gear teeth (1112), while the portions of the expandable member (1130) between the tracks (1132, 1134) may fit smoothly around the gear body between the gear teeth (1112), which may reduce interference, binding, and bunching of the expandable member (1130) in the rolled configuration.

In some variations, the expandable member (1130) (e.g., circuit substrate, flex circuit) may comprise an electrode array (not shown for the sake of clarity) which may comprise any of the electrode arrays described herein. For example, the electrode array may be disposed on an outer surface of the expandable member (1130).

In some variations, a distance (1141, 1143) (e.g., spiral pitch) between the openings of the tracks (1132, 1134) may be a function of a thickness of the expandable member (1130) and the number of turns (e.g., layers) of the expandable member (1130). For example, the expandable member (1130) may comprise one or more electrodes (e.g., electrode pad) of an electrode array (not shown in FIG. 11A-11C) that may increase a thickness of those portions of the expandable member (1130). The length of an opening (1132, 1134) and/or distance between adjacent openings may increase with increasing thickness of the expandable member (1130).

In some variations, the second elongate body (1112) (e.g., inner torsion member, rotatable member) may be configured to rotate relative to the first elongate body (1110) to transition the expandable member (1130) between the rolled configuration and the unrolled configuration. In some of these variations, the expandable member (1130) may comprise a lumen of at least 10 mm in diameter in the unrolled configuration.

### Elongate Body

Generally, the elongate bodies (e.g., catheters) of the pulsed electric field devices described herein may be configured to deliver an electrode array to a target tissue for treating the tissue. In some variations, an elongate body may comprise a shaft composed of a flexible polymeric material such as Teflon, Nylon, Pebax, urethane, combinations thereof, and the like. In some variations, the pulsed electric field device may comprise one or more steerable or deflectable catheters (e.g., unidirectional, bidirectional, 4-way, omnidirectional). In some variations, the elongate body may comprise one or more pull wires configured to steer or deflect a portion of the elongate body. In some variations, the elongate body may have a bend radius between about 5 cm and about 23 cm and/or between about 45 degrees and about 270 degrees. In some variations, the elongate bodies described herein may comprise a lumen through which another elongate body and/or a guidewire may slide. In some variations, the elongate bodies may comprise a plurality of lumens. For example, the elongate body may comprise one or more of an inflation lumen, fluid lumen, guidewire lumen, and lead lumen.

In some variations, a first elongate body may have a length of between about 150 cm and about 200 cm, between about 170 cm and about 200 cm, between about 180 cm and about 190 cm, and between about 150 cm and about 170 cm, including all ranges and sub-values in-between. In some variations, a first elongate body may decrease in stiffness proximally to facilitate navigation of the pulsed electric field device through one or more body cavities or lumens. For example, a distal portion of the first elongate body may comprise a stiffness of between about 45D and about 70D, between about 50D and about 60D, and about 55D, including all ranges and sub-values in-between. The distal portion of the first elongate body may have a length of between about 10 inches and about 30 inches, between about 15 inches and about 25 inches, between about 15 inches and about 20 inches, and about 17 inches, including all ranges and sub-values in-between. A proximal portion of the first elongate body may comprise a stiffness of between about 50D and about 100D, between about 60D and about 80D, between about 65D and about 75D, and about 70D including all ranges and sub-values in-between. The proximal portion of the first elongate body may have a length of between about 40 inches and about 70 inches, between about 50 inches and about 60 inches, and about 55 inches, including all ranges and sub-values in-between.

In some variations, a first elongate body may have a diameter of between about 1 mm and about 20 mm, between about 5 mm and about 15 mm, between about 5 mm and about 10 mm, and between about 10 mm and about 20 mm, including all ranges and sub-values in-between.

In some variations, a lumen of a first elongate body may have a diameter of up to about 2 mm, up to about 1.5 mm, up to about 1 mm, up to about 0.5 mm, between about 1 mm and about 2 mm, and between about 1 mm and about 1.5 mm, including all ranges and sub-values in-between.

In some variations, the elongate body may be woven and/or braided and/or coiled, and may be composed of a material (e.g., nylon, stainless steel, nitinol, polymer) configured to enhance pushability, torquability and flexibility. In some variations, one or more of the first and second elongate bodies may comprise a metal-based radiopaque marker comprising one or more of a ring, band, and ink (e.g. platinum, platinum-iridium, gold, nitinol, palladium) configured to permit fluoroscopic visualization. In some variations, one or more of the first and second elongate bodies may comprise magnetic members configured to attract and couple to the bodies to each other. In this manner, the first elongate body need not comprise a lumen for the second elongate body. In some variations, the elongate body may comprise from about 2 layers to about 15 layers of materials to achieve a predetermined set of characteristics.

In some variations, the first elongate body and visualization device may be coupled along a predetermined length using one or more of a coupling sleeve, a plurality of rings, and mechanical fasteners. For example, the coupling sleeve may comprise one or more of a polymer sleeve having a spine optionally including scalloped edges, a tubular braid (e.g., Nylon, PET), a balloon polymer sleeve (e.g., baleeve), and EPTFE biaxially oriented. The plurality of rings may include a chain of rings that may be FEP coated and/or formed of silicone and/or Viton.

### Dilator

Generally, the dilators of the pulsed electric field devices described here may be configured to assist advancement of one or more portions of a pulsed electric field device into and through a body cavity or lumen. In some variations, a dilator may generally be configured to dilate a body cavity or lumen, such as a lumen of a duodenum. The dilator may be atraumatic in shape to minimize any inadvertent or unintended damage and may comprise any shape suitable to enlarge a tissue lumen. For example, in some variations, a dilator may comprise a conical shape comprising a taper of between about 1 degree and about 45 degrees, which may facilitate PEF device advancement through a body lumen, such as a portion of the gastrointestinal tract. In some variations, the dilator may comprise PET, PEBA, PEEK, PTFE, silicone, elastomer, PS, PEI, latex, sulphate, barium sulfate, a copolymer, combinations thereof, and the like. In some variations, the dilator may comprise a solid configuration. In some variations, the dilator may comprise a plurality of materials configured to provide a desired stiffness and compliance along a length of the dilator. The dilator may comprise one or more components configured to facilitate advancement of a guidewire.

In some variations, the dilator may comprise a length of between about 2 mm and about 10 cm. In some variations, the dilator may comprise a taper of between about 5 degrees and about 30 degrees relative to a longitudinal axis of the dilator. Furthermore, a distal end of the dilator may be atraumatic (e.g., rounded, blunted). In some variations, a pulsed electric field device may comprise a plurality of dilators (e.g., 2, 3, 4, 5, 6, or more). For example, respective dilators may be disposed proximal and distal to an expandable member. This allows smooth proximal and distal advancement of the pulsed electric field device. In some variations, the dilator may comprise a shore A hardness of between about 30 Shore A and about 50 Shore A, and between about 40 Shore A and about 50 Shore A, including all ranges and sub-values in-between.

In some variations, a pulsed electric field device may comprise one or more dilators configured to aid advancement of the device through one or more tortuous body cavities without damaging tissue. FIG. 10A is a perspective view of a variation of a pulsed electric field device (1000) in a rolled configuration where the pulsed electric field device (1000) comprises one or more dilators (1010, 1050). For example, the pulsed electric field device (1000) may comprise a distal dilator (1050) and a proximal dilator (1052), each coupled to one of the first elongate body (1010) and the second elongate body (1020). The dilators (1050, 1052) may assist in smoothly advancing and/or retracting the pulsed electric field device (1000) through one or more body cavities or lumens and may assist in preventing the expandable member from catching on tissue. For example, the dilators (1050, 1052) may be configured to protect an edge of the expandable member (1030) and second elongate body (e.g., canister) from contacting tissue as it is being advanced through a body cavity. In some variations, an outer surface at the end of the actuator adjacent the expandable member may be level with (e.g., at the same height) as the expandable member in the expanded configuration, be lower than or be higher than the expandable member. Accordingly, the dilators (1050, 1052) may allow the pulsed electric field device (1000) to be smoothly translated through one or more body cavities, as described in more detail herein.

The expandable member (1030) may be disposed between the distal dilator (1050) and the proximal dilator (1052). The length and taper of the dilators of the device may be the same or different. For example, a distal dilator (1050) may have a steeper taper than the proximal dilator (1052) and vice versa. In some variations, the distal dilator may have a longer length than a proximal dilator and vice versa. In some variations, the pulsed electric field device may comprise just a single distal dilator.

In some variations, a dilator may comprise a recess configured to facilitate mating or coupling with another elongate member such as a visualization device (e.g., endoscope). For example, this may enable the dilator and expandable member to removably couple to a visualization device during a treatment procedure.

FIG. 10D depicts a variation of a dilator (2000) including a lumen (2010) and one or more flanges (2020) configured to provide variable stiffness along a length of the dilator (2000) to facilitate translation through a tortuous body cavity or lumen. For example, a conventional dilator having insufficient stiffness may deflect and buckle when navigating through a curvature (e.g., around a corner) while a dilator having too much stiffness may traumatically engage with tissue during navigation through a body cavity. By contrast, the increasing stiffness provided distally along a length of the dilator (2000) enables a proximal end of the dilator (2000) to deflect more easily than a distal end while the distal end of the dilator (2000) comprising the flanges (2020) provides stiffness such that the dilator (2000) does not buckle or deform. In some variations, the stiffness may increase from a distal end of the dilator to a proximal end of the dilator by up to 10%, by about to about 20%, by about to about 30%, by about to about 40%, by about to about 50%, including all ranges and sub-values in-between. In some variations, the stiffness of the dilator may increase linearly or non-linearly along a length of the dilator. The dilator (2000) may further comprise an atraumatic enlarged bulbous distal tip. The lumen (2010) may be configured to receive one or more of a guidewire, visualization device, and the like. The flanges (2020) may comprise one or more rings disposed along and perpendicular to a longitudinal axis of the dilator (2000). The dilator (2000) may comprise additional lumens. Alternatively, the dilator (2000) may not include a lumen. In some variations, the first dilator (200) may comprise one or more fiducial markers (e.g., radiopaque markers). For example, the fiducial marker may comprise a metal-based radiopaque marker comprising one or more of a ring, band, and ink (e.g. platinum, platinum-iridium, gold, nitinol, palladium) configured to permit fluoroscopic visualization.

### Handle

Generally, pulsed electric field devices described herein may comprise a handle configured to allow an operator to grasp and control one or more of the position, orientation, and operation of a pulsed electric field device. In some variations, a handle may comprise a grip (e.g., hand grip) and one or more actuators to permit translation and/or rotation of the first and second elongate bodies in addition to steering by an optional delivery catheter. For example, the actuator may comprise one or more of a button, gear, slide, knob, switch, and the like. The actuator may be coupled to a gear configured to operate the pulsed electric field device. Control of an expandable member, in some variations, may be performed by an expansion member (e.g., screw/rotation actuator, inflation actuator) of the handle. In some variations, the handle may be configured to control PEF energy delivery to the electrode array of an expandable member, using, for example, a handheld switch, and/or footswitch.

### Insulator

Generally, the pulsed electric field devices described herein may include one or more insulators configured to electrically isolate one more portions of the electrode array, expandable member, inflatable member, dilator, and/or elongate body of the pulsed electric field device from each other. In some variations, the insulator may comprise one or more of a poly(p-xylylene) polymer such (e.g. parylene C, parylene N), polyurethane (PU), polytetrafluoroethylene (PTFE), expanded PTFE (ePTFE), polyimide (PI), polyester, polyethylene terephthalate (PET), PEEK, polyolefin, silicone, copolymer, a ceramic, combinations thereof, and the like.

### Guidewire

Generally, the systems described herein may comprise one or more guidewires configured to be slidably disposed within a lumen of an elongate body of a pulsed electric field device. The guidewire may be configured to assist in advancement of the pulsed electric field device through a gastrointestinal tract. In some variations, first and second elongate bodies of the pulsed electric field device may be translated along the guidewire relative to one another and/or the duodenum. In some variations, the guidewire may comprise one or more of stainless steel, nitinol, platinum, and other suitable biocompatible materials. In some variations, the guidewire may comprise a variable stiffness along its length. For example, a distal tip may be configured to be compliant (e.g., floppy) and an elongate body of the guidewire may be relatively stiff to aid pushability through patient anatomy. In some variations, a guidewire may comprise a diameter between about 0.36 mm and about 1.53 mm, and a length between about 180 cm and about 360 cm.

### Irrigation

Generally, the tissue treatment procedures using a pulsed electric field device as described herein may optionally comprise fluid delivery (e.g., fluid irrigation) during tissue treatment. In some variations, the tissue treatment procedures may benefit from fluid irrigation that may promote more reliable (e.g., consistent) electrical contact between the pulsed electric field device and tissue and therefore a more uniform electric field and an improvement to treatment outcomes. Fluid irrigation to tissue may further reduce tissue temperature through forced convention and may reduce arcing. Furthermore, fluid delivery may reduce the accumulation of electrically insulating corrosion and electrolysis products. In some variations, the fluid may function as a salt bridge between the electrodes and tissue that allows control of resistivity. In variations in which fluid is delivered, the fluid may be removed from (e.g., suctioned out of) a body cavity after applying the pulsed or modulated electric field. In some variations, the conductivity of the fluid introduced or removed may have an effect on the delivered therapy. For example, adding a solution that is less conductive than the tissue may facilitate more current being introduced into the tissue. Conductivity that is about the same as the tissue may facilitate a transfer of electric field energy into the tissue even if tissue contact between the electrodes and tissue is lacking. Finally, a fluid having a higher conductivity than the tissue may be removed.

In some variations, the pulsed electric field devices described herein may be configured to output fluid to irrigate tissue, such as duodenal tissue, of a patient. For example, an electrode array of a pulsed electric field device may engage the duodenum and may be configured to output fluid (e.g., saline), for example, where the electrodes contact tissue. The electrode array, for example, one or more electrodes of the electrode array, may output fluid between the electrode and tissue, which may directly target the electrodes and may allow a reduction in fluid volume. The electrode array may be energized to treat a predetermined portion of tissue to resurface the duodenum. Utilizing an electrode array that is configured to deliver fluid may eliminate the need for a separate irrigation device and/or system.

### Sensor

In some variations, the pulsed electric field devices and systems described here may comprise one or more sensors. Generally, the sensors may be configured to receive and/or transmit a signal corresponding to one or more parameters. In some variations, the sensor may comprise one or more of a temperature sensor, imaging sensor (e.g., CCD), pressure sensor, electrical sensor (e.g., impedance sensors, electrical voltage sensor, magnetic sensor (e.g., RF coil), electromagnetic sensor (e.g., infrared photodiode, optical photodiode, RF antenna), force sensor (e.g., a strain gauge), flow or velocity sensor (e.g., hot wire anemometer, vortex flowmeter), acceleration sensor (e.g., accelerometer), chemical sensor (e.g., pH sensors, protein sensor, glucose sensor), oxygen sensor (e.g., pulse oximetry sensor), audio sensor, sensor for sensing other physiological parameters, combinations thereof, and the like. In some variations, the electrical properties of cells can also be determined by applying an alternating current signal at a specific frequency to measure voltage.

Temperature measurements performed during a tissue treatment procedure may be used to determine one or more of tissue contact (e.g., complete contact, partial contact, no contact) with a pulsed field device and successful energy delivery to tissue. Thus, the safety of the tissue treatment procedures described herein may be enhanced through temperature measurement and monitoring. In some variations, temperature monitoring of the tissue may be used to prevent excess energy delivery to tissue that may otherwise lead to poor or suboptimal treatment outcomes. For example, energy delivery may be inhibited or delayed when tissue temperature measurements exceed a predetermined threshold.

### Suction Catheter

Generally, the suction catheters described herein may be configured to provide suction of tissue to an electrode array while facilitating visualization of the tissue and expandable member during a treatment procedure. For example, the suction catheter may be used with conventional visualization devices (e.g., endoscopes) and provide negative pressure (e.g., suction) through a lumen of the visualization device. The expandable member may comprise an electrode array and one or more fluid openings where the suction catheter may be configured to apply suction to tissue through the one or more fluid openings of the expandable member. The expandable member may comprise an expandable member lumen in an expanded configuration. The suction catheter may be configured to be received within the expandable member lumen to assist in applying suction through the one or more fluid openings of the expandable member. Accordingly, tissue contact with the expandable member may be improved while facilitating visualization of the procedure to ensure safety. For example, the suction catheter may be configured to advance from a distal end of a visualization device (e.g., endoscope) into the lumen of the expandable member (e.g., expandable member (520)) to provide efficient suction between the tissue and electrode array (e.g., electrode array (530)) while providing visualization of the tissue and pulsed electric field device (e.g., pulsed electric field device (500)) with a predetermined field-of-view. In some cases, the visualization device may be maintained in place relative to the expandable member when suctioning is performed. For example, an optical sensor of the visualization device may be positioned independently of the position of the suction ports of a suction catheter. The suction catheter may be translated from a lumen of the visualization device.

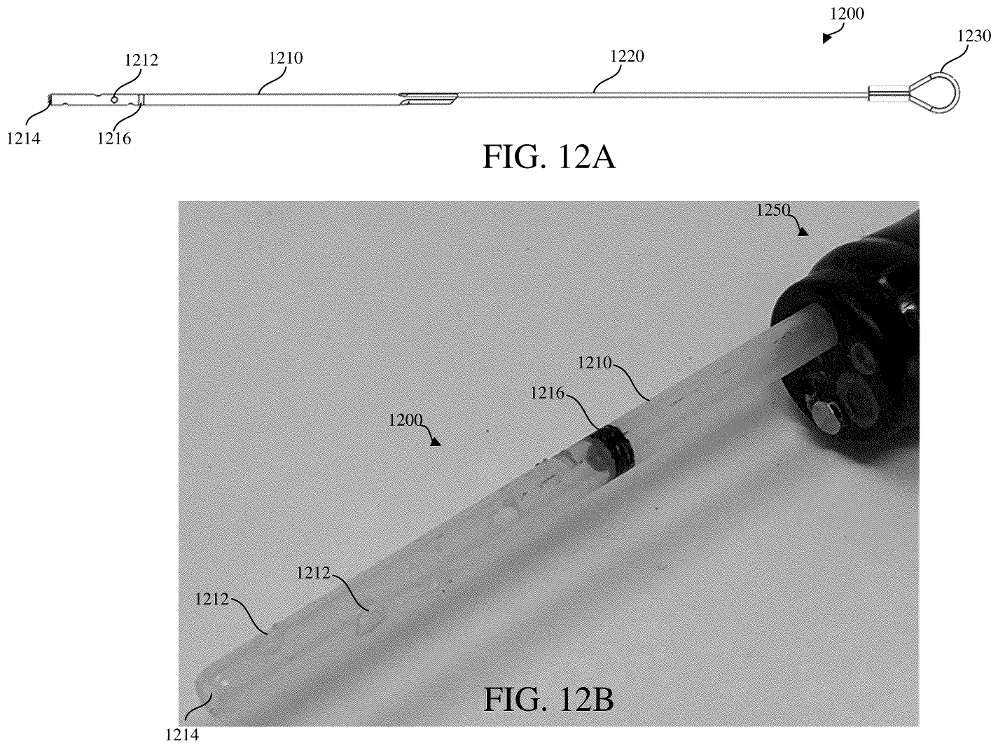

FIG. 12A is a side view of a suction catheter (1200) and FIG. 12B is a corresponding image of the suction catheter (1200) extending from a visualization device (1250). In some variations, the suction catheter (1200) may comprise a first elongate body (1210) coupled to a second elongate body (1220). In some variations, the first elongate body (1210) may include one or more apertures (1212), a proximal end, a distal end (1214), and a lumen between the proximal and distal ends. In some variations, the distal end (1214) of the first elongate body (1210) may comprise a plurality of apertures (1212) spaced around a circumference of the first elongate body (1210). The first elongate body (1210) may be advanced from a lumen of the visualization device (1250) to functionally extend a length of the visualization device lumen without altering a position of an optical sensor of the visualization device.

The second elongate body (1220) may be coupled to a proximal end of the first elongate body (1210). The second elongate body (1220) may comprise a stopper (1230) (e.g., loop, handle) configured to prevent the first elongate body (1210) from wholly translating out of the lumen of the visualization device and to minimize volume to allow maximum negative suction through the suction catheter. A diameter of the second elongate body (1220) may be less than a diameter of the first elongate body (1210).

The suction catheter (1200) may comprise a diameter configured to slidably translate through a lumen of the visualization device (1250). In some variations, the suction catheter (1200) and visualization device (1250) may be fluidically coupled to a negative pressure source (not shown for the sake of clarity). As shown in FIG. 12B, the suction catheter (1200) may be advanced from a lumen of the visualization device (1250) which may aid suction through, for example, a lumen of an expandable member. The negative pressure applied to the lumen of an expandable member is provided closer to the apertures of the expandable member such that negative pressure is applied more efficiently with reduced risk to the patient. In some variations, the suction catheter (1200) may be configured to apply suction one or more of radially therefrom and longitudinally therethrough. For example, the first elongate body (1210) may be atraumatic and comprise a plurality of apertures (1212). The plurality of apertures (1212) may be positioned in a configuration to provides radial and/or longitudinal suction. For example, the plurality of apertures may be spaced around a circumference of the first elongate body (1210) and/or may be at a distal end of the first elongate body (1210) to apply suction at least radially and/or longitudinally therethrough.

In some variations, the distal end (1214) of the first elongate body (1210) may have a distal opening and an atraumatic tip. In some variations, the plurality of apertures (121) may comprise opposing pairs of apertures and a distal opening. For example, aperture pairs may be provided on opposing sidewalls of the first elongate body (1210). In some variations, the apertures (1212) may be spaced around a circumference of the first elongate body (1210) to facilitate radial suction. For example, adjacent apertures along a length of the first elongate body (1210) may be offset from each other by a predetermined angle of about 15°, about 30°, about 45°, about 60°, about 75°, about 90°, including all ranges and sub-values in-between. In some variations, the apertures may be spaced apart along a length of the first elongate body (1210) at a distance of at least about 0.1 mm, at least about 0.5 mm, at least about 1 mm, at least about 1.5 mm, at least about 2.0 mm, at least about 2.5 mm, at least about 3.0 mm, at least about 5.0 mm, and at least about 10.0 mm, including all ranges and sub-values in-between. In some variations, the apertures may be offset from each other along a length of the first elongate body by about 90°, and be spaced apart from each other by at least about 1 mm. Each aperture may comprise a surface area of up to about 0.6 mm², up to about 0.5 mm², up to about 0.3 mm², up to 0.2 mm², including all ranges and sub-values in-between.

In some variations, a diameter of each aperture of the plurality of apertures (1212) may be equal to or less than an inner diameter of the suction catheter (1200). In some variations, a ratio of an aperture diameter to an inner diameter of the first elongate body (1210) may be between about 1:1 and about 1:5. In some variations, the suction catheter (1200) may comprise a length of at least 190 cm.

In some variations, the each of the plurality of apertures (1212) may have a shape including one or more of a circle, ellipse (e.g., oval), polygon (e.g., triangle, square, pentagon, hexagon, octagon), combinations thereof, and the like. For example, a minor axis of the ellipse may be equal to or less than an inner diameter of the suction catheter lumen and the major axis may be greater than the inner diameter of the suction catheter lumen. Apertures having an ellipse shape may have a greater surface area than circular apertures. The number of apertures may be an even number or an odd number, an even number of pairs or an odd number of pairs. Each of the apertures of the plurality of apertures may have the same shape and size or different shapes and/or sizes. The spacing between apertures may be the same or different along a length of the first elongate body, and the spacing between apertures around a circumference of the first elongate body may be the same or different. In some variations, apertures of different shapes and sizes may be preferentially positioned in predetermined locations to apply greater negative pressure to different portions of the suction catheter. For example, distal apertures may have a larger surface area than proximal apertures, the number of distal apertures may be greater than a number of proximal apertures, and a spacing between the distal apertures may be less than the spacing between proximal apertures.

In some variations, a sidewall of the first elongate body (1210) may have a thickness of between about 0.3 mm and about 0.7 mm, between about 0.4 m and about 0.6 mm, between about 0.45 mm and about 0.55 mm, and about 0.5 mm, including all ranges and sub-values in-between.

In some variations, the first elongate body (1210) and/or the second elongate body (1220) may comprise one or more fiducial markers (1216) (e.g., 2, 3, 4, 5 or more) configured to aid alignment of the suction catheter to the expandable member (first elongate body) or the visualization device (second elongate body) during a treatment procedure. In some variations, the fiducial maker(s) (1216) may be disposed on an external surface of the first elongate body (1210) proximal to the most proximal aperture of the plurality of apertures (1212). In some variations, the operator may align the position of the fiducial marker with a plane of a proximal edge of the expandable member. Additionally or alternatively, the fiducial marker(s) may be disposed on an external surface of the second elongate body (1220), such as, for example, near a proximal end of the second elongate body (1220).

In some variations, the suction catheter (1200) may be advanced from a lumen of the visualization device (1250) until the fiducial marker (1216) is aligned with a plane formed by a proximal edge of an expandable member. In this manner, the suction applied through the plurality of apertures (1212) at a distal end of the first elongate body (1210) may be efficiently directed at the tissue in contact with the expandable member. In some variations, a fiducial maker (1216) may be disposed from the distal end (1214) of the first elongate body (1210) at a distance of between about 1 mm and about 50 mm, between about 10 mm and about 50 mm, between about 20 mm and about 40 mm, between about 20 mm and about 30 mm, about 20 mm, and between about 30 mm and about 50 mm, including all ranges and sub-values in-between.

Additionally or alternatively, the suction catheter (1200) may be advanced from a lumen of the visualization device (1250) until a fiducial marker on a proximal end of the second elongate body (1220) is aligned with a distal end of the visualization device. In some variations, a fiducial maker of the second elongate body may be disposed from the distal end of the second elongate body (1220) at a distance of between about 1 mm and about 100 mm, between about 10 mm and about 100 mm, between about 20 mm and about 80 mm, between about 20 mm and about 60 mm, about 20 mm, and between about 40 mm and about 60 mm, including all ranges and sub-values in-between.

In some variations, the second elongate body (1220) may comprise one or more of a wire, cable, coil, braid, and the like and/or may otherwise be absent a lumen therethrough. Put differently, the second elongate body (1220) may be solid. A diameter of the second elongate body (1220) may be less than a diameter of the lumen of the first elongate body (1210) in order to maximize the negative pressure applied at a distal end of the suction catheter. Alternatively, the second elongate body may define a lumen. The first elongate body (1210) may be directly coupled distal to the second elongate body (1220). For example, as shown in FIG. 12A, a distal end of the second elongate body (1220) may be coupled to an inner surface of the lumen of the first elongate body (1210) at a proximal end of the first elongate body (1210). For example, the second elongate body (1220) may comprise a stainless steel cable (e.g., 49 strand).

In some variations, a proximal end of the second elongate body (1210) may be configured to restrict translation of the suction catheter (1200) through the visualization device (1250) such that advancement of the first elongate body (1210) from a distal end of the visualization device (1250) may be limited. For example, a proximal end of the second elongate body (1220) may comprise a stopper (1230) where a diameter of the stopper (e.g., loop) is larger than a diameter of the lumen of the visualization device (1250). The stopper (1230) may be disposed proximal to a proximal end of the visualization device (1250) such that when the loop (1230) begins to enter the lumen of the visualization device (1250), the stopper (1230) will physically prevent the suction catheter (1200) from advancing further relative to the visualization device (1250). The stopper (1230) or other similar structure may prevent loss of the suction catheter (1200) within a body cavity of the patient.

Additionally or alternatively, the pulsed electric field device (500) may be configured to provide suction of tissue to an electrode array of an expandable member. For example, the pulsed electric field device (500) may comprise a suction catheter coupled (e.g., using a clip, sheath) to the second elongate body (560) where the suction catheter is generally disposed parallel to the first elongate body (510). For example, the suction catheter or a portion of the suction catheter may be configured to translate relative to the lumen of the expandable member to provide suction. In some variations, the first elongate body (510) of the pulsed electric field device (500) may comprise a suction lumen configured to provide suction of tissue to an electrode array of an expandable member. For example, the first elongate body (510) may comprise an inner shaft and an outer shaft disposed around the inner shaft, where suction is provided through the outer shaft. In some variations, the second elongate body (560) may be composed of a flexible polymeric material such as Teflon, Nylon, Pebax, urethane, combinations thereof, and the like.

### Multiplexor

Generally, the multiplexors described herein may be configured to provide energy (e.g., PEF energy waveforms) to a pulsed electric field device to treat target tissue. For example, a signal generator as described herein throughout may comprise, or be operatively coupled to, a multiplexor configured to distribute a pulsed or modulated electric field waveform generated by the signal generator to one or more sections (e.g., zones, portions, groups, subsets) of the electrode array.

In some variations, the multiplexor may comprise an electric circuit including one or more switches. One or more of the switches may be electrically coupled to predetermined sections of the electrode array. For example, the multiplexor may be configured to independently actuate each switch. Accordingly, the multiplexor may be configured to deliver an electrical signal (e.g., pulse waveform) to a predetermined section of the electrode array. In some variations, the multiplexor may be configured to provide a pulse waveform to one or more sections of the electrode array simultaneously. In some variations, the multiplexor may be configured to provide a pulse waveform to one or more sections of the electrode array asynchronously. For example, the pulse waveform may be delivered asynchronously to one or more sections according to a predetermined sequence.

In some variations, the predetermined sequence may be modified based on the number of sections that in contact with tissue. For example, the expandable member may be configured to expand radially until the expandable member reaches a diameter that corresponds to a diameter of a body cavity or lumen, such as, for example, the GI tract (e.g., duodenum). The expandable member may continue expanding radially to dilate (e.g., stretch) the tissue, such as, for example, a predetermined amount. In some variations, expansion of the expandable member may be limited to prevent damage to tissue due to dilation. The dimensions of a body cavity or lumen (e.g., diameter, tissue thickness) may vary between patients. Therefore, the number of sections of the electrode array that may be in contact with tissue when in an expanded configuration may vary between patients. In some variations, a section of the electrode array may not be included in a predetermined sequence if substantially all of the electrodes of the section are not in contact with tissue. Advantageously, activating a section with substantially all of the electrodes in contact with tissue may avoid electrical shorting. Accordingly, the number of sections included in the predetermined sequence may vary.

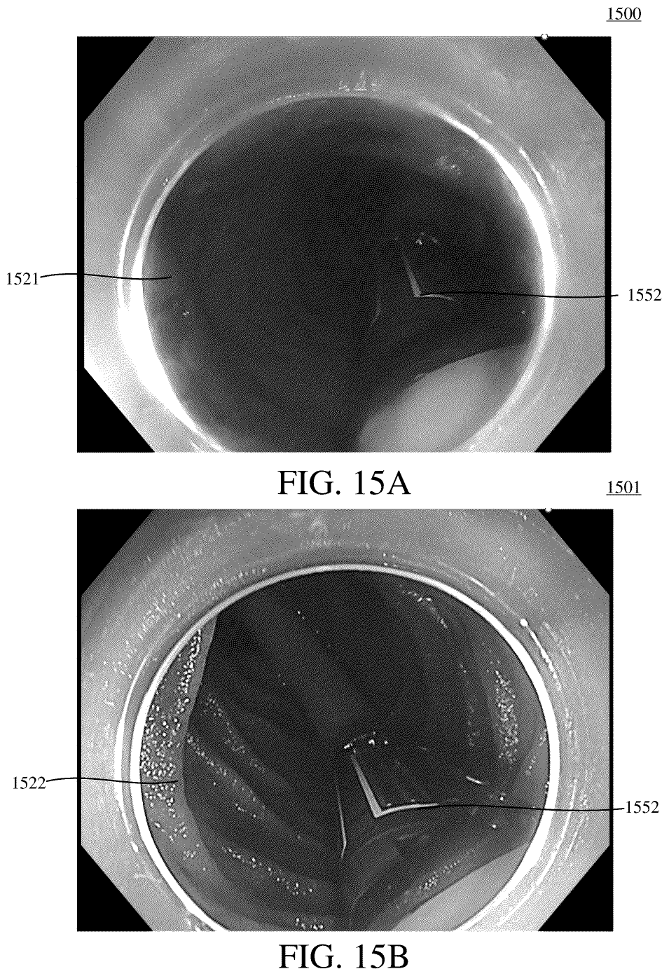

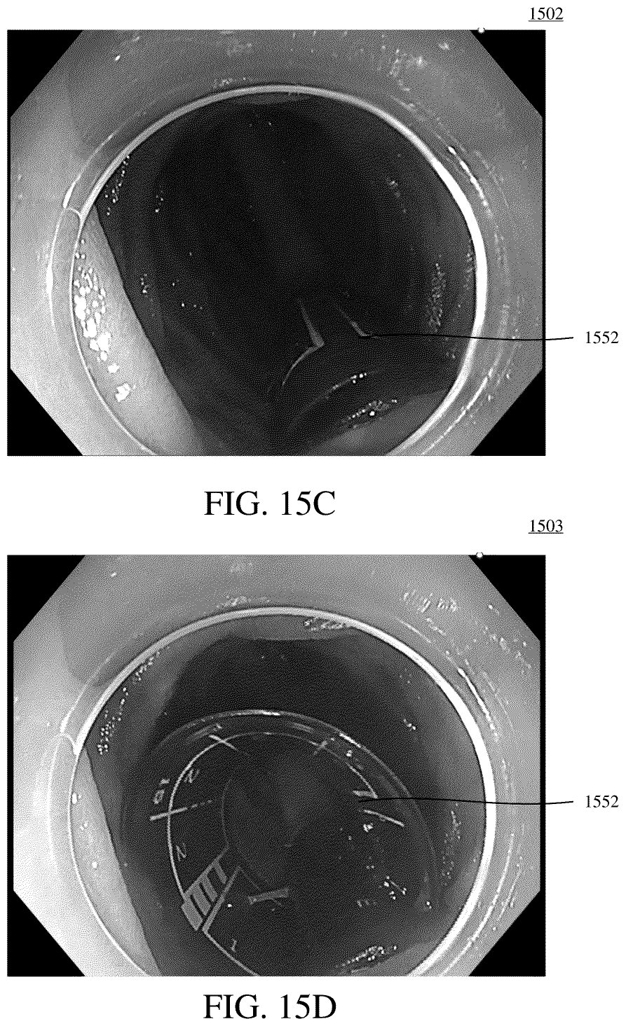

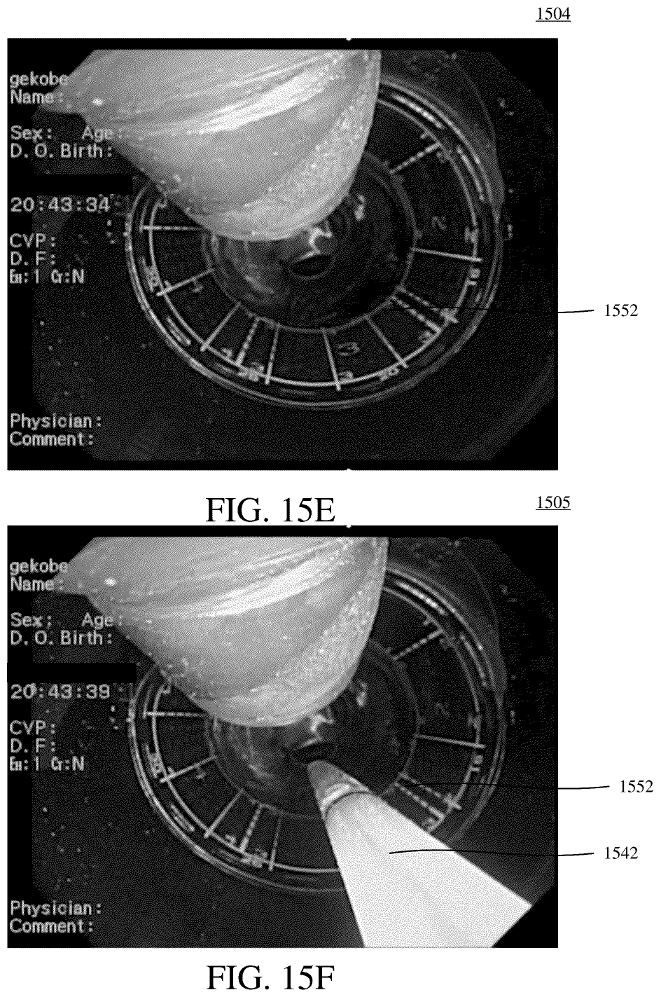

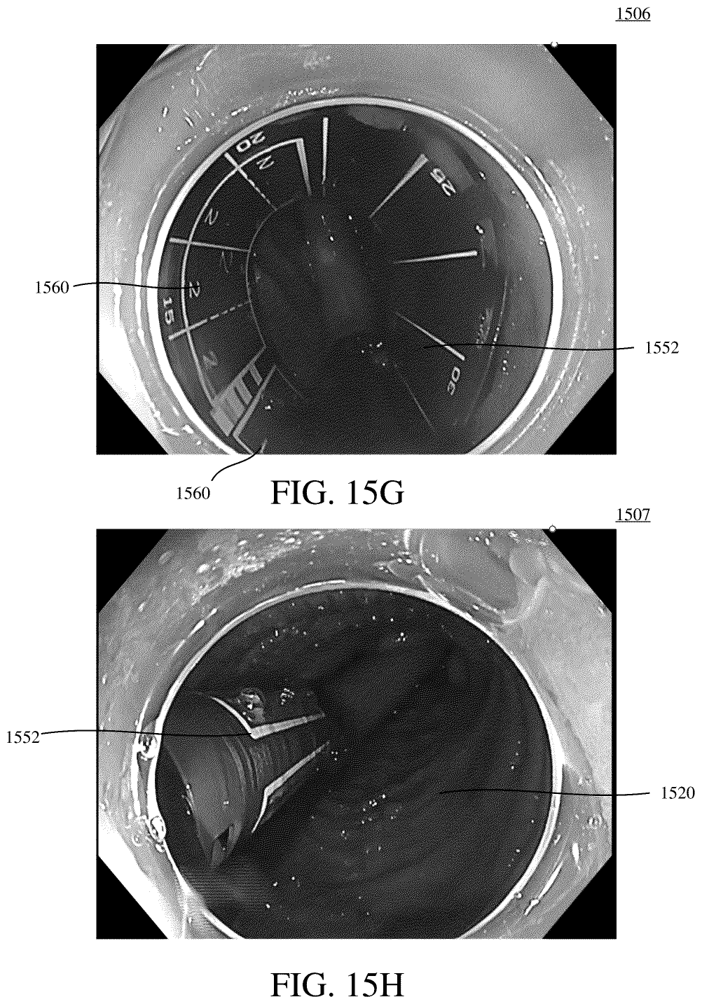

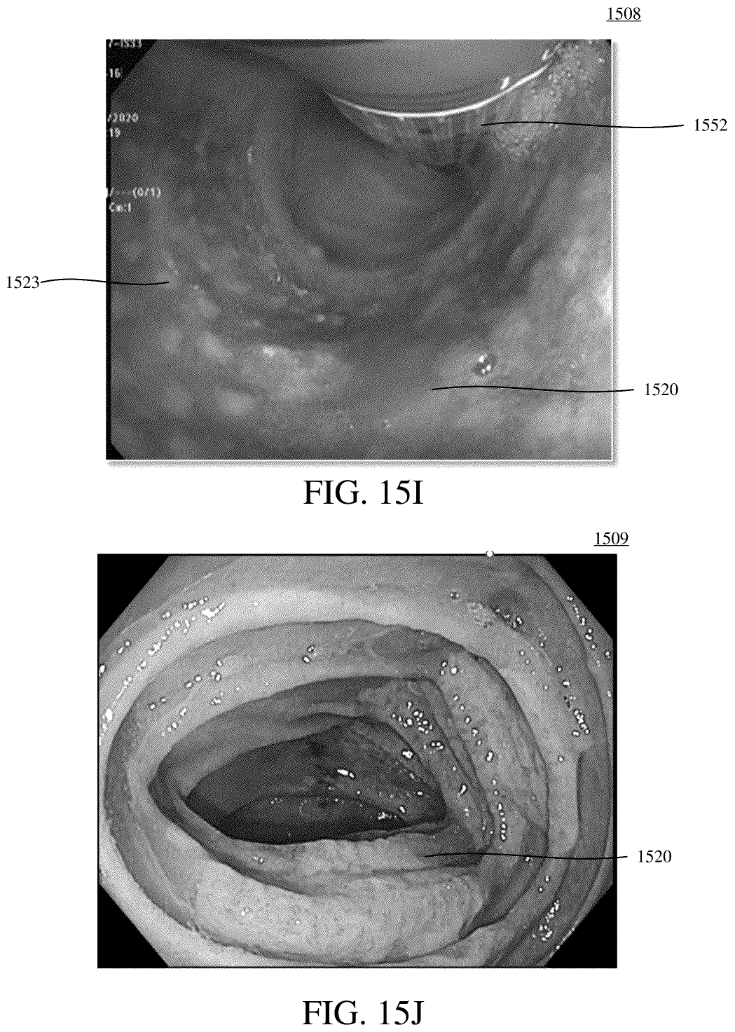

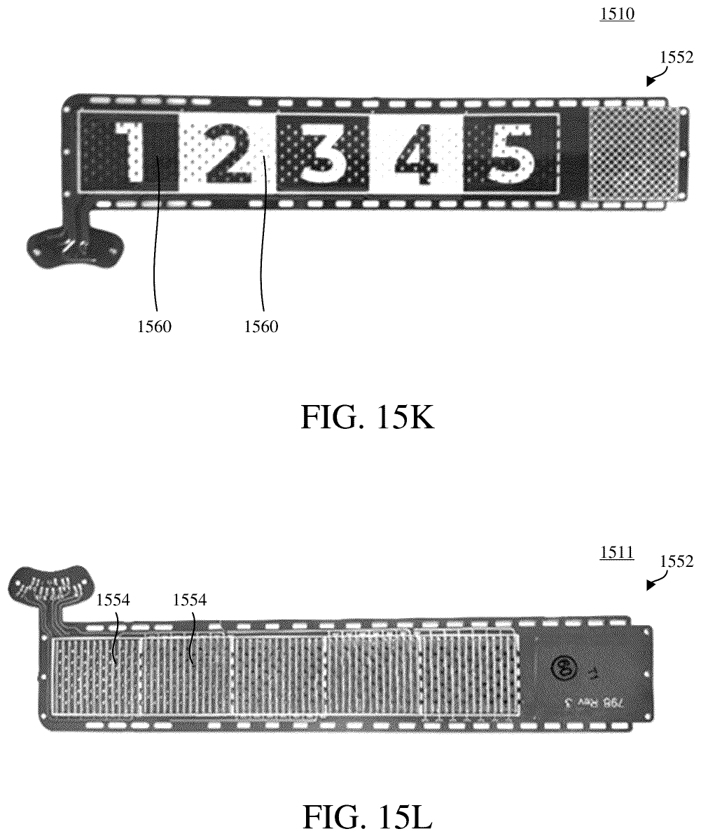

In some variations, the predetermined sequence may be modified to ensure that only the sections of the electrode array that are in substantial (e.g., full) contact with tissue are activated. For example, a visualization device may be used to visualize the expandable member, the electrode array and/or tissue as the expandable member transitions from an unexpanded configuration to an expanded configuration. In some variations, an inner surface (e.g., surface opposite a tissue-facing surface) of the expandable member may comprise one or more fiducial markers corresponding to predetermined sections of the electrode array (e.g., section 1, section 2, section 3, section 4, section 5). For example, image (1506) of FIG. 15G shows fiducial markers (1560) along an inner surface of the expandable member (1552) indicating the portions of the expandable member corresponding to the numbered electrode sections. While five sections are shown, it should be appreciated that suitable number of sections may be employed (e.g., two, three, four, six, seven, eight or more). In some variations, tissue contact (e.g., engagement, dilation) between the expandable member (1552) and tissue may be confirmed visually (e.g., via endoscopic visualization) with respect to the markers (1560). In some variations, the fiducial markers (1560) may outline the boundaries of one or more electrode sections.

FIG. 15K is an image (1510) of an inner surface of an unrolled expandable member (1552) including a set of fiducial markers (1560). For example, fiducial markers (1560) (e.g., numbers and white boxes) correspond to an electrode section of the expandable member (1552). FIG. 15L is an image (1510) of an outer surface (e.g., tissue-facing surface) of an unrolled expandable member (1552) including a plurality of electrodes (1554) corresponding to the electrode sections indicated by the first fiducial markers (1560).

In some variations, the expandable member may comprise an electrode array with one or more electrode sections. For example, the electrode array may comprise between about one and about ten sections, between about one and about eight sections, between about one and about seven sections, between one and about six sections, between about one and about five sections, between about two and about five sections, and between about three and about five sections, including about one section, about two sections, about three sections, about four sections, about five sections, about six sections, about seven sections, about eight sections, about nine sections, and about ten sections, including all ranges and sub-values in-between.

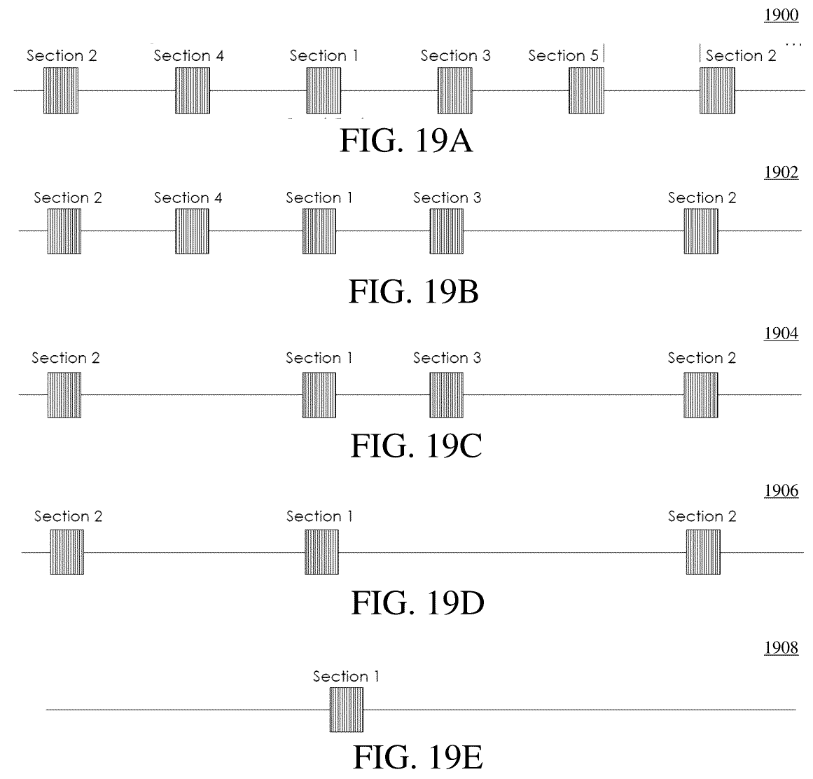

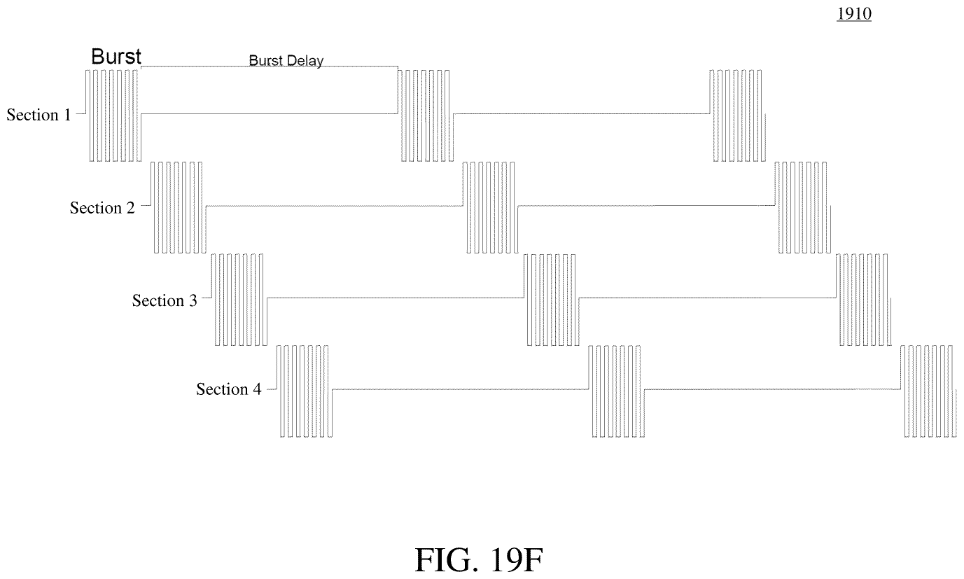

With continued reference to the exemplary expandable member comprising 5 electrode sections of FIG. 15K, as shown in the schematic diagram (1900) of FIG. 19A, Section 2 of an electrode array may be activated, followed subsequently by activation of Section 4, Section 1, Section 3, and Section 5 in an interleaved manner. The order of the electrode sections of the electrode array may be numerical (e.g., 1, 2, 3, 4, 5) such that Section 1 is proximate to Section 2 (e.g., adjacent to, shares a boundary or edge, in contact with) and non-proximate to Sections 3, 4, and 5 (e.g., non-adjacent, separated or spaced from, not in contact with). Similarly, Section 2 is proximate or adjacent to Sections 1 and 3, and non-proximate or non-adjacent to Sections 4 and 5. In some variations, deactivation of each section of a plurality of sections is applied independently. For example, one or more sections are not selected for energy delivery in diagrams (1902, 1904, 1906, 1908, 1910) of respective FIGS. 19B, 19C, 19D, 19E, and 19F where the predetermined sequence otherwise follows that of the diagram (1900) of FIG. 19A. For example, section 5 is not activated in FIG. 19B while the timing of energy delivery to sections 2, 4, 1, and 3 is unaffected. In FIG. 19C, sections 4 and 5 are not activated, but the timing of activation of sections 2, 1, and 3 is the same as in FIG. 19A. FIG. 19F further illustrates in the diagram (1910) that energy delivery (e.g., burst, burst delay) to each section is independent of the other sections such that energy delivery is interleaved. That is, the section burst delay is independent of the number of activated electrode sections such that the start of the second pulse waveform does not depend on the number of activated electrode sections. In this manner, the section burst delay for a section may not change even when one or more sections are not selected for activation. In some variations, the plurality of sections may comprise up to about ten sections. In some variations, the signal generator may be configured to repeat the predetermined sequence between about 5 and about 15 times. It will be apparent that a number of predetermined sequence variations may be provided such that non-proximate electrode sections are activated. For example, and with reference again to FIG. 15K, non-proximate activations may comprise, without limitation, activation of electrode sections 1-4-2-5-3; 5-2-4-1-3; 3-1-5-2-4; 4-2-5-1-3. All variations of non-proximate electrode sections are within the scope of the present disclosure.

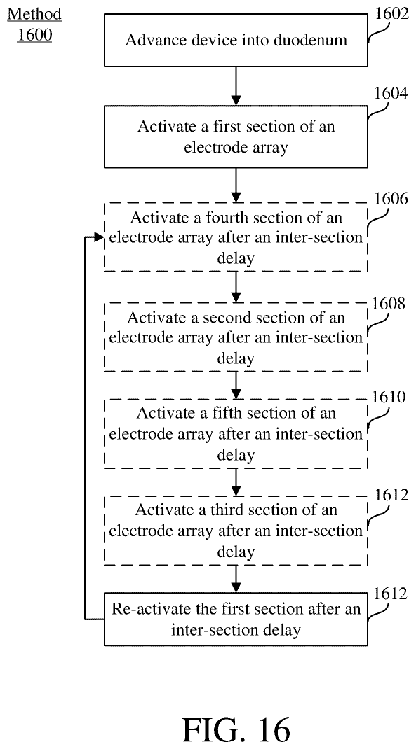

With continued reference to FIG. 16K, FIG. 16 provides a flow chart of an exemplary method 1600. Step 1602 comprises advancing of the device into the patient's duodenum. Step 1604 comprises activating a first electrode section of an electrode array. In a variation where a single electrode section is provided, the first electrode section may be re-activated as in step 1612 following an inter-section delay. Where more than one electrode sections are provided and activated, step 1606 comprises activating a fourth electrode section, wherein the fourth electrode section is not proximate or adjacent to the first electrode section. Step 1608 comprises activating a second electrode section, wherein the second electrode section is not proximate or adjacent to the fourth electrode section. Step 1610 comprises activating a fifth electrode section, wherein the fifth electrode section is not proximate or adjacent to the fourth electrode section. Step 1612 comprises activating a third electrode section, wherein the third electrode section is not proximate or adjacent to the fifth electrode section. Steps 1606-1612 are shown in dashed lines to illustrate a variation that comprises a single electrode section.

Further, in some variations, a predetermined or preprogrammed inter-section delay may not be required beyond the circuitry delay that results from activating a first electrode section for the predetermined time and the predetermined magnitude, then switching to activate a second, non-proximate or non-adjacent electrode section (for example, the fourth electrode section in FIG. 16) for the predetermined time and the predetermined magnitude. The natural delay in executing these steps, without an additional interposing inter-section delay, maybe sufficient to maintain temperature at an acceptable level in some variations.

### Pulse or Signal Generator

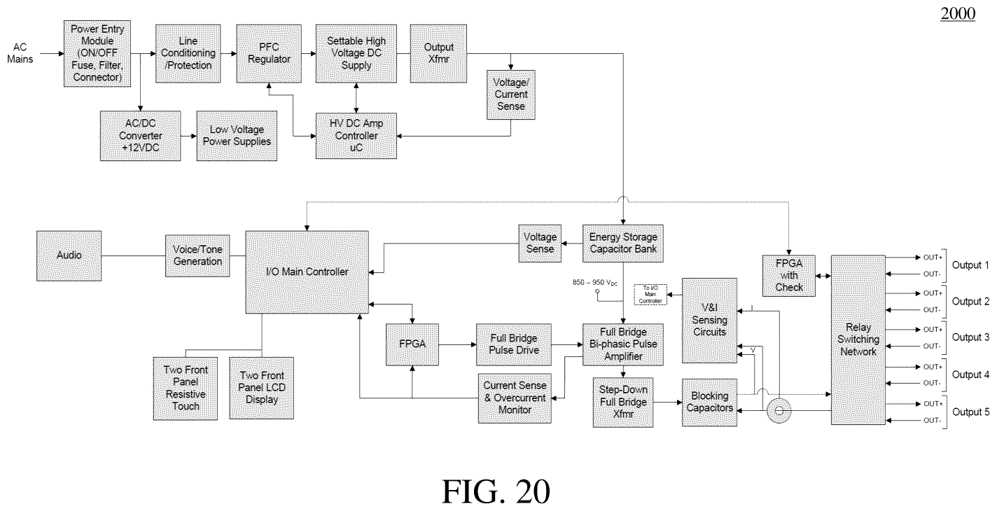

Generally, the systems described herein may include one or more signal generators configured to generate and deliver energy (e.g., PEF energy waveforms) to a pulsed electric field device to treat tissue. In some variations, a PEF system as described herein may include a signal generator having an energy source and a processor configured to deliver a waveform to deliver energy to tissue. In some variations, the signal generator may be configured to generate and deliver a plurality of signal types including, but not limited to, AC current, square wave AC current, sine wave AC current, AC current interrupted at predetermined time intervals, multiple profile current pulses trains of various power intensities, direct current (DC) impulses, stimulus range impulses, hybrid electrical impulses, combinations thereof, and the like. For example, the signal generator may be configured to generate one or more monophasic (DC) pulses and biphasic (DC and AC) pulses. FIG. 20 depicts a block diagram (2000) of an exemplary signal generator.

In some variations, a signal generator may be configured to generate a waveform between about 1 V and about 3,000 V, between about 100 V and about 2,000 V, between about 300 V and about 1,000 V, between about 500 V and about 900 V, between about 600 V and about 850 V, between about 715 V and about 825 V, between about 725 V and about 775 V, and between about 740 V and about 760 V, including about 100 V, about 200 V, about 300 V, about 400 V, about 500 V, about 600 V, about 700 V, about 750 V, about 800 V, about 1,000 V, about 1,100 V, about 1,200 V, about 1,300 V, about 1,400 V, about 1,500 V, about 1,600 V, about 1,700 V, about 1,800 V, about 1,900 V, about 2,000 V, including all ranges and sub-values in-between.

In some variations, the pulsed waveform may comprise a drive voltage at the electrode array between about 400 V and about 600 V, between about 400 V and about 550 V, between about 440 V and about 600 V, or between about 440 V and about 550 V, between about 5 kV and about 500 kV, between about 5 kV and about 15 kV, between about 5 kV and about 20 kV, between about 10 kV and about 20 kV, between about 15 kV and about 20 kV, including all values and sub-ranges in-between any of the aforementioned ranges.

In some variations, the pulsed waveform may produce a current through the tissue between about 0.6 A and about 100 A, between about 1 A and about 75 A, between about 20 A and about 60 A, between about 30 A and about 50 A, or between about 36 A and about 48 A from the electrode array per square centimeter of the tissue, including all values and sub-ranges in-between any of the aforementioned ranges.

In some variations, the pulsed waveform may produce a pulsed or modulated electric field at the tissue, including all values and sub-ranges in-between any of the aforementioned ranges.

In some variations, the pulsed waveform may comprise a pulse width between about 0.5 μs and about 4 μs, between about 0.1 ns and about 1000 ns, between about 1 ns and about 100 ns, between about 1 ns and about 500 ns, between about 500 ns and about 1000 ns, between about 200 ns and about 800 ns, between about 400 ns and about 600 ns, including all values and sub-ranges in-between any of the aforementioned ranges.

As another example, the pulsed waveform in some variations may comprise a drive voltage at the electrode array between about 5 kV and about 500 kV, between about 5 kV and about 15 kV, between about 5 kV and about 20 kV, between about 10 kV and about 20 kV, between about 15 kV and about 20 kV, including all values and sub-ranges in-between any of the aforementioned ranges. In some variations, the pulsed waveform may comprise an amplitude of at least 10 kV/cm.

In some variations, a signal generator may be configured to generate a waveform having a current delivered into a system resistance of between about 1 A and about 200 A, between about 2Ω and about 30Ω, between about 5Ω and about 20Ω, between about 10Ω and about 20Ω, between about 11Ω and about 18Ω, and between about 12.75Ω and about 16.25Ω, including all ranges and sub-values in-between. For example, in some variations, the system resistance may be about 2Ω, about 5Ω, about 10Ω, about 11Ω, about 12Ω, about 12.75Ω, about 13Ω, about 14Ω, about 14.5Ω, about 15Ω, about 16Ω, about 16.25Ω, about 17Ω, and about 18Ω, including all ranges and sub-values in-between.

In some variations, a signal generator may be configured to generate a waveform having a frequency of between about 50 kHz and about 950 kHz, between about 100 kHz and about 900 kHz, between about 200 kHz and about 800 kHz, between about 300 kHz and about 800 kHz, between about 400 kHz and about 800 kHz, between about 500 kHz and about 800 kHz, between about 600 kHz and about 800 kHz, and between about 700 kHz and about 800 kHz, between about 0.1 Hz and about 10,000 Hz, between about 1 Hz and about 1,000 Hz, between about 1 Hz and about 100 Hz, between about 100 Hz and about 1,000 Hz, between about 1,000 Hz and about 5,000 Hz, between about 5,000 Hz and about 10,000 Hz, between about 2,000 Hz and about 8,000 Hz, and between about 4,000 Hz and about 6,000 Hz, including all values and sub-ranges in-between any of the aforementioned ranges.

It should be appreciated that any combination of energy parameters as disclosed herein may be used. For example, the pulsed waveform in some variations may comprise a frequency between about 50 kHz and about 950 kHz or between about 300 kHz and about 400 kHz, a drive voltage at the electrode array between about 400 V and about 600 V or between about 440 V and about 550 V, and produces a current through tissue between about 36 A and about 48 A from the electrode array per square centimeter of the tissue. The pulsed or modulated electric field at the tissue may be between about 2,000 V/cm and about 3,000 V/cm. In some variations, the pulsed waveform may comprise a set of about 50 pulses in groups of between about 8 and about 13, with a delay of between about 4 seconds and about 10 seconds between each group. In some variations, the pulsed or modulated electric field may be a therapeutic electric field at a compressed tissue depth of between about 0.25 mm and about 0.75 mm and/or an uncompressed tissue depth of between about 0.50 mm and about 1.5 mm. In some variations, the pulse waveform may comprise a pulse width between about 0.5 μs and about 4 μs.

Generally, more than about 1,000 V/cm to about 2,500 V/cm is required at a treatment depth of tissue to induce electric fields across cell membranes greater than about 0.5 V in tissue such as the duodenum. In some variations, more than about 1,500 to about 4,500 V/cm, including all ranges and sub-values in-between, is required at a treatment depth of tissue to induce electric fields across cell membranes greater than about 0.5 V in the duodenum. Even relatively low tissue conductivity (e.g., about 0.3 S/m) may generate bulk tissue heating rates of at least about 800° C./s. The maximum temperature rise that should occur may be about 8° C. such that a maximum continuous on-time (100% duty cycle of alternating polarity pulses) may be about 10 msec. For example, the pulse waveform may comprise pairs of unipolar pulses of about 1 μs in groups between about 5 and about 500, with a delay between each group. In some of these variations, the pulse waveform may comprise a group delay between about 10 μs and about 4000 μs, and an intersection delay (e.g., replenish rate) of between about 50 ms and about 4000 ms, including all ranges and sub-values in-between. In some variations, a series of these groups may be repetitively applied with increasingly longer delays between series. In some variations, a sequence of series may be applied with longer delays between sequences. In some variations, about 15 milliseconds of cumulative ON time may be distributed across about 10 seconds.

In some variations, the signal generator may be configured to generate a waveform having a current, a voltage, and a power in the pulsed or modulated electric field spectrum between about 250 kHz and about 950 MHz, a pulse width between about 0.5 μs and about 4 μs, a voltage applied by the electrode array of between about 100 V and about 2 kV, and a current density between about 0.6 A and about 100 A from the electrode array per square centimeter of tissue. In some variations, the signal generator may be configured to drive into tissue resistance of from about 5Ω to about 30Ω of load. For example, the current density may be between about 0.6 A and about 100 A from the electrode array per square centimeter of tissue.

In some variations, the pulse waveform may comprise a pulse group of between about 1 and about 50, between about 1 and about 25, between about 1 and 10, between about 5 and 45, between about 10 and 40, between about 20 and 30, and between about 30 and 50, including all ranges and sub-values in-between. Each group of pulses may have between about 1 pulse and about 500 pulses, between about 1 pulse and about 100 pulses, between about 1 pulse and about 200 pulses, between about 1 pulse and about 300 pulses, between about 1 pulse and about 400 pulses, between about 100 pulses and about 500 pulses, between about 200 pulses and about 500 pulses, between about 300 pulses and about 500 pulses, between about 400 pulses and about 500 pulses, and between about 400 pulses and about 500 pulses, including all ranges and sub-values in-between. In some variations, the pulsed waveform may comprise between about 5 groups and about 20 groups or between about 8 groups and about 13 groups, including all values and sub-ranges in-between any of the aforementioned ranges. In some variations, the pulsed waveform may comprise a delay between groups of between about 1 second and about 20 seconds, or between about 4 seconds and about 10 seconds, including all values and sub-ranges in-between any of the aforementioned ranges.

In some variations, a set of bipolar pulses may be divided into bursts of bipolar pairs with a time delay between the bursts. This may allow the heat generated at the cell membranes to disperse, allowing additional treatment before the transition from cell lysis to necrosis. The total time that pulsed or modulated electric field is applied to the tissue may determine the density and size of the membrane pores, and the extent that ion flow has altered the contents of a cell. For example, given a tissue thermal diffusivity κ of 0.13 mm²/s and a cell diameter D_{cell} of 10 micron, the thermal diffusion time my be approximated as D_{cell}²/κ=0.8 msec. Thus, applying a pulse burst and then waiting a millisecond allows the temperature to equilibrate across the cell. For example, a balanced bipolar pulse waveform (e.g., within 10%) may reduce sympathetic nerve excitation, which may reduce perceived pain and spontaneous muscle contraction. In some variations, microsecond pulsing between about 1 μs and about 10 μs may generate cell lysis while minimizing nerve stimulation. An electric field distribution produced by short bipolar pulses does not depend as strongly on tissue homogeneity especially in anisotropic areas.

In some variations, the pulse waveform (i.e., pulsed or modulated electric field waveform) may be generated by the signal generator. The pulse waveform may be delivered to an electrode array such that the electrode array may generate an electric field. In some variations, the depth of treatment may be affected by the size and/or spacing of one or more electrodes of the electrode array and parameters of the pulse waveform. For example, the pulse waveform described herein may comprise one or more pulses. Each pulse may comprise a square, a triangle, a rectangle, or any other shape. In some variations, the pulse may comprise a square shape. In some variations, each pulse may comprise a bipolar pulse. Each pulse may comprise a series (i.e., burst) of pulses. For example, the series of pulses may comprise between about 1 pulse and about 500 pulses, between about 10 pulses and about 90 pulses, between about 20 pulses and about 80 pulses, between about 30 pulses and about 70 pulses, between about 40 pulses and about 60 pulses, and between about 45 pulses and about 55 pulses, between about 50 pulses and about 500 pulses, between about 100 pulses and about 500 pulses, between about 200 pulses and about 500 pulses, between about 300 pulses and about 500 pulses, between about 400 pulses and about 500 pulses, between about 10 pulses and about 200 pulses, between about 10 pulses and about 100 pulses, including about 1 pulse, about 10 pulses, about 15 pulses, about 20 pulses, about 30 pulses, about 40 pulses, about 45 pulses, about 50 pulses, about 55 pulses, about 60 pulses, about 70 pulses, about 80 pulses, about 90 pulses, about 100 pulses, including all ranges and sub-values in-between. In some variations, the series of pulses may comprise about 50 pulses.

Pulsed electric field waveforms may be delivered to an electrode array where, for example, two or more non-proximate sections of the plurality of sections of the electrode array receive the waveform in a predetermined sequence (e.g., of different groups of pulses for different sections) in order to increase safety and/or reduce unintended damage to the tissue by reducing a temperature increase in tissue. In some variations, the predetermined sequence described herein may comprise delivering a series of pulses per activation of a given section. Accordingly, the inter-section delay may correspond to the time interval between an end of a first series of pulses delivered to a first section and a start of a second series of pulses delivered to a second section. The intra-section delay may correspond to the time interval between an end of the first series of pulses delivered to a first section and a start of the second series of pulses delivered to the first section.

FIG. 17A is a schematic diagram (1700) of a pulse waveform. In some variations, each pulse of a pulse waveform may comprise a pulse width (Tₚ). In some variations, a pulse width Tₚ may correspond to the time between adjacent maxima or minima of a wave. In some variations, the pulse width Tₚ may be measured between zero-crossings that correspond to a full wave. In some variations, a set voltage Vₛₑₜ may correspond to the amplitude of the pulse measured in volts. In some variations, the pulse width may be inversely proportional to a frequency (f) of the pulse waveform, as given by equation (2):

$${T}_{p} = \frac{1}{f} \qquad equation ( 2 )$$

As shown in FIG. 17A, the pulse width may include a first time interval (T_{H}) corresponding to a positively-charged portion of a bipolar pulse and a second time interval (T_{L}) corresponding to a negatively-charged portion of a bipolar pulse. In some variations, the first time interval (T_{H}) and second time interval (T_{L}) may be equivalent.

In some variations, the pulse may comprise a square or rectangular shape comprising one or more phases. Each phase may be generated by a portion of an electric circuit, such as an H-bridge, of the signal generator. For example, as shown in FIG. 17A, a pulse may comprise a first phase 1710 (“Phase 1”) and a second phase 1720 (“Phase 2”). The first phase 1710 may be generated by a first portion of the H-bridge and the second phase 1720 may be generated by a second portion of the H-bridge. In some variations, the phases may be generated in parallel, such that net-zero points of a given phase may correspond to a non-zero point of another phase. The net-zero points of a given phase may correspond to an interval of zero energy. The waveforms of each phase may comprise one or more parameters. For example, as illustrated, the phase(s) may comprise the first time interval Tₕ, the pulse width Tₚ, the non-zero time interval T_{dt} (e.g., dead time), and a second time interval T_{L}. The Tₕ value may correspond to the interval of the waveform at a positive maxima. The T_{L} value may correspond to the interval of the waveform at a negative minima. The Tₚ value may correspond to the time between adjacent maxima or minima of a wave.

In some variations, the pulse width may be between about 1 μs to about 10 μs, between about 5 μs to about 10 μs, between about 3 μs to about 7 μs, between about 1.5 μs to about 4 μs, between about 2 μs to about 3.5 μs, between about 2.5 μs to about 3.25 μs, between about 2.7 μs to about 3 μs, and between about 2.8 μs to about 2.9 μs, including 1 μs, about 1.5 μs, about 2 μs, about 2.5 μs, about 2.6 μs, about 2.7 μs, about 2.8 μs, about 2.82 μs, about 2.84 μs, about 2.86 μs, about 2.88 μs, about 2.9 μs, about 3 μs, about 3.1 μs, about 3.25 μs, about 3.5 μs, about 4 μs, or about 5 μs. In an exemplary variation, the pulse width may be about 2.86 μs.

In some variations, the first time interval (T_{H}) and/or the second time interval (T_{L}) may each be between about 0.5 μs and about 2 μs, between about 1 μs and about 2 μs, between about 1.1 μs and about 1.9 μs, between about 1.2 μs and about 1.7 μs, between about 1.3 μs and about 1.5 μs, and between about 1.4 μs and about 1.5 μs, including about 0.5 μs, about 1 μs, about 1.1 μs, about 1.2 μs, about 1.3 μs, about 1.4 μs, about 1.41 μs, about 1.42 μs, about 1.43 μs, about 1.44 μs, about 1.45 μs, about 1.5 μs, and about 1.6 μs, including all ranges and sub-values in-between. In some variations, the first-time interval (T_{H}) and the second time interval (T_{L}) may each be about 1.43 μs.

In some variations, the frequency may be between about 50 kHz and about 950 kHz, between about 100 kHz and about 900 kHz, between about 200 kHz and about 500 kHz, between about 300 kHz and about 400 kHz, and between about 325 kHz and about 375 kHz, including about 100 kHz, about 200 kHz, about 300 kHz, about 325 kHz, about 350 kHz, about 375 kHz, about 400 kHz, and about 500 kHz, including all ranges and sub-values in-between. In an exemplary variation, the frequency may comprise about 350 kHz.

As shown in FIG. 17A, the signal generator described herein may be configured to switch from the positive portion to the negative portion with a non-zero time interval (T_{dT}) therebetween comprising a net-zero charge. Advantageously, a non-zero time interval of a net-zero charge may reduce risks associated with electrical shorting and/or electrical cross-talk within one or more components of the signal generator. In some variations, the non-zero time interval (T_{dT}) may be between about 0.01 μs and about 0.1 μs, between about 0.02 μs and about 0.1 μs, between about 0.03 μs and about 0.1 μs, between about 0.04 μs and about 0.1 μs, between about 0.05 μs and about 0.1 μs, between about 0.06 μs and about 0.1 μs, between about 0.07 μs and about 0.1 μs, and between about 0.08 μs and about 0.09 μs, including about 0.01 μs, about 0.02 μs, about 0.03 μs, about 0.04 μs, about 0.05 μs, about 0.06 μs, about 0.07 μs, about 0.08 μs, about 0.085 μs, about 0.086 μs, and about 0.09 μs, including all ranges and sub-values in-between. Alternatively, the pulse waveform may not include a non-zero time interval (T_{dT}). In some variations, the non-zero time interval (T_{dT}) corresponds to the first time interval (T_{H}), second time interval (T_{L}), and pulse width (Tₚ) as given by equation (3).

$${T}_{p} = {T}_{H} + {T}_{L} + 2 {T}_{dT} \qquad equation ( 3 )$$

As shown in the schematic diagram (1750) of FIG. 17B, the series or group of pulses described herein may comprise a burst time (T_{b}) or group time representing the time between a start of the series and an end of the series of pulses (e.g., a group of pulses). In some variations, the burst time (T_{b}) may correspond to the pulse width (Tₚ) and a number of pulses (nₚ) per series as given by equation (4) below. As described in more detail herein, a first group of pulses may correspond to a first pulse waveform delivered to a first section of an electrode array, and a second group of pulses may correspond to a second pulse waveform delivered to a second section of an electrode array. As described herein, a series of pulses may be applied to an electrode section of an electrode array. The burst time (T_{b}) may be determined using equation (4).

$${T}_{b} = {n}_{p} * {T}_{p} \qquad equation ( 4 )$$

In some variations, the series or group of pulses (e.g., bipolar pulsed electric field waveforms) may comprise between about 1 and about 100 pulses, between about 10 pulses and about 90 pulses, between about 20 pulses and about 80 pulses, between about 30 pulses and about 70 pulses, between about 40 pulses and about 60 pulses, between about 45 pulses and about 55 pulses, between about 40 pulses and about 50 pulses, and between about 50 pulses and about 60 pulses, between about 50 pulses and about 500 pulses, between about 100 pulses and about 500 pulses, between about 200 pulses and about 500 pulses, between about 300 pulses and about 500 pulses, between about 400 pulses and about 500 pulses, between about 10 pulses and about 200 pulses, between about 10 pulses and about 100 pulses, including about 1 pulse, about 10 pulses, about 20 pulses, about 30 pulses, about 40 pulses, about 45 pulses, about 50 pulses, about 55 pulses, about 60 pulses, about 70 pulses, about 80 pulses, about 90 pulses, and about 100 pulses, including all ranges and sub-values in-between. In some variations, the series of pulses may comprise about 50 pulses.

In some variations, one or more of the electrode array sections may be energized (i.e., activated) according to a predetermined sequence using interleaved waveforms. For example, the sections may be activated successively (i.e., one section after another) such that successively activated sections are not proximate (e.g., immediately adjacent) to one another. For example, successively activated sections may be separated by at least one other section of the electrode array. In another example, successively activated sections may be separated by a non-conductive portion. In some variations, the predetermined sequence may comprise activating sections sequentially, such that proximate sections may be activated successively. In some variations, the sections may be wired and/or may be activated independently of one another. Alternatively, two or more sections may be activated concurrently. For example, non-proximate pairs of sections may be activated simultaneously to reduce a treatment time.

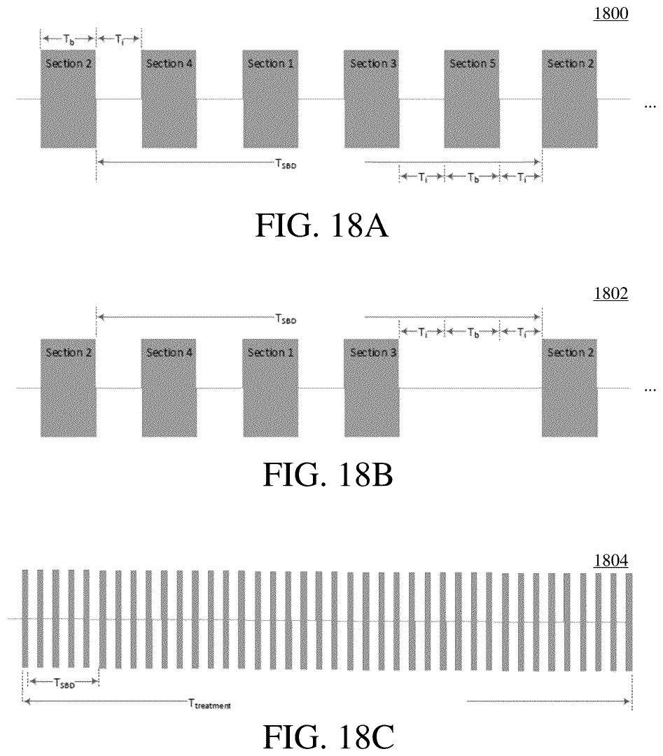

As shown in the schematic diagram (1800) of FIG. 18A, Section 2 of an electrode array may be activated, followed subsequently by activation of Section 4, Section 1, Section 3, and Section 5 in an interleaving manner. The section labeling shown in FIGS. 18A and 18B are exemplary and may be arranged in any order. For example, section 1 may be proximate (e.g., immediately adjacent) to section 2, which may be proximate to section 3, and so on. In some variations, the predetermined sequence may include a section burst delay (T_{SBD}), which may correspond to a time interval between the end of a burst of a given section and the beginning of a burst of that same section. In some variations, the section burst delay may be between about 1 second and about 20 seconds, between about 1 second and about 10 seconds, between about 1 second and about 5 seconds, between about 2 seconds and about 8 seconds, and between about 3 seconds and about 5 seconds, including all ranges and sub-values in-between.

In some variations, the predetermined sequence may be modified based on a selection of sections. For example, the predetermined sequence may initially be configured to activate every section (e.g., sections 1-5) of an electrode array. In some variations, a subset of the sections may be selected (e.g., by a user) or pre-programmed and a corresponding predetermined sequence may be modified to optimize the treatment. For example, a portion of tissue to be treated may have a diameter such that the tissue may be optimally dilated with four sections of the electrode array in contact with tissue and the fifth section of the electrode array not in contact with tissue. Accordingly, sections 1-4 of the electrode array may be selected for energy delivery and section 5 may be unselected, such that the unselected section is not activated.

As shown in schematic diagram (1800) of FIG. 18A, a predetermined sequence is shown where Sections 1-5 of an electrode array activated in a predetermined interleaved order (e.g., Section 2, Section 4, Section 1, Section 3, Section 5) using a first pulse waveform, and a second pulse waveform repeats the activation pattern of the first pulse waveform after an intra-section delay (T_{SBD}) (i.e., second delay). The pulse waveforms (e.g., first, second) may include one or more delays. For example, different sections (e.g., Section 2 followed by Section 4) may be activated following an inter-section delay (Tᵢ) (i.e., a first delay, an inter-section delay). In some variations, the delays (Tᵢ, T_{SBD}) may correspond to the burst time (T_{b}) and a number of pulses per series or group or burst (nₚ) as given in equation (5).

$${T}_{i} = \frac{{T}_{SBD} - ( {T}_{b} * ( {n}_{s} - 1 ) )}{{n}_{s}} \qquad equation ( 5 )$$

In some variations, the inter-section delay (Tᵢ) between pulse groups may be the same or different (e.g., an inter-section delay between Section 2 and 4 may be different from the inter-section delay between Sections 5 and 2). The intra-section delay (T_{SBD}) between different pulse waveforms may be the same or different.

In some variations, the number of activated sections of an electrode array may be based on one or more of the target tissue and/or chronic condition to be treated. In FIG. 18A, an electrode array includes five sections where every section of the electrode array is activated in the predetermined sequence. FIG. 18B depicts a schematic diagram (1802) where the predetermined sequence activates less than all sections of the electrode array in an interleaved manner. This may be useful where a smaller diameter of the expandable member is sufficient to treat target tissue such that, for example, only four of the five sections of the electrode array are activated and in contact with tissue. In some variations, deactivation of each section of a plurality of sections is applied independently. For example, Section 5 is not selected for energy delivery in diagram (1802) where the predetermined sequence otherwise follows that of the diagram (1800) of FIG. 18A. That is, the section burst delay may be independent of the number of activated electrode sections.

In some variations, activation of one or more sections of an electrode array may provide partial or full circumferential treatment of tissue. For example, a predetermined sequence may treat a circumference of tissue of up to about 360°, of up to about 330°, of up to about 300°, of up to about 270°, of up to about 240°, of up to about 210°, of up to about 180°, of up to about 150°, of up to about 120°, of up to about 90°, of up to about 60°, and of up to about 30°, including all ranges and sub-values in-between.

In some variations, the inter-section delay (Tᵢ) may comprise a time interval between about 10 ms and about 4 seconds, between about 50 ms and about 4 seconds, between about 100 ms and about 2 seconds, between about 200 ms and about 1 second, between about 300 ms and about 900 ms, between about 500 ms and about 850 ms, between about 600 ms and about 850 ms, between about 700 ms and about 850 ms, and between about 750 ms and about 850 ms, including about 50 ms, about 100 ms, about 200 ms, about 300 ms, about 400 ms, about 500 ms, about 600 ms, about 700 ms, about 800 ms, about 850 ms, about 1 second, about 2 seconds, about 3 seconds, and about 4 seconds, including all ranges and sub-values in-between. In some variations, an inter-section delay may comprise a time interval of about 800 ms or less.

In some variations, the intra-section delay may be different (e.g., shorter, longer) than the inter-section delay. In some variations, the intra-section and inter-section delays may be the same. In some variations, the intra-section delay may be between about 1 second and about 10 seconds, between about 1 second and about 8 seconds, between about 2 seconds and about 6 seconds, between about 3 seconds and about 5 seconds, and between about 3.5 seconds and about 4.5 seconds, including about 1 second, about 2 seconds, about 3 seconds, about 4 seconds, about 5 seconds, about 6 seconds, about 7 seconds, about 8 seconds, about 9 seconds, and about 10 seconds, including all ranges and sub-values in between. In some variations, an inter-section delay may be about 800 ms or less, between about 500 ms and about 1000 ms, or between about 500 ms and about 800 ms, including all ranges and sub-values in-between.

FIG. 18C depicts a diagram (1804) of a total treatment sequence including a plurality of pulse waveforms. The time interval from the beginning of the first pulse to the end of the last pulse may correspond to a cumulative treatment time Tₜᵣₑₐₜₘₑₙₜ. In some variations, the signal generator may be configured to activate the plurality of sections for a cumulative activation time between about 0.1 ms and about 10 ms over a treatment time between about 30 seconds and about 35 seconds, including all ranges and sub-values in-between. The total treatment sequence may comprise a plurality of pulse waveforms including up to about 50 pulse waveforms, up to about 40 pulse waveforms, up to about 30 pulse waveforms, up to about 20 pulse waveforms, up to about 15 pulse waveforms, up to about 10 pulse waveforms, and up to about 5 pulse waveforms, including all ranges and sub-values in-between. Any combination of the energy parameters described herein may be used and the treatment may be tailored to the particular target tissue and chronic condition being treated.

In some variations, the signal generator may be configured to control waveform generation and delivery in response to received sensor data. For example, energy delivery may be inhibited when a temperature sensor measurement confirms tissue temperature exceeding a predetermined threshold or ranges (e.g., above a predetermined maximum temperature). For example, energy delivery may be inhibited based on a temperature increase over a predetermined period of time (e.g., an increase of 2° C. over one second of time may inhibit further energy delivery).

In some variations, the signal generator may comprise a processor, memory, energy source (e.g., current source), and user interface. The processor may incorporate data received from one or more of the memory, the energy source, the user interface, and the pulsed electric field device. The memory may further store instructions to cause the processor to execute modules, processes and/or functions associated with the system, such as waveform generation and delivery. For example, the memory may be configured to store patient data, clinical data, procedure data, safety data, and/or the like.

Generally, the processor (e.g., CPU) of a signal generator described here may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor (e.g., temperature sensor) and a storage medium (e.g., memory, flash drive, memory card). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Generally, the pulsed electric field device described here may comprise a memory configured to store data and/or information. In some variations, the memory may comprise one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with a pulsed electric field device, such as signal waveform generation, pulsed electric field device control, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In some variations, the pulsed electric field device may further comprise a communication device configured to permit an operator to control one or more of the devices of the PEF system. The communication device may comprise a network interface configured to connect the pulsed electric field device to another system (e.g., Internet, remote server, database) by wired or wireless connection. In some variations, the pulsed electric field device may be in communication with other devices (e.g., cell phone, tablet, computer, smart watch, and the like) via one or more wired and/or wireless networks. In some variations, the network interface may comprise one or more of a radiofrequency receiver/transmitter, an optical (e.g., infrared) receiver/transmitter, and the like, configured to communicate with one or more devices and/or networks. The network interface may communicate by wires and/or wirelessly with one or more of the pulsed electric field device, network, database, and server.

The network interface may comprise RF circuitry configured to receive and/or transmit RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

In some variations, the user interface may comprise an input device (e.g., touch screen) and output device (e.g., display device) and be configured to receive input data from one or more of the pulsed electric field device, network, database, and server. For example, operator control of an input device (e.g., keyboard, buttons, touch screen) may be received by the user interface and may then be processed by processor and memory for the user interface to output a control signal to the pulsed electric field device. Some variations of an input device may comprise at least one switch configured to generate a control signal. For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive operator movement data from an optical sensor and classify an operator gesture as a control signal. A microphone may receive audio data and recognize an operator voice as a control signal.

A haptic device may be incorporated into one or more of the input and output devices to provide additional sensory output (e.g., force feedback) to the operator. For example, a haptic device may generate a tactile response (e.g., vibration) to confirm operator input to an input device (e.g., touch surface). As another example, haptic feedback may notify that operator input is overridden by the pulsed electric field device.

### II. Methods

Also described here are methods of treating tissue. In some variations, methods may comprise treating diabetes of a patient using the systems and devices described herein. In particular, the systems, devices, and methods described herein may resurface a predetermined portion of tissue, for example, duodenal tissue, for the treatment of, for example, diabetes using a pulsed or modulated (e.g., sine wave) electric field.

Generally, the methods of treating tissue may deliver pulsed or modulated electric field energy to remove native endothelial cell populations through non-thermal cell death that may address metabolic disorders such as, for example, obesity, Non-alcoholic fatty liver disease (NAFLD), Nonalcoholic steatohepatitis (NASH), Type I diabetes, and Type II diabetes. Gastric mucosal devitalization (GMD) without thermal injury to muscularis propria may modify one or more of serum ghrelin levels, triglycerides, HDL, relative weight loss, visceral adiposity, organ lipid content, liver lipid/protein ratio, gluconeogenesis, and liver lipid accumulation. Any of the methods described herein, such as energy delivery, may be performed using a monopolar or bipolar configuration in a body cavity or lumen of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. For example, energy delivery for treating Barrett's esophagus may provide long-term symptom management and reduce complications such as cancer. In some variations, precancerous esophageal cells may be treated while preserving healthy esophageal tissue.

In some variations, the suction may be applied at least radially and longitudinally by the suction catheter. In some variations, treating the target tissue treats one or more of a metabolic disorder, pre-cancer, cancer, proinflammatory processes, immunological processes. In some variations, the metabolic disorder may comprise one or more of obesity, Non-alcoholic fatty liver disease (NAFLD), Nonalcoholic steatohepatitis (NASH), Type I diabetes, and Type II diabetes. In some variations, the target tissue may comprise one or more of a duodenum, a pylorus, an esophagus, a stomach, a small intestine, and a large intestine. Gastric mucosal devitalization (GMD) without thermal injury to muscularis propria may modify one or more of serum ghrelin levels, triglycerides, HDL, relative weight loss, visceral adiposity, organ lipid content, liver lipid/protein ratio, gluconeogenesis, and liver lipid accumulation. Energy delivery may be performed using a monopolar or bipolar configuration. For example, energy delivery for treating Barrett's esophagus may provide long-term symptom management and reduce complications such as cancer. In some variations, precancerous esophageal cells may be treated while preserving healthy esophageal tissue. Any of the methods described herein may be performed in any portion of a body cavity or lumen of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like.

In some variations, the generated pulsed or modulated electric field may be substantially uniform such that pulsed or modulated electric field energy for tissue treatment may be delivered to a predetermined portion of tissue (e.g., mucosal layer of the duodenum) without significant energy delivery to deeper layers of the duodenum. Thus, the methods may improve the efficiency and effectiveness of energy delivery to duodenal tissue. Moreover, the methods described here may also avoid the excess thermal tissue heating necessarily generated by application of one or more other thermal energy modalities to tissue.

In some variations, methods may include applying suction to a pulsed electric field device in contact with tissue using a suction catheter. The suction catheter may, in some variations, be advanced from a lumen of a visualization device. For example, an expandable member having an electrode array may be in an expanded configuration for dilating tissue while the visualization device may be disposed proximal to expandable member to visualize the energy delivery. The suction catheter may be advanced into a lumen of the expandable member and may apply negative pressure to the tissue to further aid in a consistent tissue engagement with the expandable member and improved energy delivery. In some variations, energy delivery may include activating different sections of the electrode array in a predetermined order to minimize treatment time, an energy dose applied to tissue, and/or a temperature increase in the tissue. For example, non-proximate sections of the electrode array may be activated after an inter-section delay to generate a therapeutic electric field, minimize tissue temperature increase, and reduce electrical cross-talk.

### Method of Treating Tissue

Generally, methods of treating tissue may comprise generating a pulsed or modulated electric field to cause a change in tissue to treat one or more chronic condition, such as, for example, a metabolic disorder, pre-cancer, cancer, proinflammatory processes, immunological processes, and neurological disorders. For example, the metabolic disorder may comprise one or more of obesity, non-alcoholic fatty liver disease (NAFLD), nonalcoholic steatohepatitis (NASH), Type I diabetes, and Type II diabetes. In some variations, the tissue may include tissue from any body lumen or cavity such as any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like.

Normally, the small intestine sends signals to the brain, pancreas, and liver to promote glycemic hemostasis. For example, enteroendocrine cells of the mucosal villa may generate these signals. Duodenal mucosal resurfacing using the systems, methods, and devices described herein may be used to treat, for example, type 2 diabetes. Clinical studies have demonstrated that duodenal mucosal resurfacing of the mucosal layer of the duodenum is a safe procedure that may have a positive impact on glycemic hemostasis in patients with type 2 diabetes.

In some variations, the pulsed or modulated electric field may cause cell lysis in tissue that is at least 50% pore-induced and less than 50% heat-induced. In some variations, a method of treating diabetes may include advancing a pulsed electric field device towards a target tissue of a patient. For example, a patient may be positioned on their left lateral side during the procedure, and the target tissue (e.g., duodenum) may optionally be insufflated (e.g., using CO₂ or saline). The pulsed electric field device may comprise an elongate body and an expandable member comprising an electrode array. Once in the target tissue (e.g., duodenum), the expandable member may be transitioned into an expanded configuration. In some variations, one or more turns of the expandable member may be unrolled to contact the target tissue. In some variations, a visualization device (e.g., endoscope) may be advanced into the target tissue (e.g., duodenum) to visualize, inspect, and/or confirm a treatment area during a procedure. For example, one or more transparent portions of a pulsed electric field device may allow the visualization device to identify a location of the pulsed electric field device within patient anatomy (e.g., an ampulla of the duodenum, bulb of the duodenum). Once the device is located at a desired position within the target tissue, a pulse waveform may be delivered to the electrodes to generate a pulsed electric field to treat a portion of the target tissue. It should be appreciated that any of systems and devices described herein may be used in the methods described here.

In some variations, a method of treating diabetes may include one or more of application of a radially outward force to stretch (e.g., dilate) tissue and application of negative pressure (e.g., suction) to the tissue to facilitate a consistent (e.g., uniform) tissue-electrode interface. For example, tissue stretched or dilated by an expandable member of a pulsed electric field device in the expanded configuration, whether through the application of an outwardly directed radial compression force and/or negative pressure, may have a more uniform tissue thickness, which may aid in a consistent energy delivery and treatment. In some variations, tissue may be in contact with the expandable member in the expanded configuration within the target tissue. A visualization device (e.g., endoscope) may be positioned proximal to the expandable member in the expanded configuration. Then, a suction catheter may be advanced from a lumen of the visualization device into a lumen of the expandable member. The suction catheter may be configured to generate a negative pressure sufficient to pull tissue into and/or through one or more openings (e.g., fluid openings) of the expandable member. This may reduce tissue tenting and/or air pockets over the electrodes and ensure a consistent tissue-electrode interface tissue around an inner circumference of the target tissue. Furthermore, suction may enable a reduction in the radial compression force that may be applied by the expandable member.

In some variations, a radial compression force produced by the expandable member when in an expanded configuration against the target tissue may not be uniform along the length or width of the expandable member, and the electrodes connected or attached to, or integrated with, the expandable member. Suction may be used to supplement some regions of tissue contact that may not be sufficient, or optimal, in terms of connection between the target tissue and the electrodes. In some variations, the negative pressure (e.g., suction) applied to the tissue may be between about 50 mmHg and about 75 mmHg. In some variations, the negative pressure (e.g., suction) applied to the tissue may be applied intermittently or in relatively short time periods at a pressure of between about 100 mmHg and about 250 mmHg. For example, higher negative pressure may be applied in spurts or feathered so as to ensure contact between the tissue and the electrodes without tissue pressure necrosis.

Stretched tissue dilated by the expandable member in the expanded configuration may reduce a wall thickness of the tissue, thereby allowing for a lower dose of energy to treat a predetermined depth of tissue. Stretched tissue may include realigning (e.g., reorienting) cellular structures that increase tissue circumference. Reducing total energy delivery may correspond to a lower overall temperature increase of the tissue, which may increases the safety profile of the treatment procedure as well as promote a faster and safer healing cascade.

In some variations, negative pressure may be applied to the tissue to ensure even contact between tissue and an electrode array during treatment. For example, negative pressure or suction may be applied by an expandable member to a tissue lumen (e.g., duodenum, duodenal tissue) to facilitate tissue apposition with an electrode array of the expandable member. Higher tissue apposition may further enable a reduction in total energy delivery and improved treatment outcomes.

In some variations, stretching the tissue by applying a radially outward force using the expandable member and/or application of negative pressure to the tissue from the expandable member may reduce a range of tissue thicknesses. For example, the expandable member may stretch tissue such that a ratio of manipulated (e.g., compressed/stretched/dilated) tissue thickness to unmanipulated tissue thickness is about 0.5. In some variations, the combination of tissue stretching and application of a pulsed electric field as described herein may synergistically treat a tissue of a patient.

In some variations, a pulsed electric field device as described herein may transition to an expanded configuration to dilate (e.g., stretch, extend) the tissue during a treatment procedure. In some variations, tissue may be treated within a predetermined range of dilation ratios. In some variations, a ratio of dilated to undilated mucosa tissue may be between about 0.40 and about 0.60, between about 0.45 and about 0.55, and about 0.50, including all ranges and sub-values in-between. In some variations, a ratio of dilated to undilated submucosa tissue may be between about 0.15 and about 0.35, between about 0.20 and about 0.30, and about 0.26, including all ranges and sub-values in-between. In some variations, a ratio of dilated duodenum diameter to undilated duodenum diameter may be between about 1.5 and about 2.3, between about 1.7 and about 2.1, and about 1.91, including all ranges and sub-values in-between. In some variations, a ratio of a dilated duodenum diameter to an undilated duodenum diameter may be between about 1.5 and about 2.3, between about 1.7 and about 2.1, and about 1.91, including all ranges and sub-values in-between.

In some variations, a pulsed electric field device may be configured to simultaneously dilate and suction tissue to the electrodes of the expandable member. In some variations, a ratio of suction and dilated to undilated mucosa tissue may be between about 0.40 and about 0.60, between about 0.45 and about 0.55, and about 0.47, including all ranges and sub-values in-between. In some variations, a ratio of suction and dilated to undilated submucosa tissue may be between about 0.20 and about 0.50, between about 0.30 and about 0.40, and about 0.33, including all ranges and sub-values in-between.

In some variations, the suction may be generated by the device itself while in the expanded configuration. Additionally or alternatively, the suction may be generated by a visualization device such as an endoscope. An amount of suction may be configured to secure uniform apposition of tissue to the surface of the expandable member (e.g., electrode surfaces). However, the amount of suction should not exceed a predetermined threshold corresponding to pressure necrosis. In some variations, the negative pressure (e.g., suction) applied to the tissue may be between about 50 mmHg and about 75 mmHg for less than about one minute. In some variations, the negative pressure (e.g., suction) applied to the tissue may be between about 10 mmHg and about 200 mmHg. The amount of suction may be a function of one or more of total surface area of the expandable member, number and size of the openings, time that suction is applied, edge condition of the openings, compliance of tissue, vascularization of tissue, and friability of tissue.

In some variations, an amount of tissue compliance may correspond to an amount of dilation and suction needed to ensure uniform surface contact of the electrodes and the desired tissue treatment. In some variations, the tissue may respond better to less dilation and more suction (or vice versa) depending on compliance and structure. In some variations, apposition may be assessed visually and/or through impedance measurement. In some variations, apposition may be measured using one or more temperature sensors and/or pressure sensors.

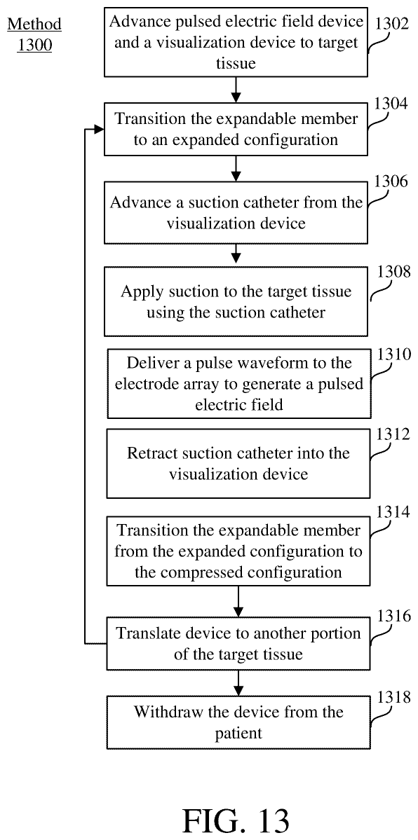

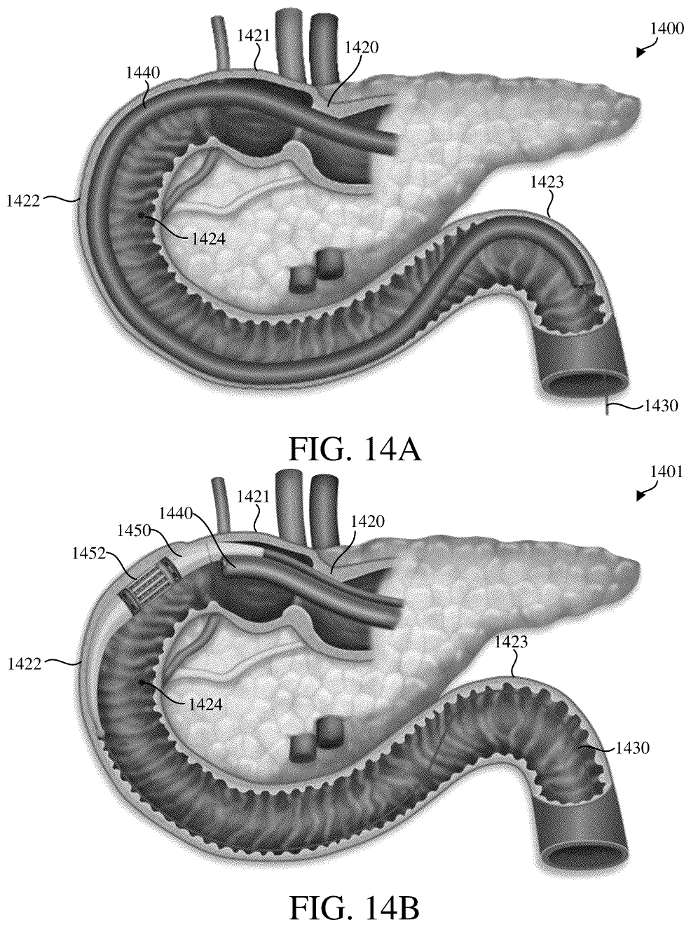

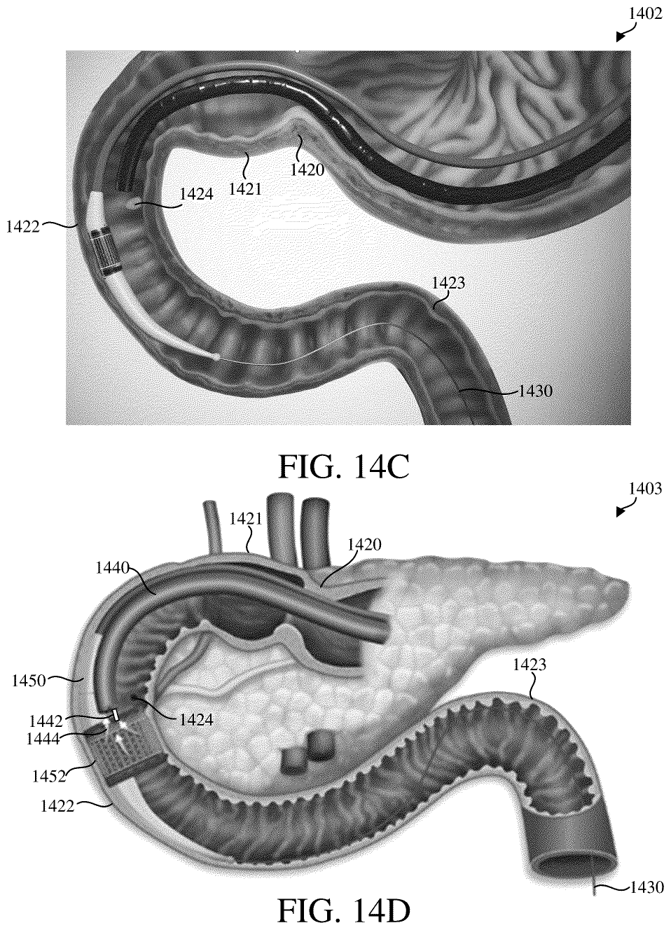

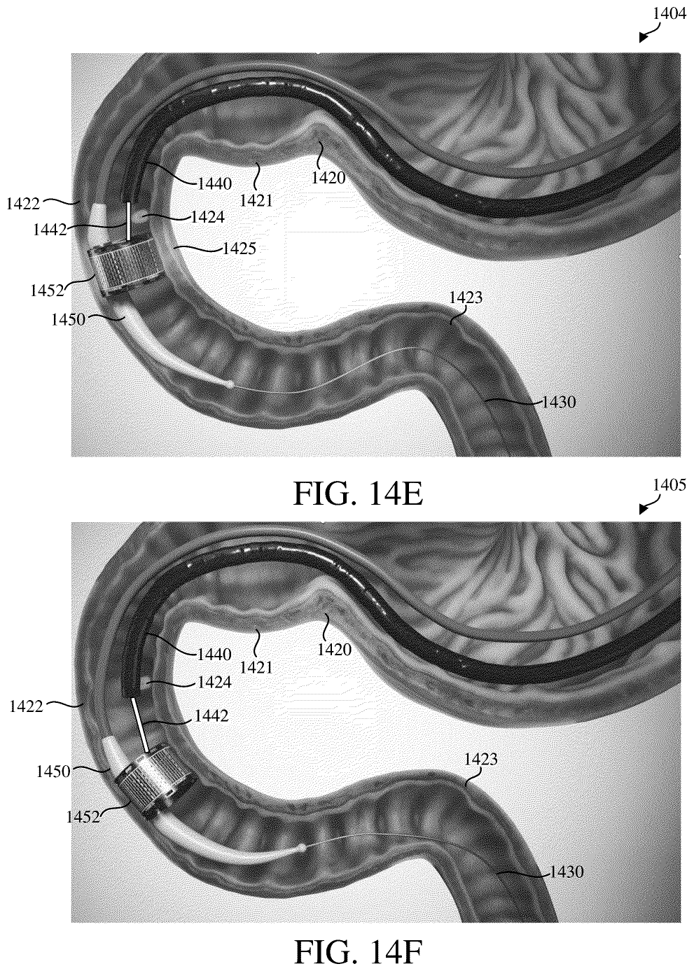

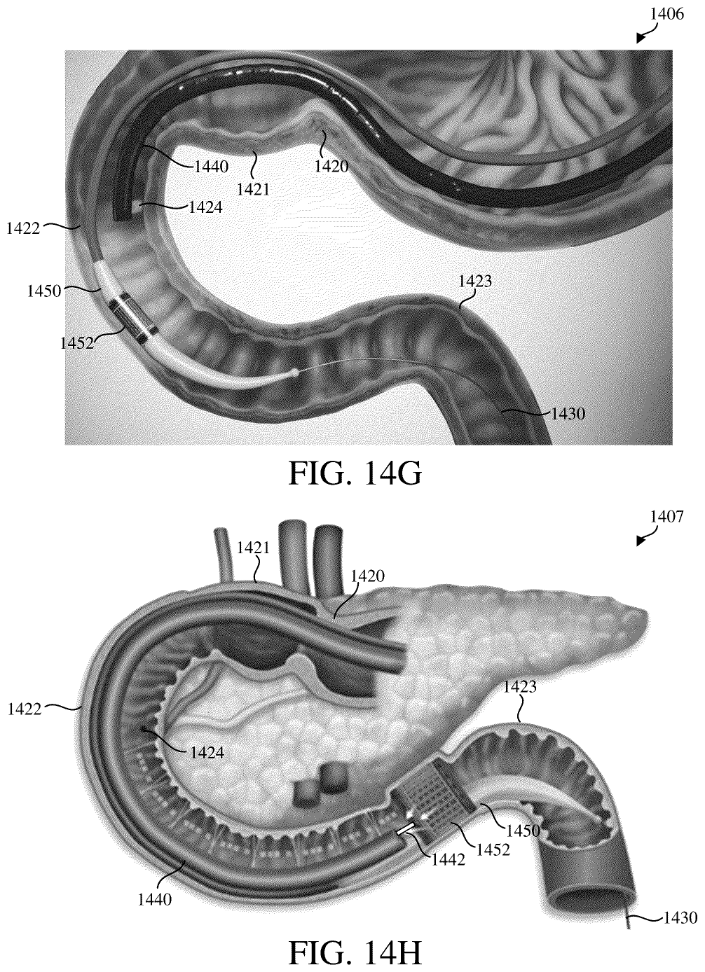

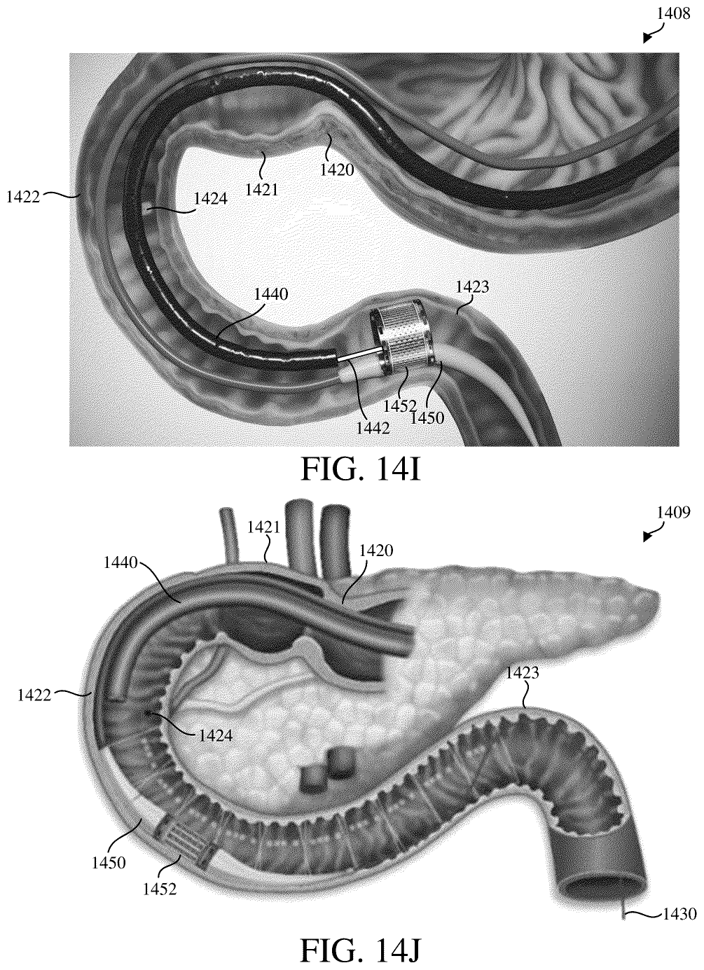

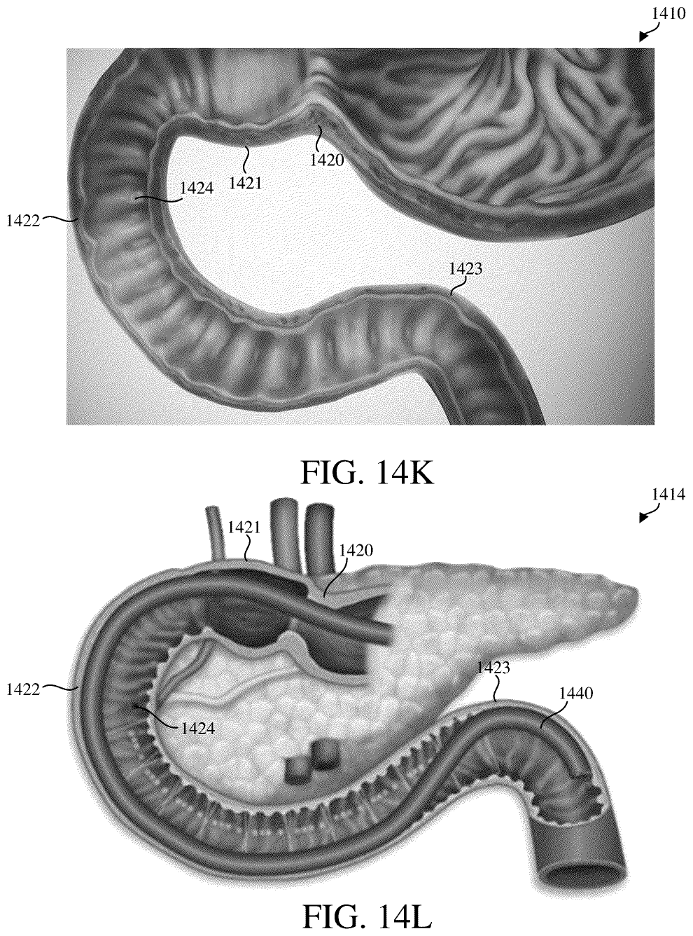

FIG. 13 is a flowchart that generally describes a variation of a method of treating a chronic condition (1300). In some of these variations, a patient may be positioned on their left lateral side or in a prone position during the procedure, and the target tissue may optionally be insufflated (e.g., using CO₂ or saline). The target tissue may include one or more of an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. For ease of explanation, the target tissue discussed with respect to FIG. 13 and illustrated in FIGS. 14A-14L corresponds to a duodenum. In some variations, the method (1300) may be performed under fluoroscopic guidance.

The method (1300) may include advancing a pulsed electric field device and a visualization device to a target tissue of a patient (1302). For example, as shown in the schematic diagram (1401) of FIG. 14A, a visualization device (1440) may be advanced over a guidewire (1430) into a target tissue (e.g., duodenum). For example, the guidewire (1430) and visualization device (1440) (e.g., endoscope) may be advanced through the pylorus (1420), bulb (1421) of the duodenum, descending part (1422), ampulla of Vater (1424), duodenojejunal flexure (1423), and up to and/or beyond the Ligament of Treitz. Then, while maintaining the position of the guidewire (1430), the visualization device (1440) may be withdrawn into the bulb (1421) of the duodenum, as shown in FIG. 14B. The pulsed electric field device (1450) may be advanced into the bulb (1421) and then into the descending part (1422).

In some variations, the pulsed electric field device (1450) and the visualization device (1440) may correspond to any of the pulsed electric field devices (e.g., device (500)) and visualization devices described herein. For example, the pulsed electric field device (1450) may comprise an elongate body, a tissue barrier, and an expandable member (1452) coupled to the elongate body. The expandable member (1452) may comprise an electrode array and one or more fluid openings as described herein. The elongate body may comprise a proximal portion and a distal portion. The tissue barrier may comprise a first portion and a second portion. The first portion may be coupled to a proximal edge of the expandable member and to the proximal portion, and the second portion may be coupled to a distal edge of the expandable member and to the distal portion.

In some variations, throughout the method (1300), the visualization device (1440) may be positioned proximally of the expandable member (1452) while translating (e.g., advancing, withdrawing) the pulsed electric field device (1450). For example, the visualization device (1440) may be positioned such that the expandable member (1452) may be viewed in its entirety, including the proximal end and distal end of the expandable member (1452). The visualization device (1440) may be manipulated independently of the pulsed electric field device (1650).

Accordingly, the pulsed electric field device (1450) (e.g., the expandable member (1452)) may be visualized with the visualization device (1440) while the visualization device (1440) is positioned proximally of the expandable member (1452). FIG. 15A is an image (1500) of an expandable member (1552) of a pulsed electric field device disposed within a bulb (1521) of the duodenum as imaged from the perspective of a distal end of a visualization device (e.g., endoscope). The expandable member (1552) may be in an unexpanded configuration as it is advanced through a bulb of the duodenum (1521).

As shown in the schematic diagram (1401) of FIG. 14B, the pulsed electric field device (1450) may be advanced together with the visualization device (1440) into the descending part (1422) of the duodenum. FIG. 15B is an image (1501) of an expandable member (1552) of a pulsed electric field device disposed within a descending part (1522) of the duodenum from the perspective of a distal end of a visualization device disposed positioned proximally of the expandable member (1552). In some variations, a treatment location within the target tissue may be one or more of proximal and distal to the ampulla of Vater (1424). For example, the expandable member (1452) may be advanced about 1 cm to about 4 cm distal to the ampulla of Vater (1424), as shown in FIG. 14C. Additionally or alternatively, the target tissue may be tissue corresponding to a bulb of the duodenum and/or the ampulla of Vater.

FIG. 15C is an image (1502) of the expandable member (1552) of a pulsed electric field device in the unexpanded configuration disposed within a descending part (1522) of the duodenum. FIG. 15D is an image (1503) of the expandable member (1552) in a partially-expanded configuration.

In step 1304, the expandable member (1452) of the pulsed electric field device (1450) may transition from an unexpanded configuration to an expanded configuration to, for example, engage tissue. As shown in the schematic diagram (1403) of FIG. 14D, the expandable member (1452) in the expanded configuration may dilate a portion of the target tissue in contact with the expandable member (1452), which may be visualized by the visualization device (1440).

The expandable member (1452) in the expanded configuration defines an expandable member lumen. The visualization device (1440) may be positioned proximally of the expandable member (1452) while transitioning the expandable member (1452). In some variations, transitioning the expandable member (1452) may include unrolling and rolling one or more turns of the expandable member. For example, the expandable member (1452) may unroll by one or more turns to transition the expandable member (1452) to an expanded configuration (e.g., unrolled configuration), causing the expandable member to contact the duodenum (1422). In some variations, a second elongate body of the pulsed electric field device (1450) may be rotated relative to the first elongate body to unroll the expandable member (1452). Complete circumferential contact between the expandable member (1452) and duodenum (1422) may improve energy delivery and treatment outcomes. FIG. 15E is an image (1504) of the expandable member (1552) in an expanded configuration.

Although not shown in FIG. 14C for the sake of clarity, transitioning the expandable member into an expanded configuration may concurrently transition the tissue barrier between a rolled configuration (e.g., unexpanded configuration) and an unrolled configuration (e.g., expanded configuration). For example, expanding the tissue barrier may include unrolling one or more turns of the tissue barrier about the first elongate body. The tissue barrier in the unrolled configuration may comprise a pair of right triangles as described in more detail herein with respect to FIGS. 5A-5R and 6A-6G.

Additionally or alternatively, the pulsed electric field device (1450) may comprise a second expandable member (e.g., inflatable member, balloon) (not shown) disposed distal to the expandable member (1452). In some of these variations, the second expandable member may be inflated to aid in one or more of advancement, positioning, and visualization of the pulsed electric field device (1450) relative to the target tissue. For example, one or more portions of the second expandable member may be transparent to allow a visualization device to see through the second expandable member.

In step 1306, a suction catheter (1442) may be advanced from a lumen of the visualization device (1440). For example, the suction catheter (1442) may be advanced from the lumen of the visualization device (1440) into the expandable member lumen. In some variations, the visualization device (1440) may be positioned proximally of the expandable member (1450) as the suction catheter (1442) is advanced from the visualization device (1440). In some variations, the suction catheter (1442) may comprise a suction lumen and correspond to the suction catheter (1200) described with respect to FIGS. 12A and 12B. For example, a distal end of the suction catheter may comprise a plurality of apertures spaced around a circumference of the suction catheter. In some variations, the plurality of apertures may comprise opposing pairs of apertures (e.g., apertures positioned on opposing sidewalls of the suction catheter). In some variations, each aperture of the plurality of apertures may comprises a diameter of up to an inner diameter of the suction catheter. FIG. 15F is an image (1505) of a suction catheter (1542) advanced into a lumen of the expandable member (1552) in the expanded configuration. Alternatively, advancing the suction catheter (1442) may include aligning a distal end of the visualization device (1440) longitudinally with a proximal end of the expandable member. In some variations, a distal end of the suction catheter (1442) may be disposed either within a lumen of the expandable member (1452), proximal to a proximal end of the expandable member (1452), or distal to a distal end of the expandable member (1452). The visualization device (1440) may be disposed distal to the suction catheter (1442) in any of these positions.

In step 1308, suction may be applied to the portion of the target tissue through one or more fluid openings of the expandable member (1450) using the suction catheter (1440). As shown in FIG. 14D, the suction device (1442) may be configured to generate a negative pressure (e.g., suction) (1444) within a lumen of the expandable member (1452) that suctions tissue (1422) to a surface of the expandable member (1452). In some variations, the suction may be applied radially by the suction catheter. Additionally or alternatively, the device (1450) may be configured to generate negative pressure to suction tissue (1442) to the surface of the expandable member (1452). The close contact between the tissue (1442) and the expandable member (1452) may improve energy delivery and treatment outcomes. One or more pulse waveforms may be delivered while suction is being applied. In some variations, suction may be applied during delivery of a pulse waveform and reduced during time periods when pulsed electric field energy is not delivered. For example, suction may be reduced (e.g., halted) during a time period after energy delivery, and when one or more of the device (1450) and visualization device (1440) are advanced within the duodenum. Thus, suction may be generated intermittently throughout a treatment procedure (e.g., concurrent with energy delivery). An amount of suction applied to one or more portions of tissue may be as described herein.

In step 1310, one or more pulse waveforms may be delivered to an electrode array of an expandable member to generate a pulsed or modulated electric field. In some variations, the electrode array may have a plurality of sections arranged circumferentially about the first elongate body. For example, the electrode array may have two, three, four, or five sections exposed to tissue when in the expanded configuration. The operator may confirm tissue contact with a predetermined number of electrode sections and may select the corresponding electrode sections for energy delivery from a signal generator.

In some variations, the signal generator may be configured to deliver a pulsed electric field waveform to two or more non-proximate sections of the plurality of sections in a predetermined sequence. In some variations, the visualization device may allow vision of the fiducial markers, e.g., numbers or other indicators, to aid in determining how many of the electrode sections have been unrolled or expanded to contact the target tissue. In some variations, the predetermined sequence may comprise all of the electrode sections. In some variations, the predetermined sequence may comprise a subset of available electrode sections. In some variations, the predetermined sequence may comprise only those electrode sections confirmed to be unrolled or expanded and in apposition against the target tissue.

For example, the signal generator may generate a waveform sequence (e.g., interleaving waveform) having an inter-section delay between sections of an electrode array. In particular, the predetermined sequence may comprise an inter-section delay between delivery of a first pulsed electric field waveform to a first section of the plurality of sections and a second pulsed electric field waveform to a second section of the plurality of sections. The first and second pulsed electric field waveforms may be the same or different. In some variations, the inter-section delay may be between about 10 ms and about 4000 ms. In some variations, the first and second sections are non-adjacent (e.g., not immediately next to each other) sections. In some variations, the predetermined sequence may further comprise an intra-section delay between delivery of the first pulsed electric field waveform to the first section and delivery of a second pulsed electric field waveform to the first section.

In some variations, the intra-section delay may be between about 1 seconds and about 10 seconds. In some variations, the first and second pulsed electric field waveforms may comprise a series of between about 10 bipolar pulses and about 500 bipolar pulses. In some variations, each of the bipolar pulses may comprise a pulse width between about 1 μs and about 3 μs.

In some variations, the first and second pulsed electric field waveforms may comprise the same number of bipolar pulses. In some variations, the first and second pulsed electric field waveforms may comprise a different number of bipolar pulses. In some variations, between about 0.05 J per bipolar pulse and about 0.5 J per bipolar pulse may be delivered to the electrode array. In some variations, an instantaneous power between about 26,000 W per bipolar pulse and about 70,000 W per bipolar pulse may be delivered by the electrode array. In some variations, the predetermined sequence may be repeated between about 5 and about 15 times. In some variations, activation of the plurality of sections may have a cumulative activation time between about 0.1 ms and about 10 ms over a treatment time between about 30 seconds and about 35 seconds. In some variations, the predetermined sequence may comprise a duty cycle between about 0.003% and about 0.004%. In some variations, the plurality of sections may comprise between abut one section and about ten sections, between about two sections and eight sections, between about three sections and seven sections, and up to five sections, including all ranges and sub-values in-between. In some variations, the electrode array may comprise a surface area between about 4 square centimeters and about 42 square centimeters. In some variations, each section of the plurality of sections may comprise a plurality of electrodes. In some variations, each section of the plurality of sections may comprise between 10 and 18 electrodes.

In some variations, a pulsed electric field waveform (e.g., interleaving waveform) may be delivered in a predetermined sequence to each of a first section and a second section non-proximate to the first section. The predetermined sequence may have an inter-section delay between the first and second sections of an electrode array.

In some variations, the intra-section delay may be between about 10 ms and about 10,000 ms, between about 5000 ms and about 10,000 ms, between about 10 ms and about 5000 ms, and between about 2000 ms and about 8000 ms, including all ranges and sub-values in-between. In some variations, the first section may be re-activated after an intra-section delay relative to a previous activation of the first section. For example, the intra-section delay may be between about 3 seconds and about 5 seconds. In some variations, the pulsed electric field waveform may comprise a series of between about 40 bipolar pulses and about 60 bipolar pulses. In some variations, each of the bipolar pulses may comprise a pulse width between about 1 μs and about 3 μs. In some variations, activating each of the first and second sections may deliver between about 0.05 J per bipolar pulse and about 0.5 J per bipolar pulse. In some variations, activating each of the first and second sections may deliver an instantaneous power between about 38,800 W per bipolar pulse and about 41,250 W per bipolar pulse. In some variations, each of the bipolar pulses may comprise a positively-charged portion and a negatively-charged portion each having a pulse width between about 1.3 μs and about 1.5 μs. In some variations, each of the bipolar pulses may comprise a time interval between the positively-charged and negatively-charged portions. In some variations, the time interval may be between about 0.05 μs and about 0.1 μs.

In some variations, the first and second sections are non-proximate. In some variations, the electrode array may further comprise one or more of a third section, a fourth section, and a fifth section. In some variations, the predetermined sequence may further comprise activating the one or more of the third section, fourth section, and fifth section with the inter-section delay between activation of successive sections. In some variations, the first and second sections may be activated for a cumulative activation time between about 0.1 ms and about 10 ms over a treatment time between about 30 and about 35 seconds. In some variations, the first wherein the predetermined sequence comprises a duty cycle between about 0.003% and about 0.004%. In some variations, the predetermined sequence may be repeated between about 5 and about 15 times.

The characteristics associated with the pulse waveform may correspond to an amount of energy generated by the electrode array, which in turn may be applied to tissue. The amount of energy may correspond to one or more electric fields generated by the electrode array.

In some variations, the same portion of tissue may be treated multiple times (e.g., double treated). Treating a same portion of tissue a plurality of times (e.g., two times, three times, four times) may increase the percentage of the tissue in the portion having been treated, thus yielding a more complete lesion leading to improved outcomes. The same pulse waveform energy parameters as first delivered in step 1310 or different pulse waveform energy parameters may be delivered to the same portion of tissue (e.g., gastrointestinal tract, including but not limited to, the duodenum, pylorus, esophagus, stomach, small intestine, and large intestine) when treating the same portion of tissue a plurality of times. In some variations, the pulsed waveform comprises a first pulsed waveform, and delivering at least a second pulsed waveform to the electrode array to generate a second pulsed or modulated electric field thereby treating at least a portion of the tissue previously treated. A plurality of treatments at the same portion of tissue improves the homogeneity of the treatment rather than a depth of penetration.

FIG. 14E depicts treated tissue (1425) corresponding to the tissue in contact with the expandable member (1452) having received the pulse waveform that generates a pulsed electric field. In some variations, the suction catheter (1442) may be configured to apply suction or negative pressure to tissue during energy delivery to increase the apposition of the treated tissue (1425) to an electrode array of the expandable member (1452). In some variations, the visualization device (1440) may be positioned proximally of the expandable member (1452) while delivering the pulsed waveform.

In some variations, the method may include measuring a temperature of the tissue during treatment using a temperature sensor as described herein, and the measured temperature may be between about 37° C. and about 45° C. (e.g., an increase of between about 3° C. and 8° C.) during delivery of the pulsed waveform. Put another way, delivery of the pulsed or modulated electric field created by the pulsed waveforms described herein may produce an increase in tissue temperature of between about 3° C. and 8° C. and a resultant tissue temperature of between about 37° C. and about 45° C. For example, a target temperature achieved by application of the pulsed or modulated electric fields created by the pulsed waveforms described herein may be at about 41° C., which may correspond to about a 4° C. to about 5° C. temperature increase in the tissue. In some variations, the method may include increasing a temperature of the tissue to about 41° C. before delivering the pulsed waveform.

In some variations, as described in more detail herein, tissue may be compressed during treatment with the pulsed or modulated electric field. In these variations, the pulsed or modulated electric field may be a therapeutic electric field that treats tissue at a compressed tissue depth of between about 0.25 mm and about 0.75 mm and at an uncompressed tissue depth of between about 0.50 mm and about 1.5 mm.

In step 1312, once energy delivery is completed to the portion (1424) of tissue, the suction catheter (1442) may be withdrawn (e.g., retracted) from the lumen of the expandable member (1452) and optionally withdrawn into the lumen of the visualization device (1440). For example, FIG. 15G is an image (1506) of the expandable member (1552) in the expanded configuration after energy delivery and retraction of the suction catheter into the visualization device.

In step 1314, the expandable member (1452) may be transitioned from the expanded configuration to the compressed configuration (or the partially or semi-expanded configuration). For example, FIG. 15H is an image (1507) of the expandable member (1552) in the unexpanded configuration after energy delivery such that the expandable member (1552) disengages from the treated tissue (1520) of the duodenum (5630). This allows the pulsed electric field device and the visualization device to be slidably translated together relative to the duodenum. In some variations, the suction catheter (1542) may be used to remove residual fluids and/or improve visualization. The treated tissue (1520) may be inspected for signs of thermal or physical injury. For example, FIG. 15I is an image (1508) of the expandable member (1552) in an unexpanded configuration after treating tissue (1520). For example, treated tissue (1520) may be identified via the suction marks (1523).

In step 1316, the pulsed electric field device may be translated (e.g., advanced) to another portion of tissue to be treated where steps 1304-1316 may be repeated as desired. For example, the pulsed electric field device and/or visualization device may be advanced through the duodenum multiple times to repeat the energy delivery process described herein. In some variations, the proximal edge of the expandable member (1552) (e.g., electrode array) may be aligned against an edge of the treated tissue (1520).

In some variations, the duodenum may be treated over about 2 portions to about 20 portions, about 6 portions to about 15 portions, about 6 portions to about 10 portions, about 10 portions to about 12 portions, including all ranges and sub-values in-between. In some variations, a total treatment length of tissue may be between about 6 cm and about 20 cm. In some variations, a portion of the tissue may have a circumference between about 22 mm and an average of about 25 mm. In some variations, more than about 60 percent of a circumference of a portion of the duodenum may be treated.

In step 1318, the pulsed electric field device and the visualization device may be withdrawn from the patient. The pulsed electric field device and the visualization device may be withdrawn from the patient sequentially or simultaneously. For example, the pulsed electric field device and visualization device may be withdrawn from the patient, and the visualization may be reintroduced into the patient to inspect the treated tissue.

### EXAMPLES

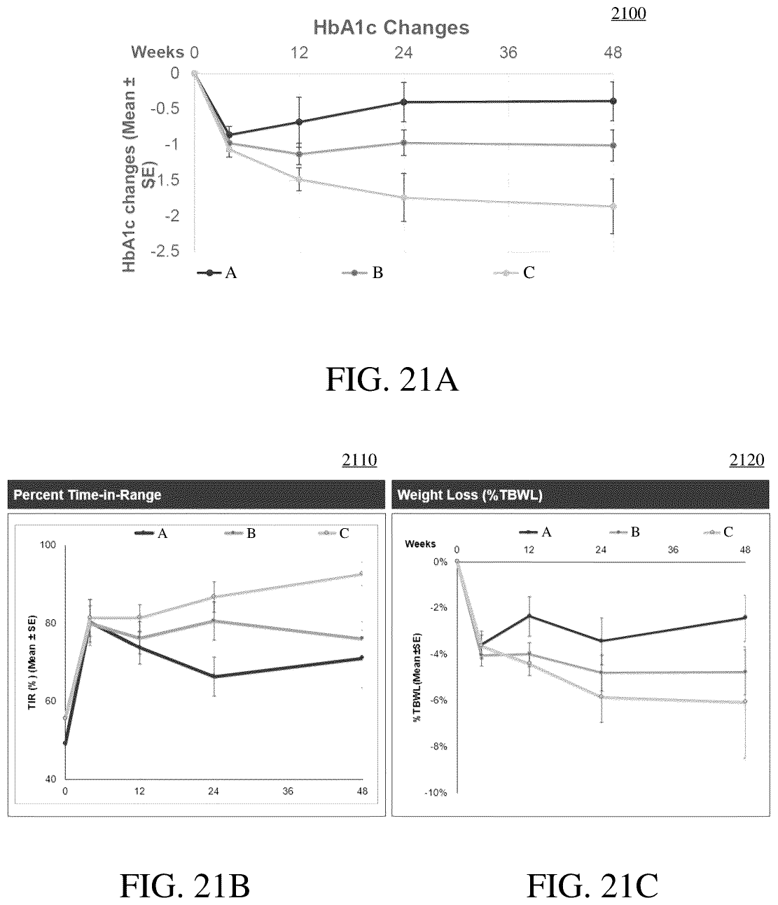

As described herein, the systems and devices described herein deliver energy to tissue to treat a chronic condition. For example, exemplary devices and energy delivery parameters (e.g., protocols A, B, C) improve one or more of HbA1c, time-in-range (TIR), and weight loss (e.g., % TBWL) for insulin naïve Type 2 diabetes patients, as shown in FIG. 21A-21C. In particular, protocols A and B use a first pulsed electric field device including an expandable member having a maximum diameter of about 32 mm, and protocol C uses a second pulsed electric field device including an expandable member having a maximum diameter of about 45 mm. Protocol A delivers a single dose at each treatment site of a pulse waveform having a voltage of about 600 volts at about 350 kHz. Protocols B and C deliver a double dose at each treatment site of a pulse waveform having a voltage of about 750 volts at about 350 kHz.

FIG. 21A is a plot (2100) of dose-based changes in HbA1c over time (e.g., weeks) in response to a duodenal mucosal resurfacing procedure for insulin naïve Type 2 diabetes patients using protocols A, B, and C. FIG. 21B is a plot (2110) of time-in-range-based changes over time (e.g., weeks) in response to a duodenal mucosal resurfacing procedure for insulin naïve Type 2 diabetes patients using protocols A, B, and C. FIG. 21C (2120) is a plot of weight-based changes over time (e.g., weeks) in response to a duodenal mucosal resurfacing procedure for insulin naïve Type 2 diabetes patients using protocols A, B, and C. As shown in FIGS. 21A-21C, the improvement in HbA1c, TIR, and weight loss are at least maintained through 48 weeks. For example, FIG. 21A shows improvements in HbA1c in patients undergoing Protocol C continue to improve from week 0 through week 48. Similar improvement is shown in FIG. 21C with respect to weight loss for Protocol C patients.

### Exemplary Embodiments

In addition to the claims set out below, the following exemplary embodiments are, without limitation, directed to aspects of the present disclosure.

Embodiment 1. A method of treating a target tissue, comprising:
advancing a pulsed electric field device and a visualization device to the target tissue of a patient, the pulsed electric field device comprising an elongate body and an expandable member coupled to the elongate body, wherein the expandable member comprises an electrode array and one or more fluid openings;
transitioning the expandable member into an expanded configuration;
advancing a suction catheter from a lumen of the visualization device;
applying suction to the portion of the target tissue through the one or more fluid openings of the expandable member using the suction catheter; and
delivering a pulsed waveform to the electrode array to generate a pulsed or modulated electric field thereby treating the target tissue.

Embodiment 2. The method of embodiment 1, wherein the visualization device is positioned proximally of the expandable member while a) advancing the pulsed electric field device, b) transitioning the expandable member, c) applying suction, and d) delivering the pulsed waveform.

Embodiment 3. The method of embodiment 1 further comprising visualizing the expandable member with the visualization device while the visualization device is positioned proximally of the expandable member.

Embodiment 4. The method of embodiment 1, wherein the expandable member in the expanded configuration defines an expandable member lumen, wherein the suction catheter is advanced from the lumen of the visualization device into the expandable member lumen.

Embodiment 5. The method of embodiment 1, wherein the suction catheter comprises a wire.

Embodiment 6. The method of embodiment 5, wherein a proximal end of the wire comprises a loop.

Embodiment 7. The method of embodiment 6, wherein the suction catheter comprises a suction lumen and a diameter of the wire is less than a diameter of the suction lumen.

Embodiment 8. The method of embodiment 6, wherein a diameter of the loop is larger than a diameter of the lumen of the visualization device.

Embodiment 9. The method of embodiment 7, wherein a proximal end of the suction lumen couples to a distal end of the wire.

Embodiment 10. The method of embodiment 1, wherein advancing the suction catheter includes aligning a distal end of the visualization device longitudinally with a proximal end of the expandable member.

Embodiment 11. The method of embodiment 1, wherein a distal end of the suction catheter comprises a plurality of apertures spaced around a circumference of the suction catheter.

Embodiment 12. The method of embodiment 11, wherein the plurality of apertures comprises opposing pairs of apertures.

Embodiment 13. The method of embodiment 11, wherein each aperture of the plurality of apertures comprises a diameter of up to an inner diameter of the suction catheter.

Embodiment 14. The method of embodiment 1, wherein the pulsed waveform is delivered simultaneously with applying suction to the portion of the target tissue.

Embodiment 15. The method of embodiment 1, wherein transitioning the expandable member into the expanded configuration dilates a portion of the target tissue.

Embodiment 16. The method of embodiment 1 wherein the suctioned tissue is received through the one of more fluid openings when applying the suction.

Embodiment 17. The method of embodiment 1, wherein the target tissue is duodenal tissue proximal and/or distal to an ampulla of Vater.

Embodiment 18. The method of embodiment 1, wherein the target tissue is tissue corresponding to a bulb of the duodenum.

Embodiment 19. The method of embodiment 1, wherein the pulsed electric field device is advanced together with the visualization device into the duodenum.

Embodiment 20. The method of embodiment 1, wherein the suction is applied at least radially and longitudinally by the suction catheter.

Embodiment 21. The method of embodiment 1, wherein treating the target tissue treats one or more of a metabolic disorder and cancer.

Embodiment 22. The method of embodiment 20, wherein the metabolic disorder comprises one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes.

Embodiment 23. The method of embodiment 1, wherein the target tissue comprises one or more of a duodenum, a pylorus, an esophagus, a stomach, a small intestine, and a large intestine.

Embodiment 24. A suction catheter, comprising:
a first elongate body comprising a proximal end, a distal end, and a lumen between the proximal and distal ends, the distal end of the first elongate body comprises a plurality of apertures spaced around a circumference of the first elongate body; and
a second elongate body coupled to a proximal end of the first elongate body, wherein a diameter of the second elongate body is less than a diameter of the first elongate body.

Embodiment 25. The suction catheter of embodiment 24, wherein the second elongate body comprises one or more of a wire, a cable, a coil, and a braid.

Embodiment 26. The suction catheter of embodiment 24, wherein the plurality of apertures comprises opposing pairs of apertures, wherein each aperture of the plurality of apertures comprises a shape including one or more of a circle, ellipse, and polygon.

Embodiment 27. The suction catheter of embodiment 24, wherein a diameter of each aperture of the plurality of apertures is equal to or less than an inner diameter of the suction catheter.

Embodiment 28. The suction catheter of embodiment 24, wherein a proximal end of the second elongate body comprises a loop.

Embodiment 29. The suction catheter of embodiment 24, wherein a diameter of the second elongate body is less than a diameter of the lumen.

Embodiment 30. The suction catheter of embodiment 28, wherein a diameter of the loop is larger than a diameter of the lumen.

Embodiment 31. The suction catheter of embodiment 24, wherein the second elongate body is coupled to an inner surface of the lumen at a proximal end of the first elongate body.

Embodiment 32. The suction catheter of embodiment 24, wherein the suction catheter is configured to apply suction at least radially and longitudinally through the plurality of apertures.

Embodiment 33. A system for treating tissue comprising:
the suction catheter of embodiment 24;
a visualization device comprising a visualization lumen,
wherein the suction catheter is configured to be advanced through the

visualization device lumen.

Embodiment 34. The system of embodiment 33 further comprising a pulsed electric field device comprising an elongate body and an expandable member coupled to the elongate body, wherein the expandable member comprises an electrode array and one or more fluid openings, wherein the suction catheter is configured to apply suction to tissue through the one or more fluid openings of the expandable member.

Embodiment 35. The system of embodiment 34, wherein the expandable member comprises an expandable member lumen in an expanded configuration, and wherein the first elongate body is configured to be received within the expandable member lumen to assist in applying suction through the one or more fluid openings of the expandable member.

Embodiment 36. The suction catheter of embodiment 33, wherein a proximal end of the second elongate body is configured to restrict translation of the suction catheter through the visualization device.

Embodiment 37. A device, comprising:
a first elongate body comprising a proximal portion, a distal portion, and a lumen therethrough;
an expandable member comprising an electrode array, an inner end, and an outer end coupled to the first elongate body, wherein the expandable member is disposed between the proximal portion and the distal portion; and
a tissue barrier comprising a first portion and a second portion, the first portion coupled to a distal edge of the expandable member and to the distal portion of the elongate body, and the second portion coupled to a proximal edge of the expandable member and to the proximal portion of the elongate body,
wherein the tissue barrier is configured to achieve a compressed configuration and an expanded configuration.

Embodiment 38. The device of embodiment 37, wherein the tissue barrier is configured to be rolled about the first elongate body.

Embodiment 39. The device of embodiment 37, further comprising a second elongate body at least partially positioned within the lumen of the first elongate body, wherein the expandable member is rolled about the second elongate body, and the inner end is coupled to the second elongate body.

Embodiment 40. The device of embodiment 37, wherein the proximal portion comprises a proximal dilator and the distal portion comprise a distal dilator.

Embodiment 41. The device of embodiment 37, wherein the tissue barrier is configured to transition between a rolled configuration and an unrolled configuration.

Embodiment 42. The device of embodiment 41, wherein the second elongate body is configured to rotate relative to the first elongate body to transition the tissue barrier between a collapsed rolled configuration and an expanded unrolled configuration.

Embodiment 43. The device of embodiment 42, wherein the first and second portions of the tissue barrier in the unrolled configuration form a pair of right triangles.

Embodiment 44. The device of embodiment 43, wherein each triangle of the pair of right triangles comprises an acute angle of between about 20 degrees and about 90.

Embodiment 45. The device of embodiment 37, wherein a shape of the first portion and the second portion is the same.

Embodiment 46. The device of embodiment 37, wherein the tissue barrier comprises a plurality of turns about the first elongate body.

Embodiment 47. The device of embodiment 37, wherein the tissue barrier is rolled about a longitudinal axis of the first elongate body.

Embodiment 48. The device of embodiment 37, wherein the tissue barrier comprises a durometer between about 10 Shore A and about 100 Shore A.

Embodiment 49. The device of embodiment 37, wherein a distance between the first portion and the second portion is between about 3 mm and about 20 mm.

Embodiment 50. The device of embodiment 37, wherein the tissue barrier comprises a thickness of between about 0.125 mm and about 0.8 mm.

Embodiment 51. The device of embodiment 37, wherein the tissue barrier couples to each of the proximal edge and the distal edge for a length of between about 25 mm and about 130 mm.

Embodiment 52. A method of treating a target tissue, comprising:
advancing a pulsed electric field device to the target tissue of a patient, the pulsed electric field device comprising an elongate body, a tissue barrier, and an expandable member coupled to the elongate body, wherein the expandable member comprises an electrode array and the elongate body comprises a proximal portion and a distal portion;
transitioning the expandable member into an expanded configuration to contact a portion of the target tissue and to expand the tissue barrier; and
delivering a pulsed waveform to the electrode array to generate a pulsed or modulated electric field thereby treating the target tissue.

Embodiment 53. The method of embodiment 52, wherein the tissue barrier comprises a first portion and a second portion, the first portion coupled to a proximal edge of the expandable member and to the proximal portion, and the second portion coupled to a distal edge of the expandable member and to the distal portion.

Embodiment 54. The method of embodiment 52, wherein transitioning the expandable member comprises unrolling one or more turns of the expandable member.

Embodiment 55. The method of embodiment 52, wherein transitioning the expandable member comprises unrolling one or more turns of the tissue barrier about the first elongate body.

Embodiment 56. The method of embodiment 52, wherein transitioning the expandable member comprises rotating the second elongate body relative to the first elongate body.

Embodiment 57. The method of embodiment 52, wherein the tissue barrier is configured to transition between a rolled configuration and an unrolled configuration.

Embodiment 58. The method of embodiment 57, wherein the tissue barrier in the unrolled configuration comprises a pair of right triangles.

Embodiment 59. A device, comprising:
a first elongate body comprising a lumen;
an expandable member comprising an inner end, an outer end coupled to the first elongate body, and an electrode array, wherein the expandable member is disposed between a proximal portion and a distal portion of the first elongate body; and
a cover coupled between the outer end of the expandable member and the first elongate body, the cover configured to overlap a portion of the expandable member.

Embodiment 60. The device of embodiment 59, further comprising a second elongate body at least partially positioned within the lumen, wherein the expandable member is rolled about the second elongate body, and the inner end is coupled to the second elongate body.

Embodiment 61. The device of embodiment 59, wherein the cover overlaps the portion of the expandable member as the expandable member rolls about the first elongate body.

Embodiment 62. The device of embodiment 59, wherein the cover comprises a concave shape having a radius of curvature between about 6 mm and about 16 mm.

Embodiment 63. The device of embodiment 59, wherein the cover comprises a width of between about 23 mm and about 38 mm.

Embodiment 64. The device of embodiment 59, wherein a length of the cover is greater than or equal to a length of the expandable member.

Embodiment 65. The device of embodiment 59, wherein the cover comprises one or more atraumatic edges.

Embodiment 66. The device of embodiment 59, wherein the cover is configured to reduce contact between tissue and the second elongate body.

Embodiment 67. The device of embodiment 59, wherein the cover comprises a slit configured to slidably receive the expandable member therethrough.

Embodiment 68. The device of embodiment 67, wherein the cover comprises a first portion facing an outer side of the expandable member and a second portion facing an inner side of the expandable member.

Embodiment 69. The device of embodiment 68, wherein the second portion is coupled to the first elongate body.

Embodiment 70. The device of embodiment 67, wherein the slit is parallel to a longitudinal axis of the first elongate body.

Embodiment 71. The device of embodiment 70, wherein rolling and unrolling the expandable member translates the expandable member through the slit.

It should be understood that the examples and illustrations in this disclosure serve exemplary purposes and departures and variations such as the number of electrodes and devices, and so on can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within +10% of 100 (e.g., from 90 to 110). The terms “about” and “approximately” may be used interchangeably.

The specific examples and descriptions herein are exemplary in nature and variations may be developed by those skilled in the art based on the material taught herein without departing from the scope of the present invention, which is limited only by the attached claims.

1. A system for treating tissue, comprising:
a pulsed electric field device configured and comprising an elongate body and an expandable member coupled to the elongate body, wherein the expandable member comprises an electrode array having a plurality of sections; and
a signal generator coupled to the electrode array, wherein the signal generator is configured to deliver a pulsed electric field waveform to two or more non-proximate sections of the plurality of sections in a predetermined sequence.

2. The system of claim 1, wherein the predetermined sequence comprises an inter-section delay between delivery of a first pulsed electric field waveform to a first section of the plurality of sections and a second pulsed electric field waveform to a second section of the plurality of sections

3. The system of claim 2, wherein the inter-section delay is between about 10 ms and about 4000 ms.

4. The system of claim 2, wherein the first and second sections are non-proximate sections.

5. The system of claim 2, wherein the intra-section delay is between about 1 seconds and about 10 seconds.

6. The system of claim 2, wherein the first and second pulsed electric field waveforms comprise a series of between about 10 bipolar pulses and about 500 bipolar pulses.

7. The system of claim 6, wherein each of the bipolar pulses comprises a pulse width between about 1 μs and about 10 μs.

8. The system of claim 1, wherein the first and second pulsed electric field waveforms comprises the same number of bipolar pulses.

9. The system of claim 1, wherein the first and second pulsed electric field waveforms comprise a different number of bipolar pulses.

10. The system of claim 1, wherein the electrode array is configured to deliver between about 0.05 J per bipolar pulse and about 0.5 J per bipolar pulse.

11. The system of claim 1, wherein the electrode array is configured to deliver an instantaneous power between about 26,000 W per bipolar pulse and about 70,000 W per bipolar pulse.

12. The system of claim 1, wherein the signal generator is configured to repeat the predetermined sequence between about 5 and about 15 times.

13. The system of claim 1, wherein the signal generator is configured to activate the plurality of sections for a cumulative activation time between about 0.1 ms and about 10 ms over a treatment time between about 30 seconds and about 35 seconds.

14. The system of claim 1, wherein the predetermined sequence comprises a duty cycle between about 0.001% and about 0.05%.

15. The system of claim 1, wherein the plurality of sections comprises up to about ten sections.

16. The system of claim 1, wherein the electrode array comprises a surface area between about 4 square centimeters and about 42 square centimeters.

17. The system of claim 1, wherein each section of the plurality of sections comprises a plurality of electrodes.

18. The system of claim 1, wherein each section of the plurality of sections comprises between about 8 electrodes and about 18 electrodes.

19. A method of treating tissue, comprising:
advancing a pulsed electric field device to a target tissue of a patient, the pulsed electric field device comprising an elongate body and an expandable member coupled to the elongate body, wherein the expandable member comprises an electrode array having at least a first section and a second section coupled to a signal generator, the first section non-proximate to the second section; and
treating tissue by providing a pulsed electric field waveform from the signal generator to each of the first and second sections in a predetermined sequence to activate the first section followed by the second section after an inter-section delay, wherein activation of each of the first and second sections generates a therapeutic electric field.

20. The method of claim 19, wherein the delay is between about 10 ms and about 4000 ms.

21. The method of claim 19 further comprising re-activating the first section after an intra-section delay relative to a previous activation of the first section.

22. The method of claim 21, wherein the intra-section delay is between about 1 seconds and about 10 seconds.

23. The method of claim 19, wherein the pulsed electric field waveform comprises a series of between about 10 bipolar pulses and about 500 bipolar pulses.

24. The method of claim 23, wherein each of the bipolar pulses comprises a pulse width between about 1 μs and about 10 μs.

25. The method of claim 24, wherein activating each of the first and second sections delivers between about 0.05 J per bipolar pulse and about 0.5 J per bipolar pulse.

26. The system of claim 24, wherein activating each of the first and second sections delivers an instantaneous power between about 26,000 W per bipolar pulse and about 70,000 W per bipolar pulse.

27. The method of claim 19, wherein the pulsed electric field waveform delivered to the first section comprises a different number of bipolar pulses than the pulsed electric field waveform delivered to the second section.

28. The method of claim 19, wherein the pulsed electric field waveform delivered to the first section comprises an equivalent number of bipolar pulses as the pulsed electric field waveform delivered to the second section.

29. The method of claim 19, wherein the first and second sections are non-proximate.

30. The method of claim 19, wherein the electrode array further comprises one or more of a third section, a fourth section, and a fifth section.

31. The method of claim 30, wherein the predetermined sequence further comprises activating the one or more of the third section, fourth section, and fifth section with the inter-section delay between activation of successive sections.

32. The method of claim 19, wherein the first and second sections are activated for a cumulative activation time between about 0.1 ms and about 10 ms over a treatment time between about 30 and about 35 seconds.

33. The method of claim 19, wherein the first wherein the predetermined sequence comprises a duty cycle between about 0.001% and about 0.05%.

34. The method of claim 19, further comprising repeating the predetermined sequence between about 5 and about 15 times.

35. A system for treating tissue, comprising:
a pulsed electric field device comprising an elongate body and an expandable member coupled to the elongate body, wherein the expandable member comprises an electrode array having a plurality of sections; and
a signal generator coupled to the electrode array, wherein the signal generator is configured to deliver a series of bipolar pulses to two or more non-proximate sections of the plurality of sections in a predetermined sequence for a cumulative activation time of between about 1 ms and about 2 ms over a treatment period between about 30 seconds and about 35 seconds, wherein each bipolar pulse comprises a pulse width between about 2.5 μs and about 3 μs, and wherein the electrode array is configured to deliver between about 0.1 J per bipolar pulse and about 0.2 J per bipolar pulse and an instantaneous power between about 38,800 W per bipolar pulse and about 41,250 W per bipolar pulse.

35. The system of claim 35, wherein each of the bipolar pulses comprises a positively-charged portion and a negatively-charged portion each having a pulse width between about 1.3 μs and about 1.5 μs.

36. The system of claim 35, wherein each of the bipolar pulses comprises a time interval between the positively-charged and negatively-charged portions.

37. The system of claim 36, wherein the time interval is between about 0.05 μs and about 0.1 μs.

38. The system of claim 35, wherein the electrode array comprises a surface area between about 4 square centimeters and about 5 square centimeters.
